SEQUENCE LISTING (171 sequences)

SEQ ID NO: 1 - protein, 4 aa, synthetic construct
HHHH

SEQ ID NO: 2 - protein, 4 aa, synthetic construct
KHKK

SEQ ID NO: 3 - protein, 4 aa, synthetic construct
KKHK

SEQ ID NO: 4 - protein, 4 aa, synthetic construct
KKKH

SEQ ID NO: 5 - protein, 4 aa, synthetic construct
KHKH

SEQ ID NO: 6 - protein, 4 aa, synthetic construct
HKHK

SEQ ID NO: 7 - protein, 4 aa, synthetic construct
KKKK

SEQ ID NO: 8 - protein, 4 aa, synthetic construct
KKRK

SEQ ID NO: 9 - protein, 4 aa, synthetic construct
KRKK

SEQ ID NO: 10 - protein, 4 aa, synthetic construct
KRRK

SEQ ID NO: 11 - protein, 4 aa, synthetic construct
RKKR

SEQ ID NO: 12 - protein, 4 aa, synthetic construct
RRRR

SEQ ID NO: 13 - protein, 4 aa, synthetic construct
KGKK

SEQ ID NO: 14 - protein, 4 aa, synthetic construct
KKGK

SEQ ID NO: 15 - protein, 5 aa, synthetic construct
HBHBH

SEQ ID NO: 16 - protein, 5 aa, synthetic construct
RRRRR

SEQ ID NO: 17 - protein, 5 aa, synthetic construct
KKKKK

SEQ ID NO: 18 - protein, 5 aa, synthetic construct
KKKRK

SEQ ID NO: 19 - protein, 5 aa, synthetic construct
RKKKK

SEQ ID NO: 20 - protein, 5 aa, synthetic construct
KRKKK

SEQ ID NO: 21 - protein, 5 aa, synthetic construct
KKRKK

SEQ ID NO: 22 - protein, 5 aa, synthetic construct
KKKKR

SEQ ID NO: 23 - protein, 5 aa, synthetic construct
KBKBK

SEQ ID NO: 24 - protein, 6 aa, synthetic construct
RKKKKG

SEQ ID NO: 25 - protein, 6 aa, synthetic construct
KRKKKG

SEQ ID NO: 26 - protein, 6 aa, synthetic construct
KKRKKG

SEQ ID NO: 27 - protein, 6 aa, synthetic construct
KKKKRG

SEQ ID NO: 28 - protein, 6 aa, synthetic construct
RKKKKB

SEQ ID NO: 29 - protein, 5 aa, synthetic construct
HBKBH

SEQ ID NO: 30 - protein, 6 aa, synthetic construct
KRKKKB

SEQ ID NO: 31 - protein, 6 aa, synthetic construct
KKRKKB

SEQ ID NO: 32 - protein, 6 aa, synthetic construct
KKKKRB

SEQ ID NO: 33 - protein, 6 aa, synthetic construct
KKKRKV

SEQ ID NO: 34 - protein, 6 aa, synthetic construct
RRRRRR

SEQ ID NO: 35 - protein, 6 aa, synthetic construct
HHHHHH

SEQ ID NO: 36 - protein, 6 aa, synthetic construct
RHRHRH

SEQ ID NO: 37 - protein, 6 aa, synthetic construct
HRHRHR

SEQ ID NO: 38 - protein, 6 aa, synthetic construct
KRKRKR

SEQ ID NO: 39 - protein, 6 aa, synthetic construct
RKRKRK

SEQ ID NO: 40 - protein, 6 aa, synthetic construct
RBRBRB

SEQ ID NO: 41 - protein, 6 aa, synthetic construct
KBKBKB

SEQ ID NO: 42 - protein, 7 aa, synthetic construct
PKKKRKV

SEQ ID NO: 43 - protein, 7 aa, synthetic construct
PGKKRKV

SEQ ID NO: 44 - protein, 7 aa, synthetic construct
PKGKRKV

SEQ ID NO: 45 - protein, 7 aa, synthetic construct
PKKGRKV

SEQ ID NO: 46 - protein, 7 aa, synthetic construct
PKKKGKV

SEQ ID NO: 47 - protein, 7 aa, synthetic construct
PKKKRGV

SEQ ID NO: 48 - protein, 7 aa, synthetic construct
PKKKRKG

SEQ ID NO: 49 - protein, 5 aa, synthetic construct
RBRBR

SEQ ID NO: 50 - protein, 5 aa, synthetic construct
RBHBR

SEQ ID NO: 51 - protein, 5 aa, synthetic construct
HBRBH

SEQ ID NO: 52 - protein, 19 aa, synthetic construct
NLSKRPAAIKKAGQAKKKK

SEQ ID NO: 53 - protein, 9 aa, synthetic construct
PAAKRVKLD

SEQ ID NO: 54 - protein, 11 aa, synthetic construct
RQRRNELKRSF

SEQ ID NO: 55 - protein, 27 aa, synthetic construct
RMRKFKNKGKDTAELRRRRVEVSVELR

SEQ ID NO: 56 - protein, 14 aa, synthetic construct
KAKKDEQILKRRNV

SEQ ID NO: 57 - protein, 8 aa, synthetic construct
VSRKRPRP

SEQ ID NO: 58 - protein, 8 aa, synthetic construct
PPKKARED

SEQ ID NO: 59 - protein, 8 aa, synthetic construct
PQPKKKPL

SEQ ID NO: 60 - protein, 12 aa, synthetic construct
SALIKKKKKMAP

SEQ ID NO: 61 - protein, 5 aa, synthetic construct
DRLRR

SEQ ID NO: 62 - protein, 7 aa, synthetic construct
PKQKKRK

SEQ ID NO: 63 - protein, 10 aa, synthetic construct
RKLKKKIKKL

SEQ ID NO: 64 - protein, 10 aa, synthetic construct
REKKKFLKRR

SEQ ID NO: 65 - protein, 20 aa, synthetic construct
KRKGDEVDGVDEVAKKKSKK

SEQ ID NO: 66 - protein, 17 aa, synthetic construct
RKCLQAGMNLEARKTKK

SEQ ID NO: 67 - protein, 6 aa, synthetic construct
  SITE 2: note = X = L-3-(2-naphthyl)-alanine
FXRRRQ

SEQ ID NO: 68 - protein, 6 aa, synthetic construct
  SITE 2: note = X = L-3-(2-naphthyl)-alanine
FXRRRC

SEQ ID NO: 69 - protein, 6 aa, synthetic construct
  SITE 2: note = X = L-3-(2-naphthyl)-alanine
FXRRRU

SEQ ID NO: 70 - protein, 6 aa, synthetic construct
  SITE 4: note = X= L-3-(2-naphthyl)-alanine
RRRXFQ

SEQ ID NO: 71 - protein, 6 aa, unidentified
  SITE 5: note = X = L-3-(2-naphthyl)-alanine
RRRRXF

SEQ ID NO: 72 - protein, 6 aa, synthetic construct
  SITE 2: note = X = L-3-(2-naphthyl)-alanine
FXRRRR

SEQ ID NO: 73 - protein, 7 aa, synthetic construct
  SITE 2: note = X = D-3-(2-naphthyl)-alanine
FXRRRRQ

SEQ ID NO: 74 - protein, 6 aa, synthetic construct
  SITE 2: note = X = D-3-(2-naphthyl)-alanine
FXRRRR

SEQ ID NO: 75 - protein, 7 aa, synthetic construct
  SITE 2: note = X = L-3-(2-naphthyl)-alanine
FXRRRRQ

SEQ ID NO: 76 - protein, 7 aa, synthetic construct
  SITE 2: note = X = L-3-(2-naphthyl)-alanine
FXRRRRQ

SEQ ID NO: 77 - protein, 7 aa, synthetic construct
  SITE 5: note = X = L-3-(2-naphthyl)-alanine
RRFRXRQ

SEQ ID NO: 78 - protein, 7 aa, synthetic construct
  SITE 6: note = X = L-3-(2-naphthyl)-alanine
FRRRRXQ

SEQ ID NO: 79 - protein, 7 aa, synthetic construct
  SITE 5: note = X = L-3-(2-naphthyl)-alanine
RRFRXRQ

SEQ ID NO: 80 - protein, 7 aa, synthetic construct
  SITE 3: note = X = L-3-(2-naphthyl)-alanine
RRXFRRQ

SEQ ID NO: 81 - protein, 8 aa, synthetic construct
CRRRRFWQ

SEQ ID NO: 82 - protein, 8 aa, synthetic construct
  SITE 3: note = X = L-3-(2-naphthyl)-alanine
FFXRRRRQ

SEQ ID NO: 83 - protein, 8 aa, synthetic construct
  SITE 3: note = X = L-3-(2-naphthyl)-alanine
FFXRRRRQ

SEQ ID NO: 84 - protein, 8 aa, synthetic construct
  SITE 6: note = X = L-3-(2-naphthyl)-alanine
RFRFRXRQ

SEQ ID NO: 85 - protein, 8 aa, synthetic construct
URRRRFWQ

SEQ ID NO: 86 - protein, 8 aa, synthetic construct
CRRRRFWQ

SEQ ID NO: 87 - protein, 8 aa, synthetic construct
  SITE 2: note = X = L-3-(2-naphthyl)-alanine
FXRRRRQK

SEQ ID NO: 88 - protein, 8 aa, synthetic construct
  SITE 2: note = X = L-3-(2-naphthyl)-alanine
FXRRRRQC

SEQ ID NO: 89 - protein, 8 aa, synthetic construct
  SITE 2: note = X = L-3-(2-naphthyl)-alanine
FXRRRRRQ

SEQ ID NO: 90 - protein, 8 aa, synthetic construct
  SITE 2: note = X = L-3-(2-naphthyl)-alanine
FXRRRRRQ

SEQ ID NO: 91 - protein, 9 aa, synthetic construct
  SITE 5: note = X = L-3-(2-naphthyl)-alanine
  SITE 8: note = X = L-norleucine
RRRRXFDXC

SEQ ID NO: 92 - protein, 5 aa, synthetic construct
  SITE 2: note = X = L-3-(2-naphthyl)-alanine
FXRRR

SEQ ID NO: 93 - protein, 5 aa, synthetic construct
FWRRR

SEQ ID NO: 94 - protein, 5 aa, synthetic construct
  SITE 4: note = X = L-3-(2-naphthyl)-alanine
RRRXF

SEQ ID NO: 95 - protein, 5 aa, synthetic construct
RRRWF

SEQ ID NO: 96 - protein, 7 aa, synthetic construct
FGFGRGR

SEQ ID NO: 97 - protein, 7 aa, synthetic construct
GFFGRGR

SEQ ID NO: 98 - protein, 7 aa, synthetic construct
  SITE 2: note = X = L-3-(2-naphthyl)-alanine
FXFGRGR

SEQ ID NO: 99 - protein, 7 aa, synthetic construct
FFFGRGR

SEQ ID NO: 100 - protein, 7 aa, synthetic construct
  SITE 3: note = X = L-3-(2-naphthyl)-alanine
FFXGRGR

SEQ ID NO: 101 - protein, 8 aa, synthetic construct
FGFGRGRQ

SEQ ID NO: 102 - protein, 8 aa, synthetic construct
GFFGRGRQ

SEQ ID NO: 103 - protein, 8 aa, synthetic construct
  SITE 3: note = X = L-3-(2-naphthyl)-alanine
FFXGRGRQ

SEQ ID NO: 104 - protein, 8 aa, synthetic construct
FFFGRGRQ

SEQ ID NO: 105 - protein, 8 aa, synthetic construct
  SITE 3: note = X = L-3-(2-naphthyl)-alanine
FFXGRGRQ

SEQ ID NO: 106 - protein, 8 aa, synthetic construct
  SITE 3: note = X = L-3-(2-naphthyl)-alanine
FFXRRRRQ

SEQ ID NO: 107 - protein, 8 aa, synthetic construct
  SITE 3: note = X = L-3-(2-naphthyl)-alanine
  SITE 4: note = X = Citrulline
  SITE 6: note = X = Citrulline
FFXXRXRQ

SEQ ID NO: 108 - protein, 8 aa, synthetic construct
FGFGRRRQ

SEQ ID NO: 109 - protein, 8 aa, synthetic construct
FGFRRRRQ

SEQ ID NO: 110 - protein, 8 aa, synthetic construct
  SITE 3: note = X = L-3-(2-naphthyl)-alanine
FFXRGRGQ

SEQ ID NO: 111 - protein, 10 aa, synthetic construct
  SITE 3: note = X = L-3-(2-naphthyl)-alanine
FFXGPRGPRQ

SEQ ID NO: 112 - protein, 8 aa, synthetic construct
  SITE 3: note = X = L-3-(2-naphthyl)-alanine
FFXXRXRQ

SEQ ID NO: 113 - protein, 8 aa, synthetic construct
  SITE 3: note = X = L-3-(2-naphthyl)-alanine
FFXXRXRQ

SEQ ID NO: 114 - protein, 7 aa, synthetic construct
  SITE 2: note = X = L-3-(2-naphthyl)-alanine
  SITE 4: note = X = Citrulline
  SITE 6: note = X = Citrulline
FXRXRXQ

SEQ ID NO: 115 - protein, 8 aa, synthetic construct
PKKKRKVK

SEQ ID NO: 116 - protein, 8 aa, synthetic construct
  SITE 3: note = X = L-3-(2-naphthyl)-alanine
  SITE 6: note = X = Citrulline
FFXRRXRQ

SEQ ID NO: 117 - protein, 8 aa, synthetic construct
  SITE 3: note = X = L-3-(2-naphthyl)-alanine
  SITE 4: note = X = Citrulline
FFXXRRRQ

SEQ ID NO: 118 - protein, 8 aa, synthetic construct
  SITE 2: note = X = L-3-(2-naphthyl)-alanine
  SITE 5: note = X = Citrulline
  SITE 7: note = X = Citrulline
FFXRXRXQ

SEQ ID NO: 119 - protein, 7 aa, synthetic construct
  SITE 5: note = X = Citrulline
PKKKXKV

SEQ ID NO: 120 - protein, 12 aa, synthetic construct
PKKKRKVAEEAK

SEQ ID NO: 121 - protein, 5 aa, synthetic construct
RBFBR

SEQ ID NO: 122 - protein, 5 aa, synthetic construct
RBHBH

SEQ ID NO: 123 - protein, 8 aa, synthetic construct
  SITE 3: note = X = L-3-(2-naphthyl)-alanine
FFXSRSRQ

SEQ ID NO: 124 - protein, 8 aa, synthetic construct
FGFKRKRQ

SEQ ID NO: 125 - protein, 8 aa, synthetic construct
FGFRGRGQ

SEQ ID NO: 126 - protein, 10 aa, synthetic construct
FGFGRGRGRQ

SEQ ID NO: 127 - protein, 8 aa, synthetic construct
FGFGRRRQ

SEQ ID NO: 128 - protein, 8 aa, synthetic construct
FGFGRRRQ

SEQ ID NO: 129 - protein, 8 aa, synthetic construct
FGFRRRRQ

SEQ ID NO: 130 - protein, 4 aa, synthetic construct
RRRQ

SEQ ID NO: 131 - protein, 6 aa, synthetic construct
FGRRRQ

SEQ ID NO: 132 - protein, 6 aa, synthetic construct
FDRRRQ

SEQ ID NO: 133 - protein, 6 aa, synthetic construct
  SITE 2: note = X = 3-(3-benzothienyl)-alanine
FXRRRQ

SEQ ID NO: 134 - protein, 7 aa, synthetic construct
  SITE 2: note = X= L-3-(2-naphthyl)-alanine
FXRRRQF

SEQ ID NO: 135 - protein, 7 aa, synthetic construct
  SITE 2: note = X = L-3-(2-naphthyl)-alanine
  SITE 7: note = X = D-3-(2-naphthyl)-alanine
FXRRRQX

SEQ ID NO: 136 - protein, 7 aa, synthetic construct
  SITE 2: note = X = L-3-(2-naphthyl)-alanine
FXRRRQR

SEQ ID NO: 137 - protein, 7 aa, synthetic construct
FFFRRRQ

SEQ ID NO: 138 - protein, 7 aa, synthetic construct
FRFRFRQ

SEQ ID NO: 139 - protein, 7 aa, synthetic construct
  SITE 3: note = X = L-3-(2-naphthyl)-alanine
FFXRRRQ

SEQ ID NO: 140 - protein, 7 aa, synthetic construct
  SITE 2: note = X= L-3-(2-naphthyl)-alanine
FXFRRRQ

SEQ ID NO: 141 - protein, 7 aa, synthetic construct
FFFRRRQ

SEQ ID NO: 142 - protein, 7 aa, synthetic construct
  SITE 3: note = X = L-3-(2-naphthyl)-alanine
FFXRRRQ

SEQ ID NO: 143 - protein, 7 aa, unidentified
  SITE 2: note = X =L-3-(2-naphthyl)-alanine
FXFRRRQ

SEQ ID NO: 144 - protein, 7 aa, synthetic construct
  SITE 1: note = X = D-3-(2-naphthyl)-alanine
XFFRRRQ

SEQ ID NO: 145 - protein, 8 aa, synthetic construct
FFFRRRRQ

SEQ ID NO: 146 - protein, 8 aa, synthetic construct
RFRFRFRQ

SEQ ID NO: 147 - protein, 8 aa, synthetic construct
FFFRRRRQ

SEQ ID NO: 148 - protein, 8 aa, synthetic construct
FFFRRRRQ

SEQ ID NO: 149 - protein, 8 aa, synthetic construct
FFFRRRRQ

SEQ ID NO: 150 - protein, 8 aa, synthetic construct
  SITE 3: note = X =L-3-(2-naphthyl)-alanine
FFXRRRAQ

SEQ ID NO: 151 - protein, 8 aa, synthetic construct
  SITE 3: note = X = L-3-(2-naphthyl)-alanine
FFXRRARQ

SEQ ID NO: 152 - protein, 8 aa, synthetic construct
  SITE 3: note = X= L-3-(2-naphthyl)-alanine
FFXRARRQ

SEQ ID NO: 153 - protein, 8 aa, synthetic construct
  SITE 3: note = X = L-3-(2-naphthyl)-alanine
FFXARRRQ

SEQ ID NO: 154 - protein, 8 aa, synthetic construct
FFARRRRQ

SEQ ID NO: 155 - protein, 6 aa, unidentified
  SITE 2: note = X = D-3-(2-naphthyl)-alanine
FXRRRQ

SEQ ID NO: 156 - protein, 5 aa, synthetic construct
  SITE 2: note = X= D-3-(2-naphthyl)-alanine
FXRRQ

SEQ ID NO: 157 - protein, 6 aa, synthetic construct
FFRRRQ

SEQ ID NO: 158 - protein, 7 aa, synthetic construct
FFFRRRQ

SEQ ID NO: 159 - protein, 8 aa, synthetic construct
  SITE 3: note = X = L-3-(2-naphthyl)-alanine
FAXRRRRQ

SEQ ID NO: 160 - protein, 8 aa, synthetic construct
  SITE 3: note = X = L-3-(2-naphthyl)-alanine
AFXRRRRQ

SEQ ID NO: 161 - protein, 5 aa, synthetic construct
RRRRQ

SEQ ID NO: 162 - protein, 10 aa, synthetic construct
FFXXXRRRRQ

SEQ ID NO: 163 - protein, 8 aa, synthetic construct
FFXRRRRQ

SEQ ID NO: 164 - protein, 6 aa, synthetic construct
  SITE 3: note = X = L- 3-(2-naphthyl)-alanine
FFXRRQ

SEQ ID NO: 165 - protein, 6 aa, synthetic construct
  SITE 1: note = X = L- 3-(2-naphthyl)-alanine
XRRRRQ

SEQ ID NO: 166 - protein, 9 aa, synthetic construct
  SITE 3: note = X = L- 3-(2-naphthyl)-alanine
FFXRRRRQF

SEQ ID NO: 167 - protein, 9 aa, synthetic construct
  SITE 3: note = X = L- 3-(2-naphthyl)-alanine
  SITE 9: note = X = L- 3-(2-naphthyl)-alanine
FFXRRRRQX

SEQ ID NO: 168 - protein, 9 aa, synthetic construct
  SITE 3: note = X = L- 3-(2-naphthyl)-alanine
  SITE 9: note = X = D- 3-(2-naphthyl)-alanine
FFXRRRRQX

SEQ ID NO: 169 - protein, 9 aa, synthetic construct
  SITE 3: note = X = L- 3-(2-naphthyl)-alanine
FFXRRRRQR

SEQ ID NO: 170 - protein, 9 aa, synthetic construct
  SITE 3: note = X = L- 3-(2-naphthyl)-alanine
FFXRRRRNF

SEQ ID NO: 171 - protein, 8 aa, synthetic construct
  SITE 3: note = X = L- 3-(2-naphthyl)-alanine
FFXRRRRQ

(19) United States
(12) Patent Application Publication
(10) Pub. No.: US 2025/0289851 A1
(43) Pub. Date: Sep. 18, 2025

(54) CYCLIC PEPTIDES FOR DELIVERING THERAPEUTICS

(71) Applicant: ENTRADA THERAPEUTICS, INC., Boston, MA (US)

(72) Inventors: Patrick Dougherty, Independence, OH (US); Kyle A. Totaro, Attleboro, MA (US); Amanda Dombroski, Holbrook, MA (US); Riley Giesler, Boston, MA (US); Ming Zhou, Coppell, TX (US); Matthew Streeter, Weymouth, MA (US); Alec Goffin, Great Barrington, MA (US); Ziqing Qian, Lexington, MA (US)

(21) Appl. No.: 18/858,718

(22) PCT Filed: Apr. 21, 2023
(86) PCT No.: PCT/US2023/019452

§ 371 Date: Oct. 21, 2024

Related U.S. Application Data
(60) Provisional application No. 63/363,450, filed on Apr. 22, 2022.
(60) Provisional application No. 63/354,471, filed on Jun. 22, 2022.
(60) Provisional application No. 63/377,754, filed on Sep. 30, 2022.

(51) Int. Cl. C07K 7/64; A61K 47/64; C07K 7/06

(52) U.S. Cl. CPC C07K 7/64; A61K 47/64; C07K 7/06

(57) ABSTRACT

The present disclosure relates to the synthesis of cyclic peptides that are able to effectively deliver cargo, e.g., a therapeutic moiety (TM), inside a cell to treat a variety of conditions and diseases.

This application claims benefit of priority to the filing dates of U.S. Provisional Application Ser. No. 63/363,450 filed Apr. 22, 2022, U.S. Provisional Application Ser. No. 63/354,471 filed Jun. 22, 2022, and U.S. Provisional Application Ser. No. 63/377,754 filed Sep. 30, 2022, the contents of which are specifically incorporated herein by reference in their entireties

### BACKGROUND

Nucleic acids and their synthetic analogs hold enormous potential as therapeutic agents, especially against targets that are challenging for conventional drug modalities (e.g., missing/defective proteins caused by genetic mutations).

However, one challenge in translating the potential of such therapies to the clinic is their limited ability to gain access to the intracellular compartment when administered systemically. Carrier systems, such as polymers, cationic liposomes or chemical modifications, for example by the covalent attachment of cholesterol molecules, have been used facilitate intracellular delivery. Still, intracellular delivery efficiency by these approaches is often low and improved delivery systems to increase efficacy of intracellular delivery have remained elusive.

Effective compositions to deliver therapeutic molecules to intracellular compartments in order to treat disease are needed and some have recently been reported. However, efficient synthesis of such compounds remains needed.

The disclosure addresses these and other issues.

### SUMMARY

The disclosure relates to a method of making a cyclic peptide of formula (A):

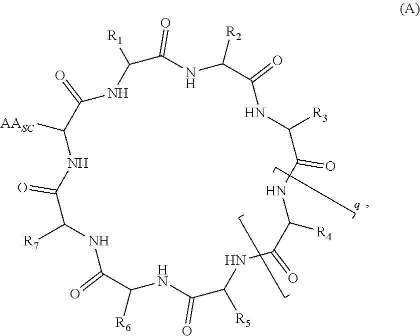

or a protonated form thereof,
wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl) alanine;
   AA_{SC} is an amino acid side chain; and
q is 1, 2, 3 or 4;
wherein the method is any one of the methods herein described for a compound of Formula (I).

The disclosure relates to a method of making a cyclic peptide of Formula (I):

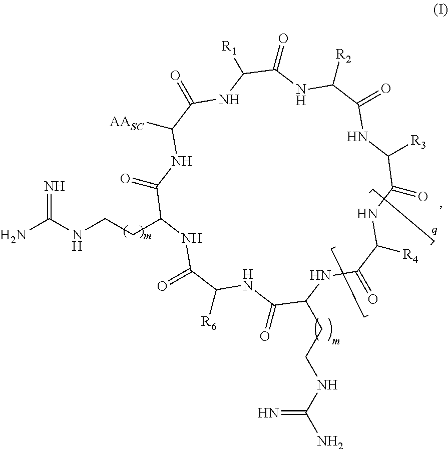

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3;
   the method comprising:
reacting a compound of formula (1)

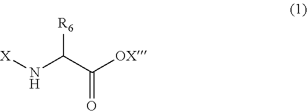

with a compound of formula (II)

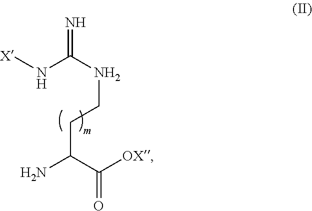

to form a compound of formula (III)

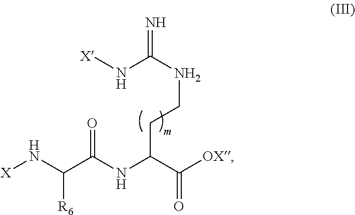

wherein X, and X′ are independently protecting groups, X″ is H or a protecting group, and X′″ is H or an activating group (e.g., NHS ester) and m is 0-3.

The disclosure also relates to a method of making a cyclic peptide of Formula (I):

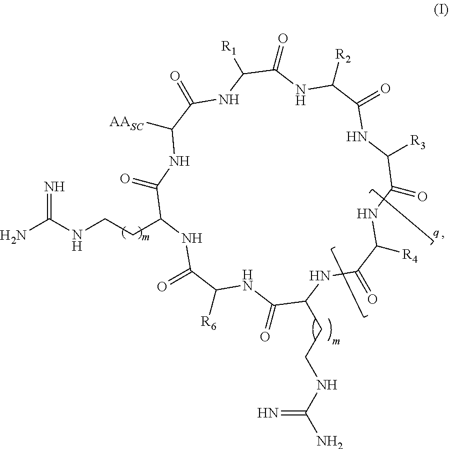

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
   each m is independently an integer 0, 1, 2, or 3,
   the method comprising:
   reacting a compound of formula (IX)

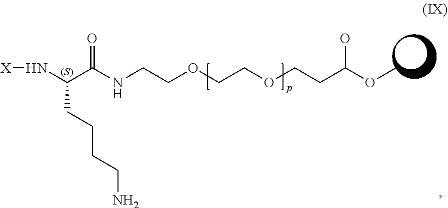

wherein

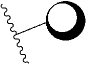

is a solid support, with a compound of formula (X)

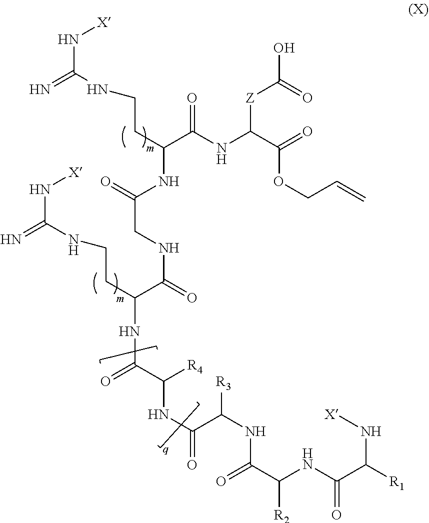

wherein X and X′ are independently protecting groups and Z is a radical of an amino acid side chain, to form a compound of formula (XI)

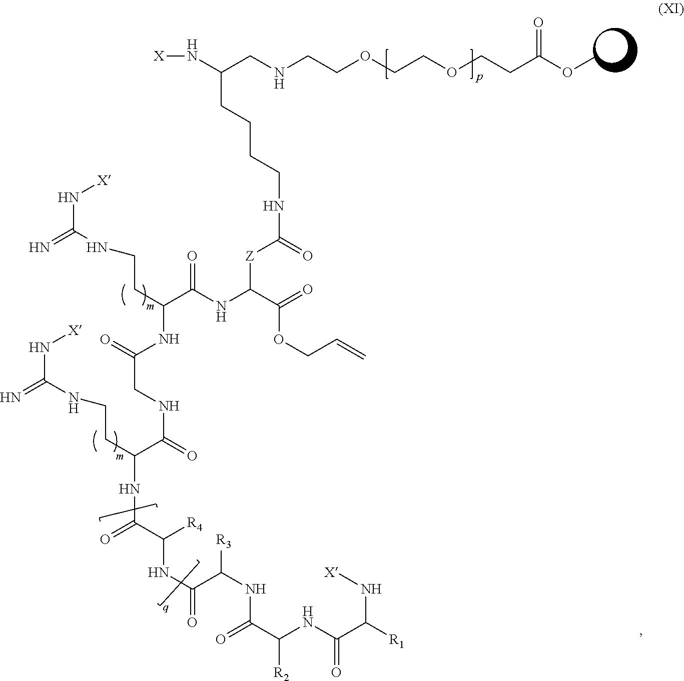

wherein p an integer from 1-30.

The disclosure relates to a method of making a cyclic peptide of Formula (I):

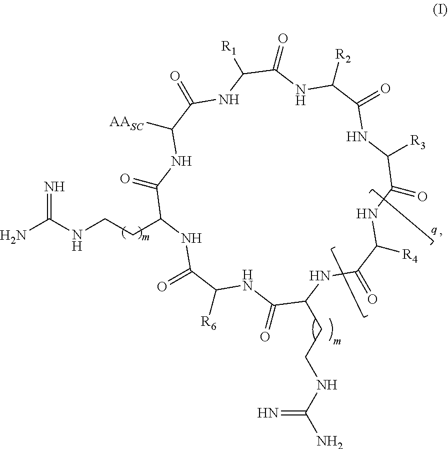

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3,
   the method comprising: reacting a compound of formula (XIII)

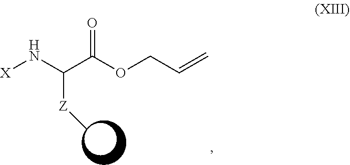

wherein wherein X is a protecting group and Z is a radical of an amino acid side chain and

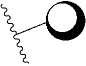

is a solid support, with a compound of formula (XIV)

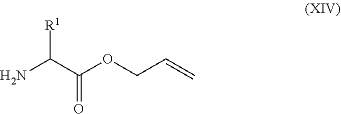

to give a compound of formula (XV)

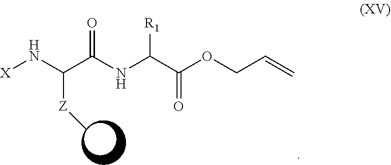

The disclosure relates to a compound selected from

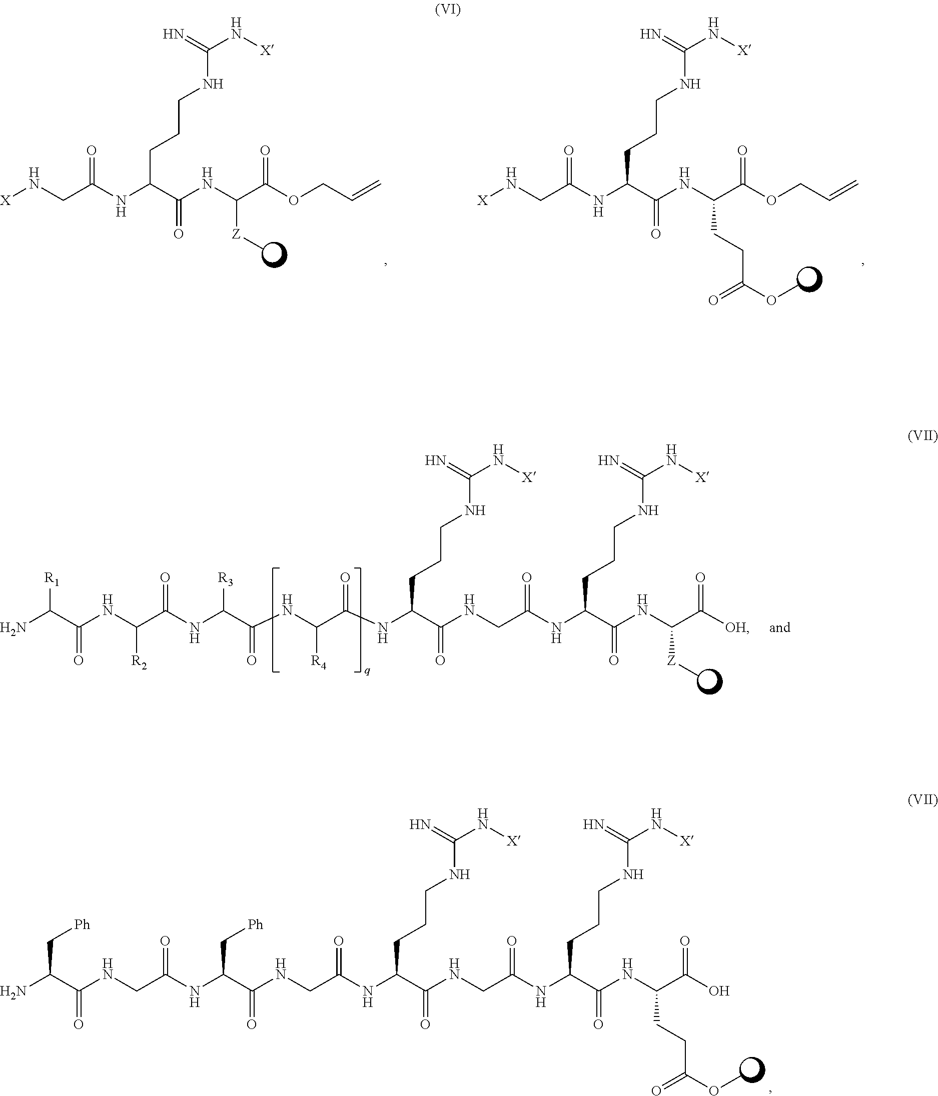

wherein:
R₁, R₂, and R₃ are each independently H or a side chain of tyrosine, phenylalanine or tryptophan;
R₄ is H or an amino acid side chain;
Z is a radical of an amino acid side chain;
q is 1, 2, 3 or 4;
X, X′, and X″ are each independently protecting groups;
each m is independently an integer from 0-3; and

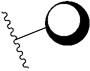

is a solid support.

The disclosure relates to compound selected from

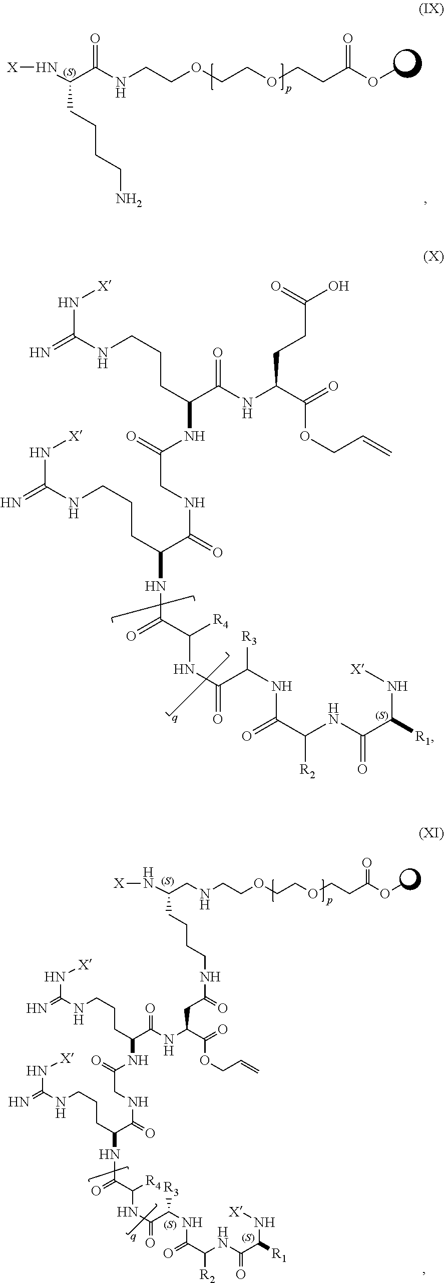

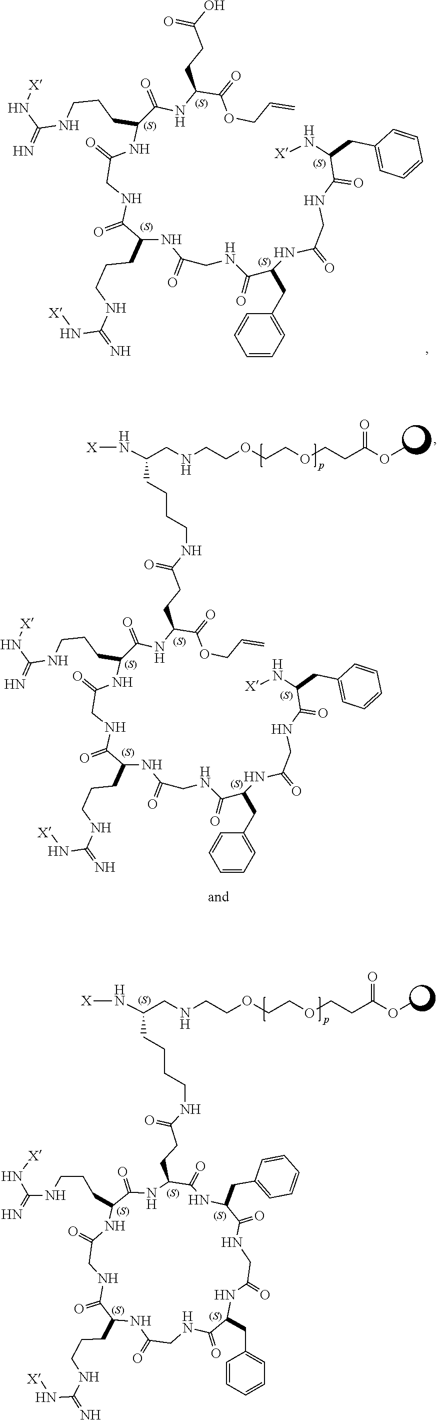

wherein:
R₁, R₂, and R₃ are each independently H or a side chain of tyrosine, phenylalanine or tryptophan;
R₄ is H or an amino acid side chain;
p is an integer from 1-30;
q is 1, 2, 3 or 4
X and X″ are each independently protecting groups;
each m is independently an integer from 0-3; and

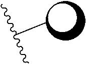

is a solid support.

The disclosure relates to compound selected from

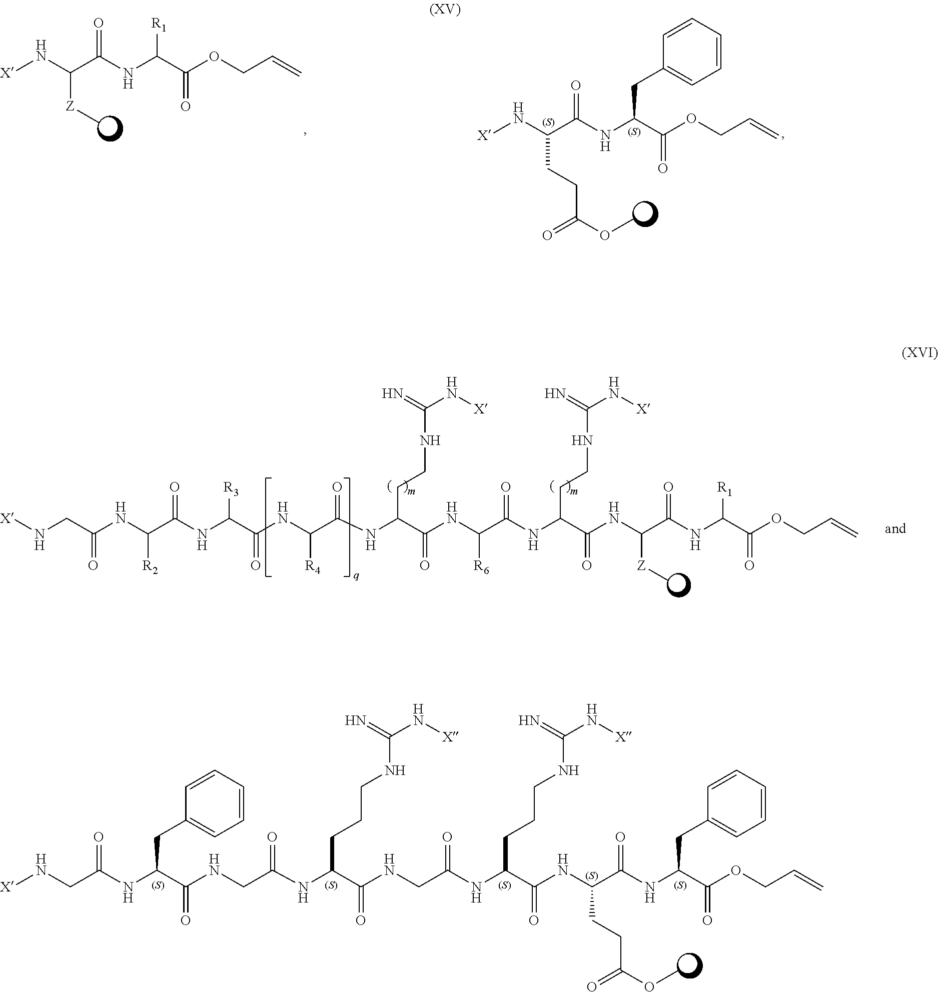

wherein:
R₁, R₂, and R₃ are each independently H or a side chain of tyrosine, phenylalanine or tryptophan;
R₄ is H or an amino acid side chain;
Z is a radical of an amino acid side chain;
q is 1, 2, 3 or 4;
X′, and X″ are each independently protecting groups;
each m is independently an integer from 0-3; and

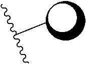

is a solid support.

### BRIEF DESCRIPTION OF THE FIGURES

FIGS. 1A and 1B show a synthetic route of making an EEV.

FIG. 2 shows a synthetic route of making a cyclic peptide.

FIG. 3 shows a synthetic route of making a cyclic peptide.

FIG. 4 shows a synthetic route of making a cyclic peptide.

FIG. 5 shows a synthetic route of making an EEV.

FIG. 6 shows a synthetic route of making an EEV.

FIG. 7 shows a synthetic route of making a cyclic peptide.

FIG. 8 shows a synthetic route of making an EEV.

FIG. 9 shows Generation 1 EEV-PMO synthesis, wherein PMO is Phosphorodiamidate Morpholino Oligomer.

FIG. 10 shows Generation 2 EEV-PMO synthesis.

### DETAILED DESCRIPTION

The disclosure relates to a method of making an endosomal escape vehicle (EEV).

### Endosomal Escape Vehicles (EEVs)

An endosomal escape vehicle (EEV) is provided herein that can be used to transport cargo across a cellular membrane, for example, to deliver the cargo to the cytosol or nucleus of a cell. Cargo can include a macromolecule, for example, a peptide or oligonucleotide, or a small molecule. The EEV can comprise a cell penetrating peptide (CPP), for example, a cyclic cell penetrating peptide (cCPP), which is conjugated to an exocyclic peptide (EP). The EP can comprise a sequence of a nuclear localization signal (NLS). The EP can be coupled to the cargo. The EP can be coupled to the cCPP. The EP can be coupled to the cargo and the cCPP. Coupling between the EP, cargo, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an oligonucleotide cargo. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the cargo.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 4-methyltrityl (Mtt), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct a cargo (e.g., a therapeutic moiety (TM) such as an oligonucleotide, peptide or small molecule) to penetrate the membrane of a cell. The cCPP can deliver the cargo to the cytosol of the cell. The cCPP can deliver the cargo to a cellular location where a target (e.g., pre-mRNA) is located. To conjugate the cCPP to a cargo (e.g., peptide, oligonucleotide, or small molecule), at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

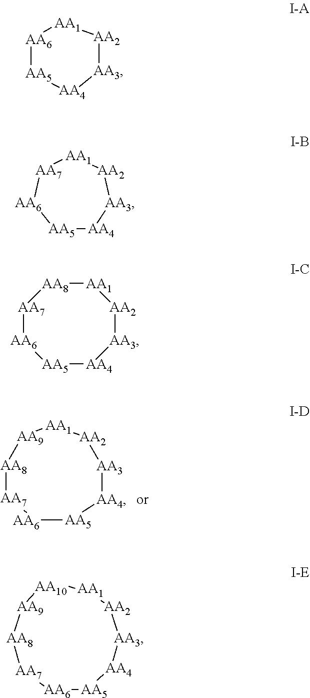

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbrevi- | Abbrevi- |
|---|---|---|
| | ations* | ations* |
| | L-amino | D-amino |
| Amino Acid | acid | acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine | Nal (Φ) | nal (ϕ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: when shown in capital letters herein it indicates the L-amino acid form, when shown in lower case herein it indicates the D-amino acid form.

The cCPP can comprise 4 to 20 amino acids, wherein: (i) at least one amino acid has a side chain comprising a guanidine group, or a protonated form thereof; (ii) at least one amino acid has no side chain or a side chain comprising

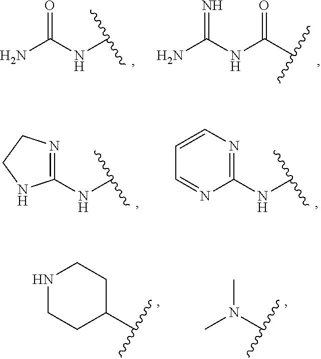

or a protonated form thereof; and (iii) at least two amino acids independently have a side chain comprising an aromatic or heteroaromatic group.

At least two amino acids can have no side chain or a side chain comprising

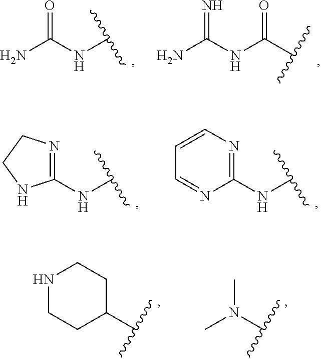

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, b-alanine, or 4-aminobutyric acid residues; (ii) at least one amino acid can have a side chain comprising an aryl or heteroaryl group; and (iii) at least one amino acid has a side chain comprising a guanidine group,

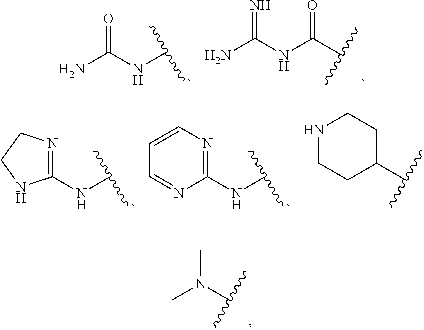

or a protonated form thereof.

The cCPP can comprise from 6 to 20 amino acid residues which form the cCPP, wherein: (i) at least two amino acid can independently beglycine, b-alanine, or 4-aminobutyric acid residues; (ii) at least one amino acid can have a side chain comprising an aryl or heteroaryl group; and (iii) at least one amino acid has a side chain comprising a guanidine group,

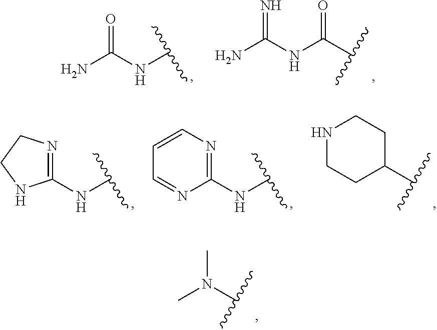

or a protonated form thereof.

The cCPP can comprise from 6 to 20 amino acid residues which form the cCPP, wherein: (i) at least three amino acids can independently be glycine, b-alanine, or 4-aminobutyric acid residues; (ii) at least one amino acid can have a side chain comprising an aromatic or heteroaromatic group; and (iii) at least one amino acid can have a side chain comprising a guanidine group,

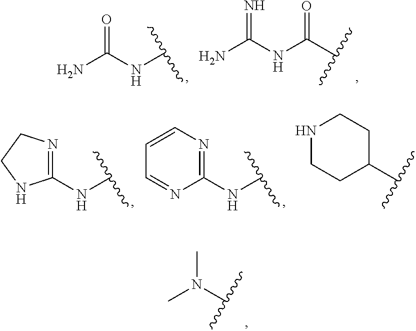

or a protonated form thereof.

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, b-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues.

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl) cysteine, N-(naphthalen-2-yl) glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

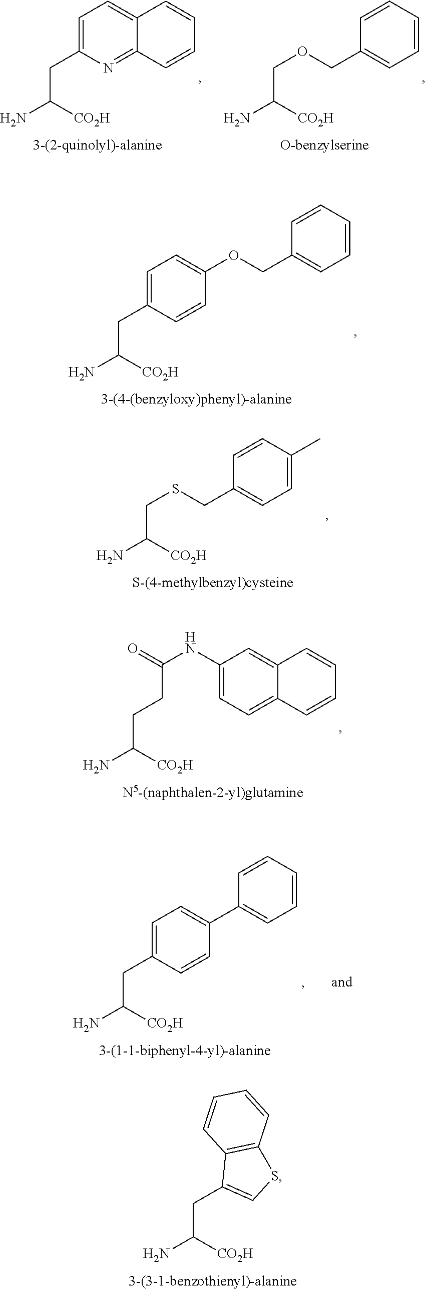

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent.

The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A 1984; 81 (1): 140-144), Engleman, et al. (Ann. Rev. of Biophys. Biophys. Chem. 1986; 1986 (15): 321-53), Kyte and Doolittle (J. Mol. Biol. 1982; 157 (1): 105-132), Hoop and Woods (Proc. Natl. Acad. Sci. U.S.A. 1981; 78 (6): 3824-3828), and Janin (Nature. 1979; 277 (5696): 491-492), the entirety of each of which is herein incorporated by reference. Hydrophobicity can be measured using the hydrophobicity scale reported in Engleman, et al.

TABLE 2
Amino Acid Hydrophobicity

| | | | | Kyrie | Hoop | |
|---|---|---|---|---|---|---|
| Amino | | Eisenberg | Engleman | and | and | |
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

### Amino Acid Residues Having a Side Chain Comprising a Guanidine Group, Guanidine Replacement Group, or Protonated Form Thereof

As used herein, guanidine refers to the structure:

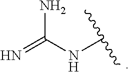 [SMILES: *NC(=N)N]

As used herein, a protonated form of guanidine refers to the structure:

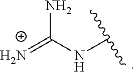 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

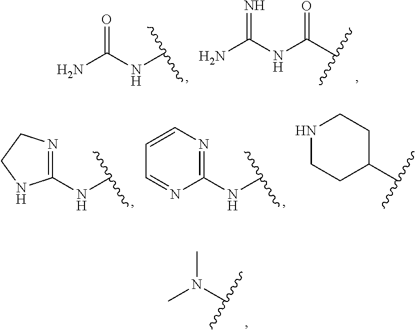

or a protonated form thereof.

The disclosure relates to a cCPP comprising from 4 to 20 amino acids residues, wherein: (i) at least one amino acid has a side chain comprising a guanidine group, or a protonated form thereof, (ii) at least one amino acid residue has no side chain or a side chain comprising

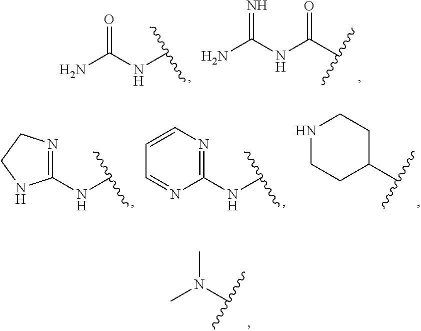

or a protonated form thereof; and (iii) at least two amino acids residues independently have a side chain comprising an aromatic or heteroaromatic group.

At least two amino acids residues can have no side chain or a side chain comprising

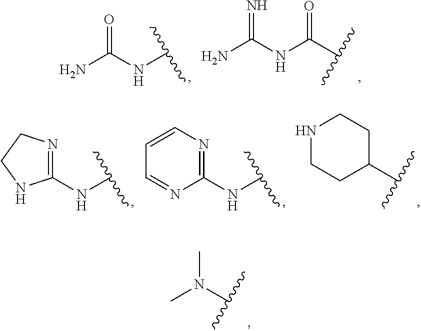

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

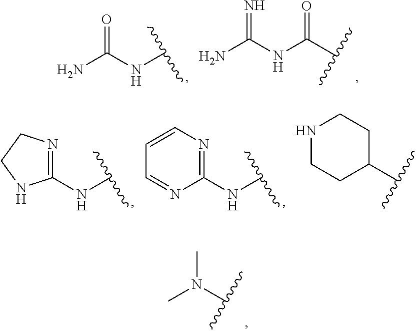

or a protonated form thereof.

The cCPP can comprise at least two amino acids each independently having one of the following moieties

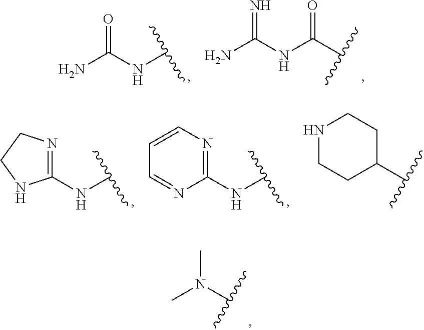

or a protonated form thereof. At least two amino acids can have a side chain comprising the same moiety selected from:

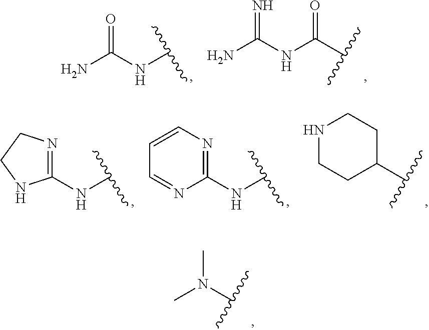

or a protonated form thereof. At least one amino acid can have a side chain comprising

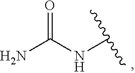 [SMILES: *NC(N)=O]

or a protonated form thereof. At least two amino acids can have a side chain comprising

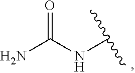 [SMILES: *NC(N)=O]

or a protonated form thereof. One, two, three, or four amino acids can have a side chain comprising

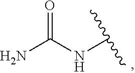 [SMILES: *NC(N)=O]

or a protonated form thereof. One amino acid can have a side chain comprising

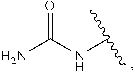 [SMILES: *NC(N)=O]

or a protonated form thereof. Two amino acids can have a side chain comprising

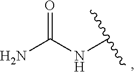 [SMILES: *NC(N)=O]

or a protonated form thereof.

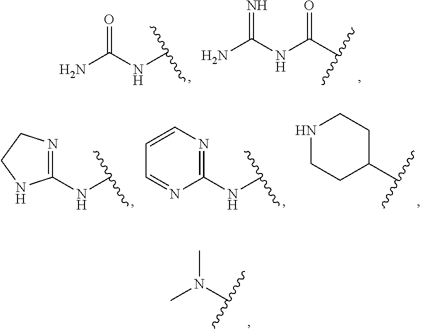

or a protonated form thereof, can be attached to the terminus of the amino acid side chain.

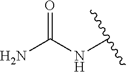 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

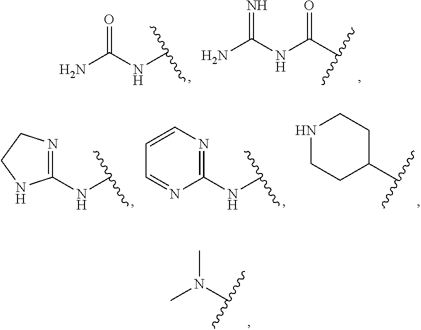

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutaminc acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid The cCPPs can comprise the following sequences: D/L-X-D/L; D/L-X-D/L-X; D/L-X-D/L-X-D/L; D-X-D; D-X-D-X; D-X-D-X-D; L-X-L; L-X-L-X; or L-X-L-X-L, wherein D/L means that the amino acid can have D or L stereochemistry and X is an achiral amino acid. The achiral amino acid can be glycine.

An amino acid having a side chain comprising:

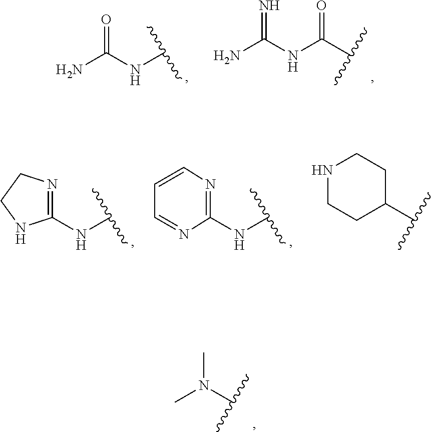

or a protonated form thereof, can be adjacent to an amino acid having a side chain comprising an aromatic or heteroaromatic group. An amino acid having a side chain comprising:

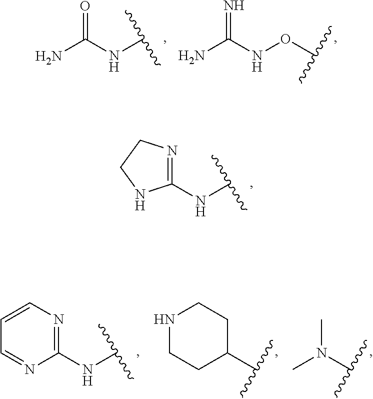

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

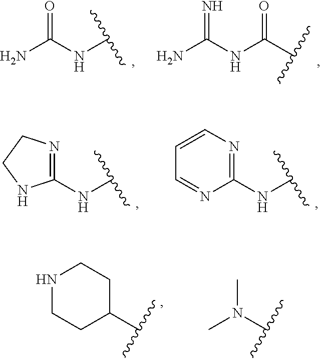

or protonated forms there, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

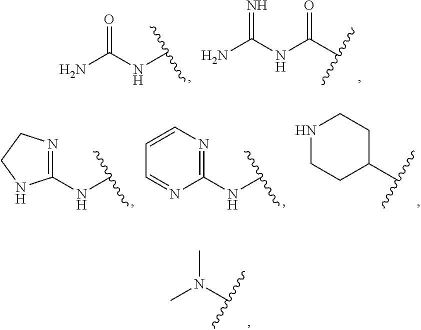

or a protonated form thereof. The cCPPs can comprise at least two contiguous amino acids having a side chain comprising an aromatic or heteroaromatic group and at least two non-adjacent amino acids having a side chain comprising

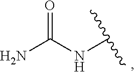 [SMILES: *NC(N)=O]

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

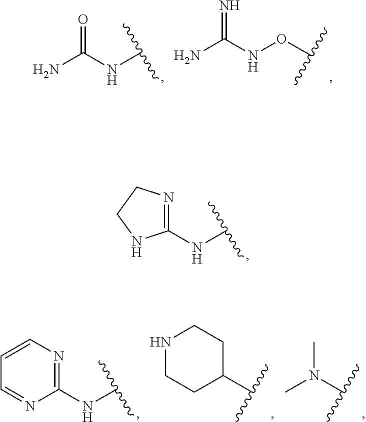

or a protonated form thereof, are alternating with at least two amino acids having a side chain comprising a guanidine group or protonated form thereof.

The cCPP can comprise the structure of Formula (A):

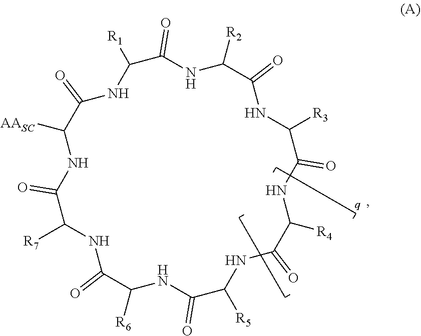

or a protonated form thereof,
wherein:
   R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl) alanine;
AA_{SC} is an amino acid side chain; and
q is 1, 2, 3 or 4.

In embodiments, at least one of R₄, R₅, R₆, R₇ are independently a uncharged, non-aromatic side chain of an amino acid. In embodiments, at least one of R₄, R₅, R₆, R₇ are independently H or a side chain of citrulline.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine) or a combination thereof. In embodiments, at least two uncharged, non-aromatic amino acids of the cyclic peptide are citrulline, glycine or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphtha alanine and combinations thereof, and at least two amino acids are uncharged, non-aromatic amino acids selected from citrulline, glycine and combinations thereof.

In embodiments, the cyclic peptide of Formula (A) is not a cyclic peptide having a sequence of:

CPP sequences

| | | |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | fΦRrRrRQ |
| RRRΦFQ | RRΦFRRQ | FΦRRRRRQ |
| RRRRΦF | CRRRRFWQ | RRRRΦFDQC |
| FΦRRRR | FfΦRrRrQ | FΦRRR |
| FϕrRrRq | FFΦRRRRQ | FWRRR |
| FϕrRrRQ | RFRFRΦRQ | RRRΦF |
| FΦRRRRQ | URRRRFWQ | RRRWF |
| fΦRrRrQ | CRRRRFWQ | |

where F is L-phenylalanine, f is D-phenylalanine, Φ is L-3-(2-naphthyl)-alanine, Φ is D-3-(2-naphthyl)-alanine, R is L-arginine, r is D-arginine, Q is L-glutamine, q is D-glutamine, C is L-cysteine, U is L-selenocysteine, W is L-tryptophan, K is L-lysine, D is L-aspartic acid, and Q is L-norleucine.

The cCPP can comprise the structure of Formula (I):

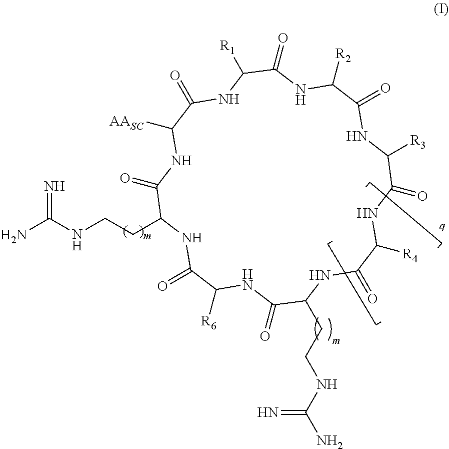

or a protonated form thereof,
   wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, β-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 1. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃alkylene-heteroaryl. R₆ can be H or -alkylene-aryl. R₆ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1. R₆ can be H or an amino acid residue having a side chain comprising an aromatic group. R₆ can be H, —CH₂Ph, or —CH₂ Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 1. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and Ry can be —CH₂Ph. At least one of R₁, R₂, R₃, R₄, R₅, R₆, and Ry can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and Ry can be H. Two of R₁, R₂, R₃, R₄, R₅, R₆, and R₇ are H. Three of R₁, R₂, R₃, R₅, R₆, and Ry can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, β-homoarginine. At least one of R₄, R₅, R₆, and R₇ can be side chain of 3-(1-piperidinyl) alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, β-homoarginine. At least two of R₄, R₅, R₆, and R₇ can be side chain of 3-(1-piperidinyl) alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, β-homoarginine. At least three of R₄, R₅, R₆, and R₇ can be side chain of 3-(1-piperidinyl) alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

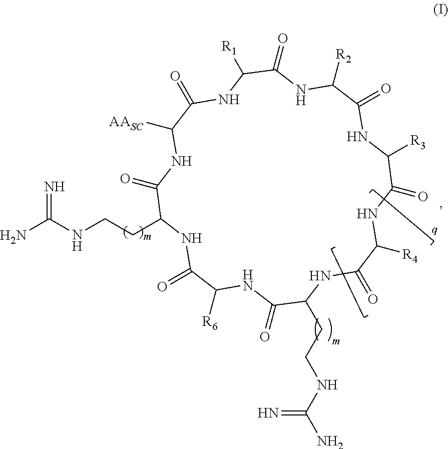

or protonated form thereof, wherein AA_{SC}, R₁, R₂, R₃, R₄, R₆, m and q are as defined herein.

The cCPP of Formula (A) can comprise the structure of Formula (I-a) or Formula (I-b):

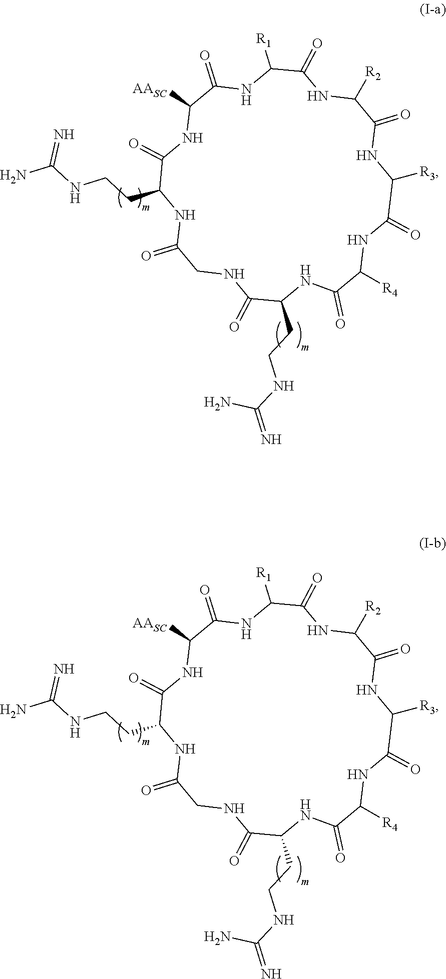

or protonated form thereof, wherein AA_{SC}, R₁, R₂, R₃, R₄, and m are as defined herein.

The cCPP of Formula (A) can comprise the structure of Formula (I-1), (I-2), (I-3) or (I-4):

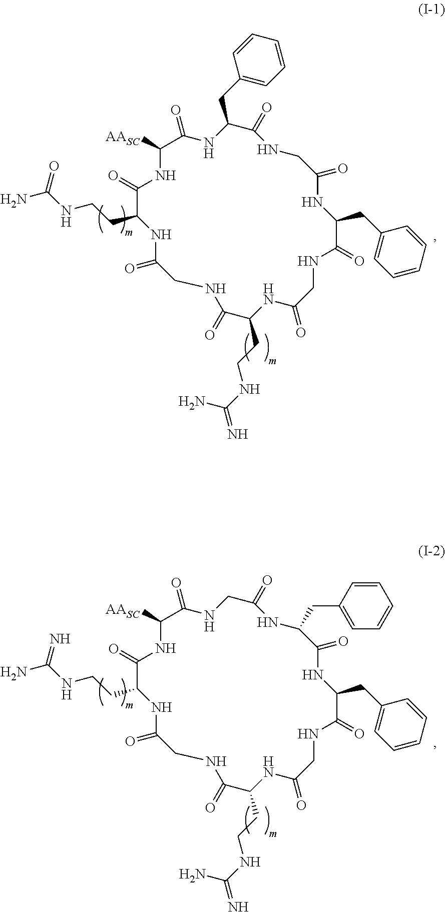

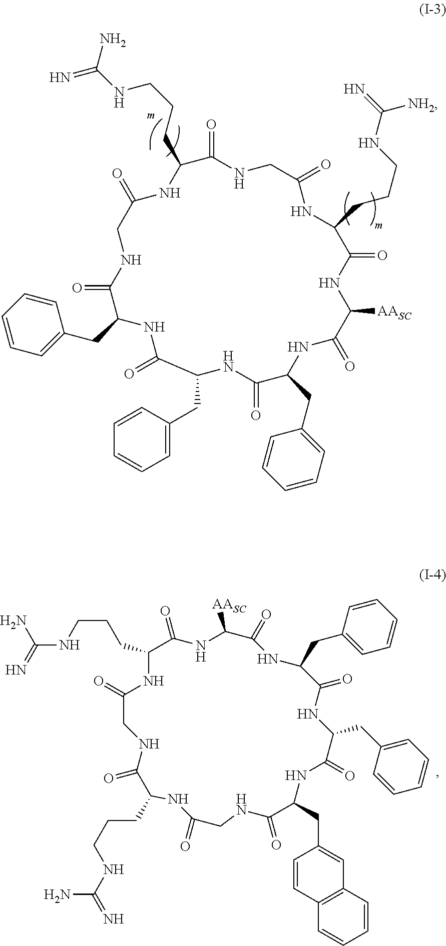

or protonated form thereof, wherein AA_{SC} and m are as defined herein.

The cCPP of Formula (A) can comprise the structure of Formula (I-5) or (I-6):

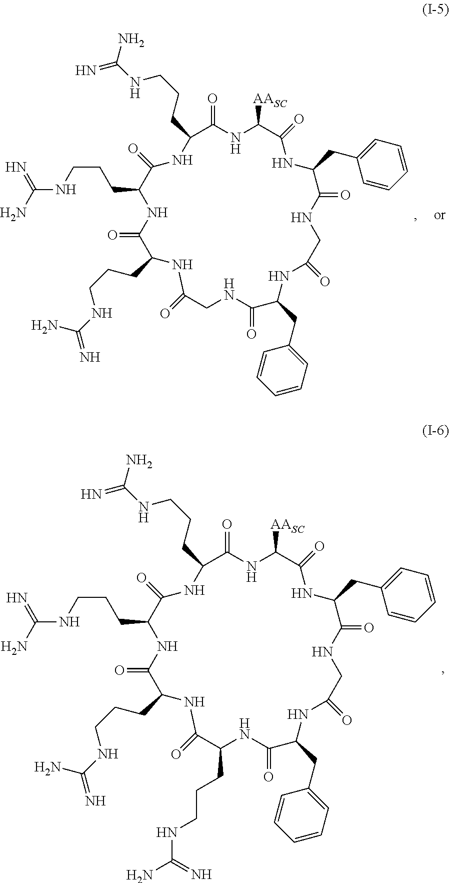

or protonated form thereof, wherein AA_{SC} is as defined herein.

The cCPP of Formula (A) can comprise the structure of Formula (I-1):

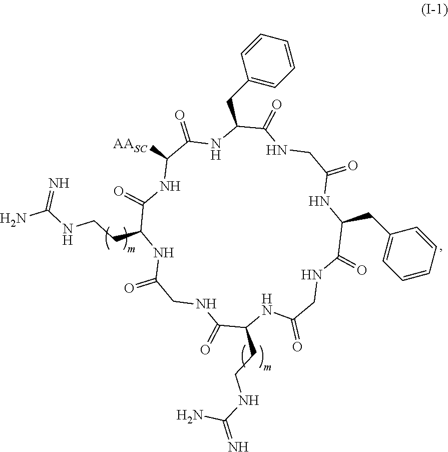

or a protonated form thereof,
wherein AA_{SC} and m are as defined herein.

The cCPP of Formula (A) can comprise the structure of Formula (I-2):

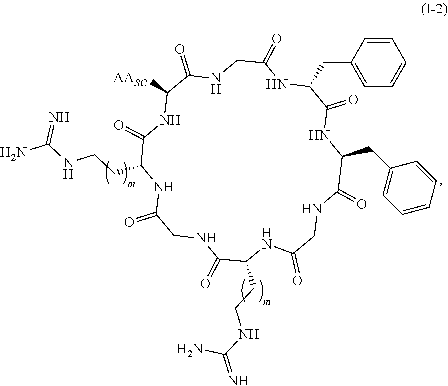

or a protonated form thereof, wherein AA_{SC} and m are as defined herein.

The cCPP of Formula (A) can comprise the structure of Formula (I-3):

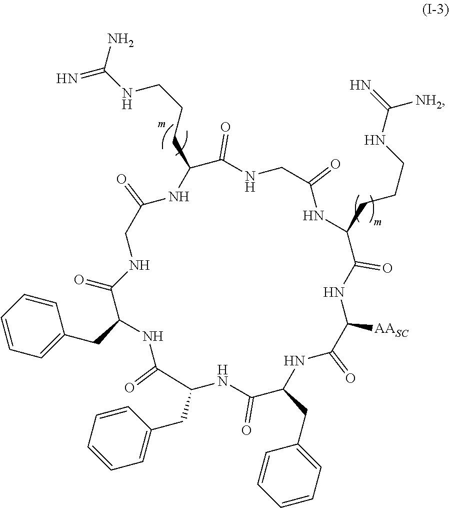

or a protonated form thereof, wherein AA_{SC} and m are as defined herein.

The cCPP of Formula (A) can comprise the structure of Formula (I-4):

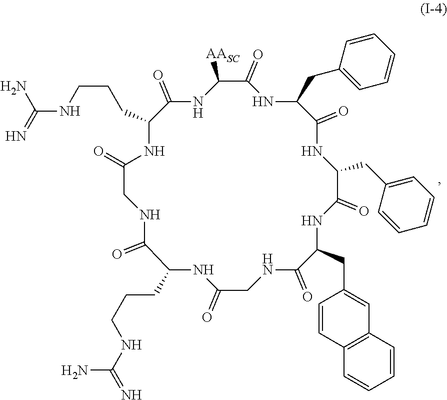

or a protonated form thereof, wherein AA_{SC} and m are as defined herein.

The cCPP of Formula (A) can comprise the structure of Formula (I-5):

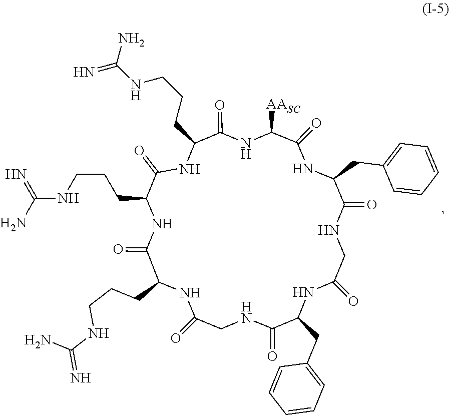

or a protonated form thereof, wherein AA_{SC} and m are as defined herein.

The cCPP of Formula (A) can comprise the structure of Formula (I-6):

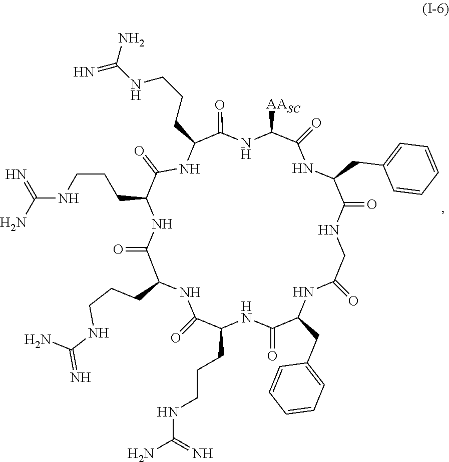

or a protonated form thereof, wherein AA_{SC} and m are as defined herein.

The cCPP can comprise one of the following sequences: FGFGRGR; GfFGrGr, FfΦGRGR; FfFGRGR; or FfΦGrGr. The cCPP can have one of the following sequences: FGFGRGRQ; GfFGrGrQ, FfΦGRGRQ; FfFGRGRQ; or FfΦGrGrQ.

The disclosure also relates to a cCPP having the structure of Formula (II):

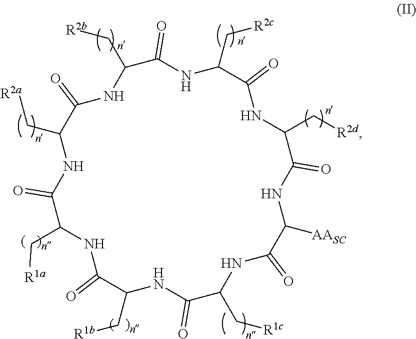

or protonated form thereof,
wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

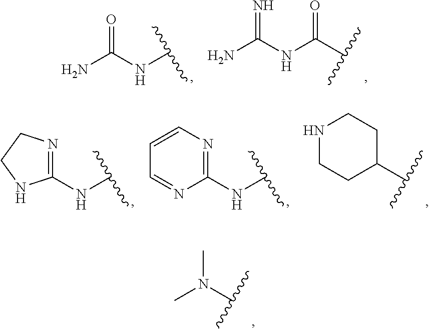

or a protonated form thereof;
at least one of R^{2a}, R^{2b}, R^{2c} and R^{2d} is guanidine or a protonated form thereof;
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

At least two of R^{2a}, R^{2b}, R^{2c} and R^{2d} can be

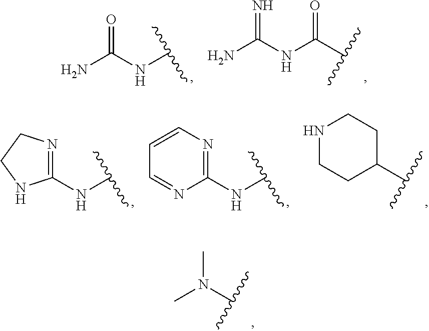

or a protonated form thereof. Two or three of R^{2a}, R^{2b}, R^{2c} and R^{2d} can be

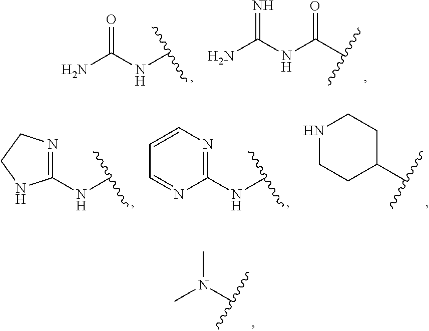

or a protonated form thereof. One of R^{2a}, R^{2b}, R^{2c} and R^{2d} can be

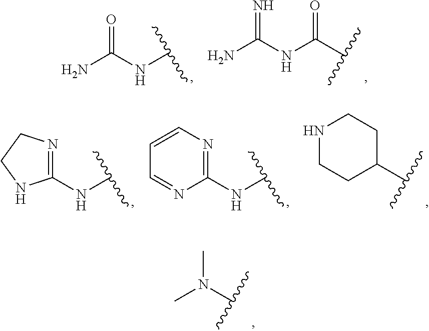

or a protonated form thereof. At least one of R^{2a}, R^{2b}, R^{2c} and R^{2d} can be

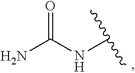 [SMILES: *NC(N)=O]

or a protonated form thereof, and the remaining of R^{2a}, R^{2b}, R^{2c} and R^{2d} can be guanidine or a protonated form thereof. At least two of R^{2a}, R^{2b}, R^{2c} and R^{2d} can be

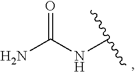 [SMILES: *NC(N)=O]

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

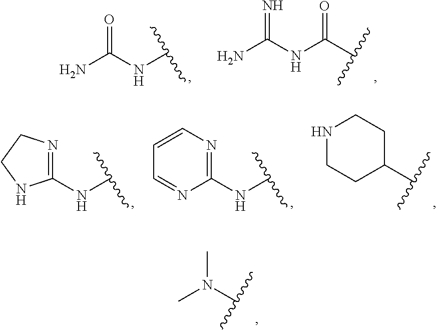

or a protonated form thereof. At least of R^{2a}, R^{2b}, R^{2c} and R^{2d} can be

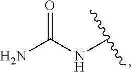 [SMILES: *NC(N)=O]

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

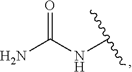 [SMILES: *NC(N)=O]

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

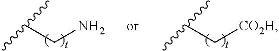

wherein t can be an integer from 0 to 5. AA_{SC} can be

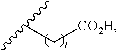

wherein t can be an integer from 0 to 5. t can be 1 to 5. tis 2 or 3. t can be 2. t can be 3.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

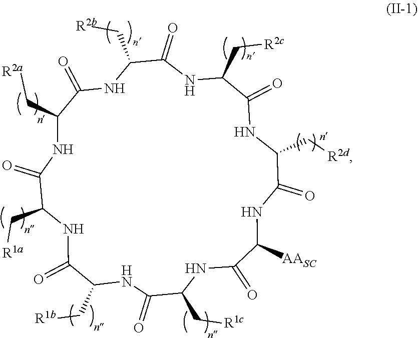

or protonated form thereof, wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

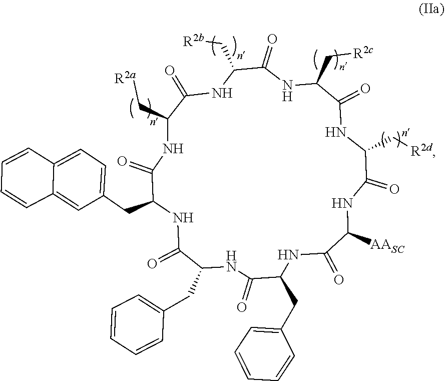

or protonated form thereof, wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

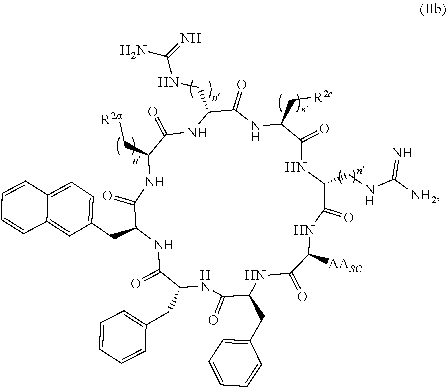

or protonated form thereof,
wherein R^{2a}, R^{2b}, AA_{SC}, and n′ are as defined herein.

The cCPP can have the structure of Formula (IIb):

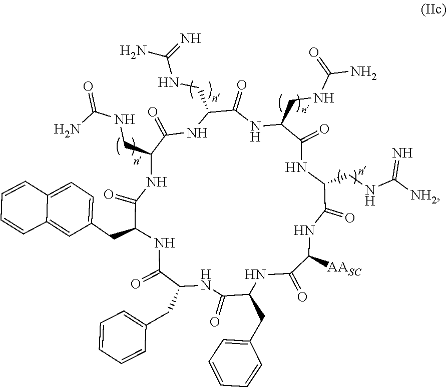

or a protonated form thereof, wherein:
AA_{SC} and n′ are as defined herein.

The cCPP of Formula (IIa) has one of the following structures:

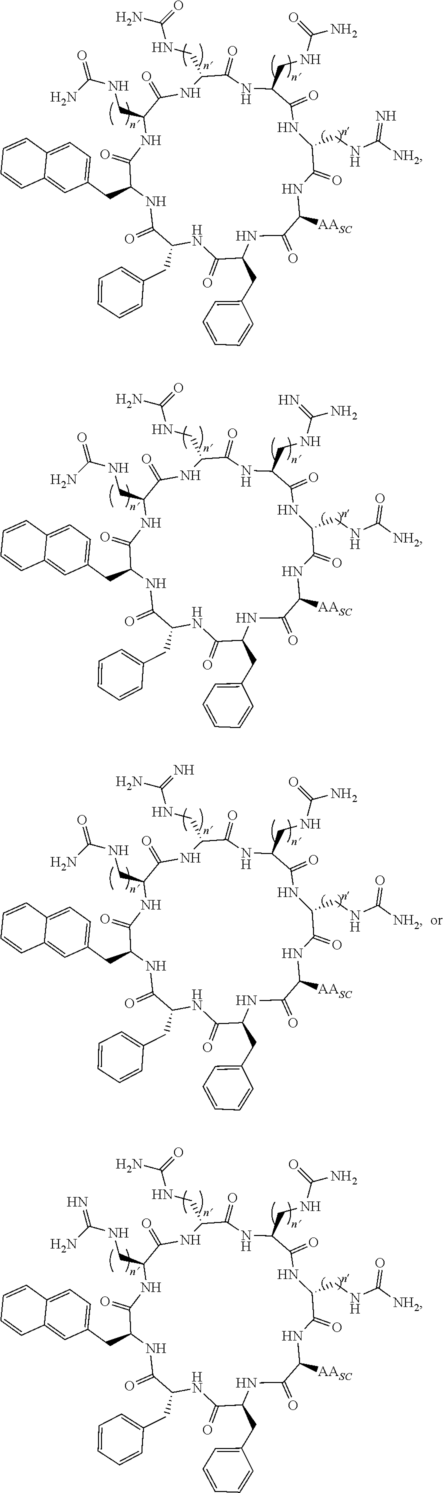

wherein AA_{SC} and n are as defined herein.

The cCPP of Formula (IIa) has one of the following structures:

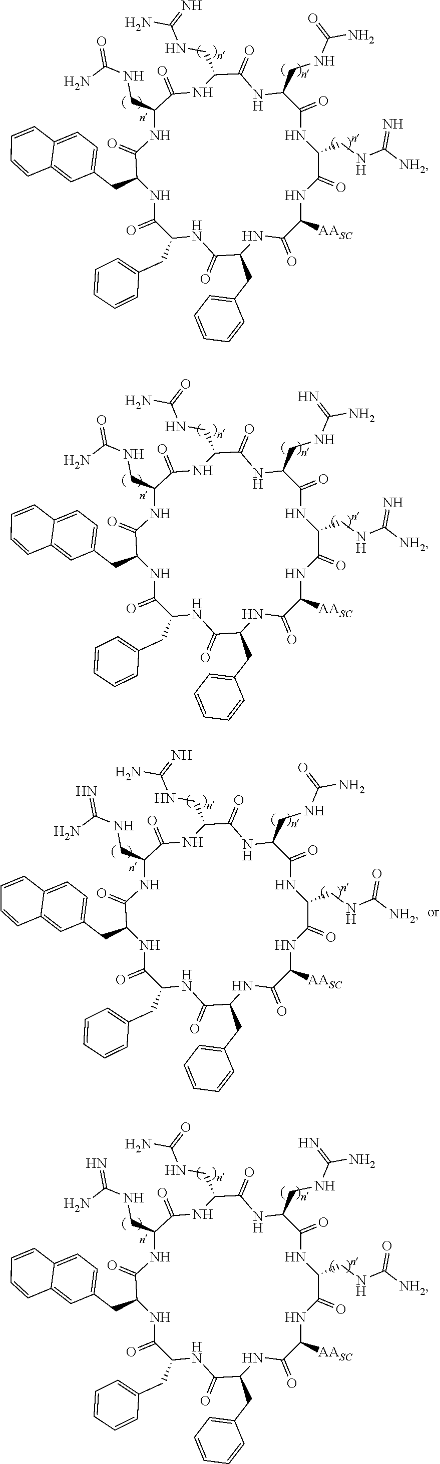

wherein AA_{SC} and n are as defined herein

The cCPP of Formula (IIa) has one of the following structures:

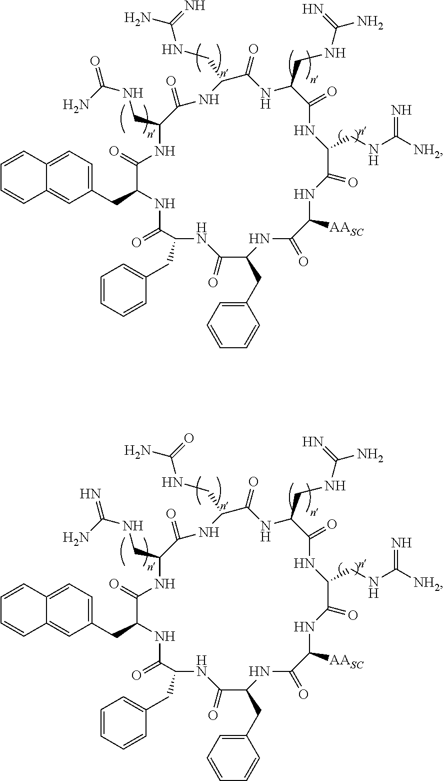

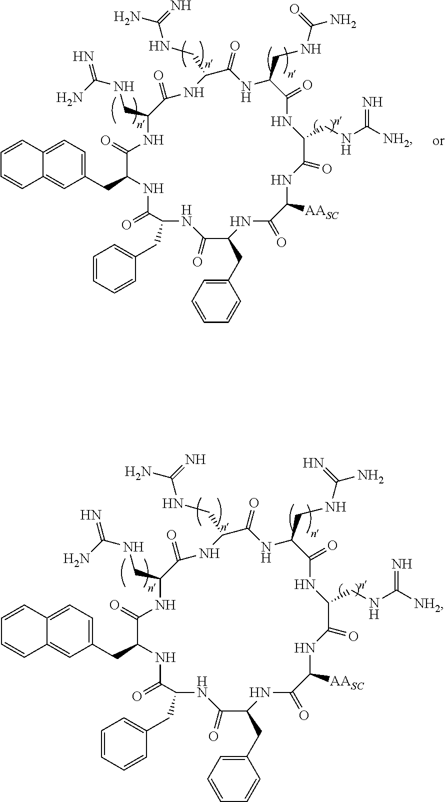

wherein AA_{SC} and n are as defined herein.

The cCPP of Formula (II) can have the structure:

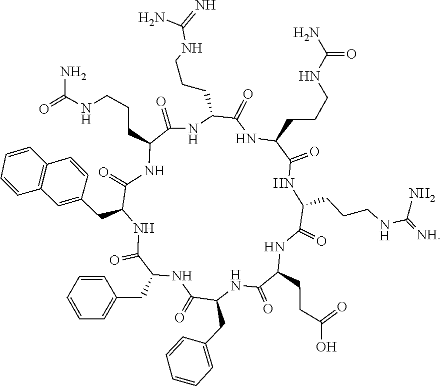 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

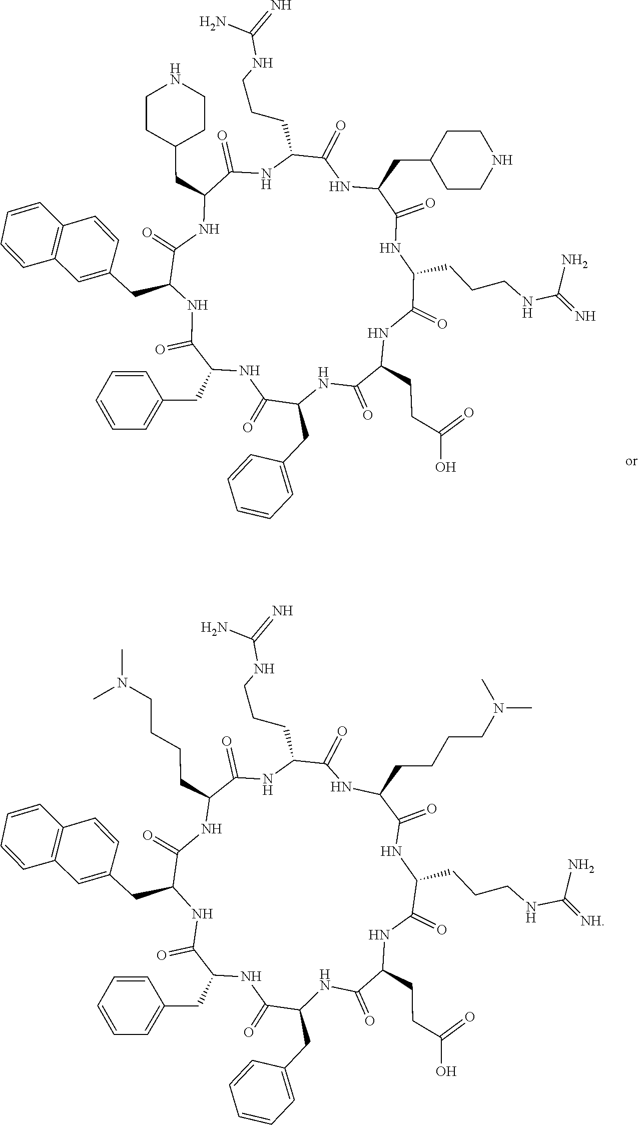 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O]

The cCPP can have the structure of Formula (III):

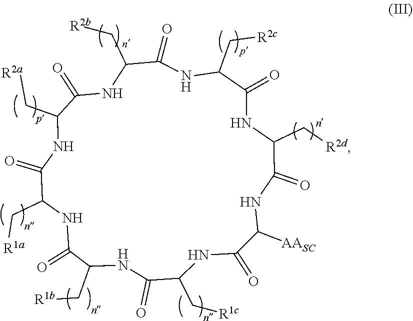

or protonated form thereof, wherein:
AA_{SC} is an amino acid side chain;
R^{1a}, R^{1b}, and R^{1c} are each independently a 6- to 14-membered aryl or a 6- to 14-membered heteroaryl;
R^{2a} and R_{2c} are each independently H,

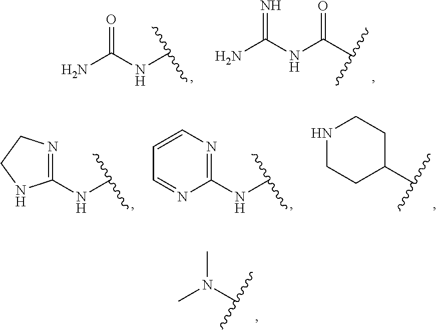

or a protonated form thereof;
R^{2b} and R^{2d} are each independently guanidine or a protonated form thereof;
each n″ is independently an integer from 1 to 3;
each n′ is independently an integer from 1 to 5; and
each p′ is independently an integer from 0 to 5.

The cCPP of Formula (III) can have the structure of Formula (III-1):

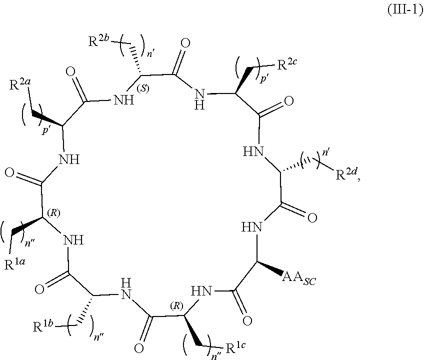

or protonated form thereof,
wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

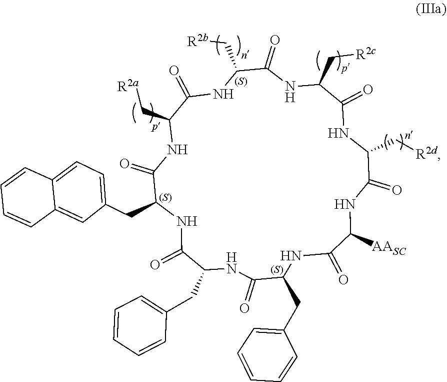

or protonated form thereof, wherein:
AA_{SC}, R^{2a}, R^{2c}, R^{2b}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R_{d} can each independently be guanidine or protonated form thereof, n″ can be 2 or 3, and each p′ can be 0.

p′ can 0. p′ can 1. p′ can 2. p′ can 3. p′ can 4. p′ can be 5.

The cCPP can have the structure:

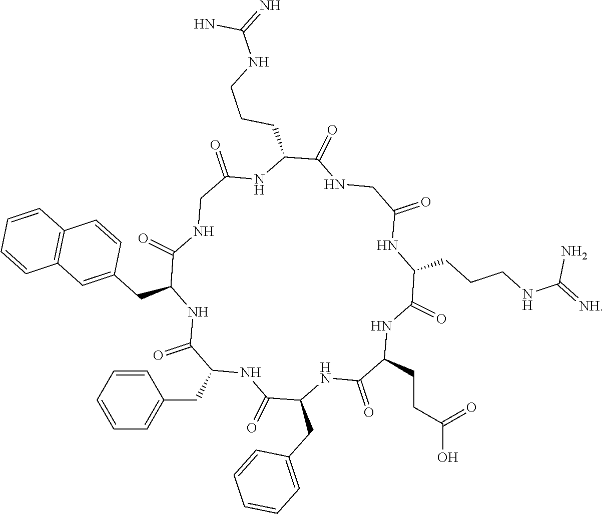 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

CPP Sequence

FfΦRrRrQ)
(FfΦCit-r-Cit-rQ)
(FfΦGrGrQ)
(FfFGRGRQ)
(FGFGRGRQ)
(GfFGrGrQ)
(FGFGRRRQ)
(FGFRRRRQ)

The cCPP of Formula (A) can be selected from:

CPP Sequence

FΦRRRRQ
fΦRrRrQ
FfΦRrRrQ
FfΦCit-r-Cit-rQ
FfΦGrGrQ
FfΦRGRGQ
FfFGRGRQ
FGFGRGRQ
GfFGrGrQ
FGFGRRRQ
FGFRRRRQ

In embodiments, the cCPP is selected from:

CPP sequence

| | | |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | FΦRrRrRQ |
| RRRΦFQ | RRΦFRRQ | FΦRRRRRQ |
| RRRRΦF | CRRRRFWQ | RRRRΦFDQC |
| FΦRRRR | FfΦRrRrQ | FΦRRR |
| FϕrRrRq | FFΦRRRRQ | FWRRR |
| FϕrRrRQ | RFRFRΦRQ | RRRΦF |
| FΦRRRRQ | URRRRFWQ | RRRWF |
| fΦRrRrQ | CRRRRFWQ | |

### Φ=L-naphthylalanine; ϕ=D-naphthylalanine; Ω=L-norleucine

AA_{SC} can be conjugated to a linker.

### Linker

The cCPP of the disclosure can be conjugated to a linker. The linker can link a cargo to the cCPP. The linker can be attached to the side chain of an amino acid of the cCPP, and the cargo can be attached at a suitable position on linker.

The linker can be any appropriate moiety which can conjugate a cCPP to one or more additional moieties, e.g., an exocyclic peptide (EP) and/or a cargo. Prior to conjugation to the cCPP and one or more additional moieties, the linker has two or more functional groups, each of which are independently capable of forming a covalent bond to the cCPP and one or more additional moieties. If the cargo is an oligonucleotide, the linker can be covalently bound to the 5′ end of the cargo or the 3′ end of the cargo. The linker can be covalently bound to the 5′ end of the cargo. The linker can be covalently bound to the 3′ end of the cargo. If the cargo is a peptide, the linker can be covalently bound to the N-terminus or the C-terminus of the cargo. The linker can be covalently bound to the backbone of the oligonucleotide or peptide cargo. The linker can be any appropriate moiety which conjugates a cCPP described herein to a cargo such as an oligonucleotide, peptide or small molecule.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more -(R¹-J-R²)z″-subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) -(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or -(R¹⁻J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z}′— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂)z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the cargo is conjugated to the linker.

The linker can comprises (i) a β alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) -(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

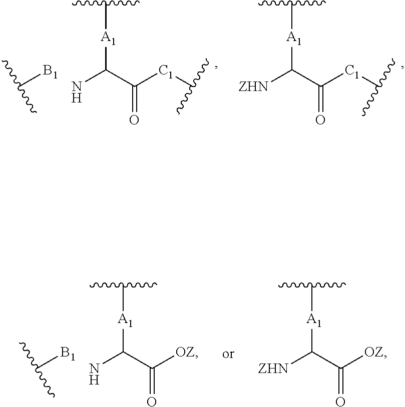

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ⁿ—COOH], or caspase-cleavage site (Val-Cit-PABC).

The hydrocarbon can be a residue of glycine or beta-alanine.

The linker can be bivalent and link the cCPP to a cargo. The linker can be bivalent and link the cCPP to an exocyclic peptide (EP).

The linker can be trivalent and link the cCPP to a cargo and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The linker can have the structure:

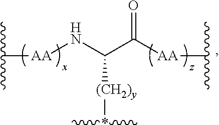

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the cargo through a linker (“L”). The linker can be conjugated to the cargo through a bonding group (“M”).

The linker can have the structure:

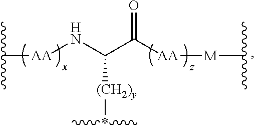

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

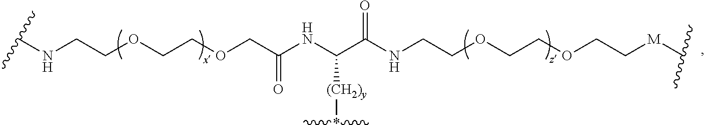

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

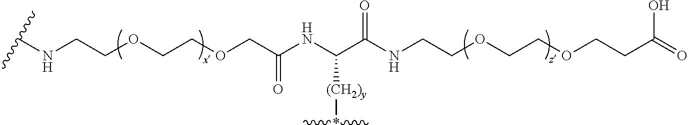

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to cargo at any suitable location on the cargo. The linker or M (wherein M is part of the linker) can be covalently bound to the 3′ end of oligonucleotide cargo or the 5′ end of an oligonucleotide cargo. The linker or M (wherein M is part of the linker) can be covalently bound to the N-terminus or the C-terminus of a peptide cargo. The linker or M (wherein M is part of the linker) can be covalently bound to the backbone of an oligonucleotide or a peptide cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can be bound to the side chain of aspartic acid, glutamic acid, glutamine, asparagine, or lysine, or a modified side chain of glutamine or asparagine (e.g., a reduced side chain having an amino group), on a peptide cargo. The linker can be bound to the side chain of lysine on the peptide cargo.

The linker can have a structure:

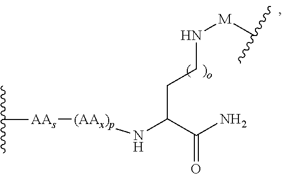

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

The linker can have a structure:

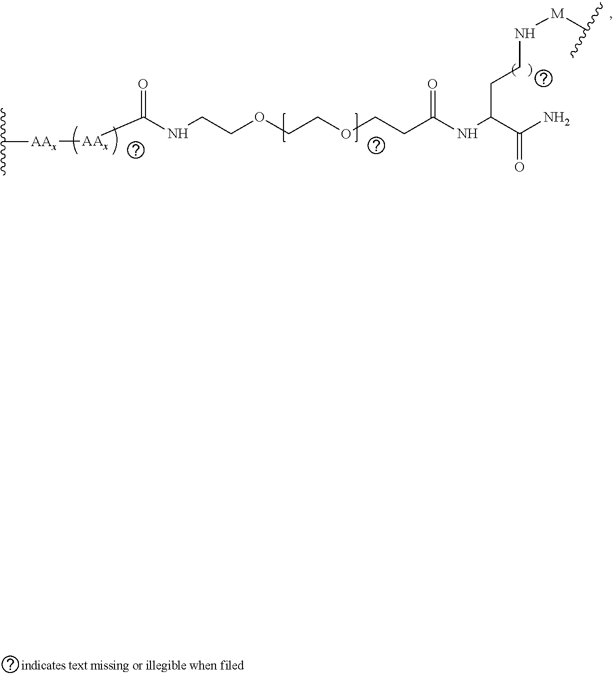

wherein
M is a group that conjugates L to a cargo, for example, an oligonucleotide;
AAₛ is a side chain or terminus of an amino acid on the cCPP;
each AAₓ is independently an amino acid residue;
is an integer from 0 to 10; and
p is an integer from 0 to 5.

M can comprise an alkylene, alkenylene, alkynylene, carbocyclyl, or heterocyclyl, each of which is optionally substituted. M can be selected from:

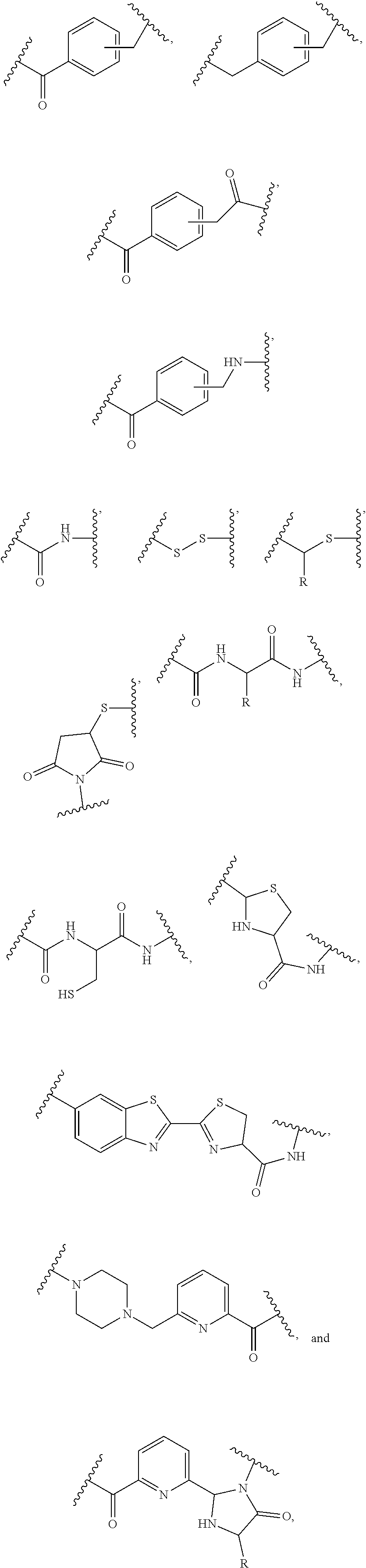

wherein R is alkyl, alkenyl, alkynyl, carbocyclyl, or heterocyclyl.

M can be selected from:

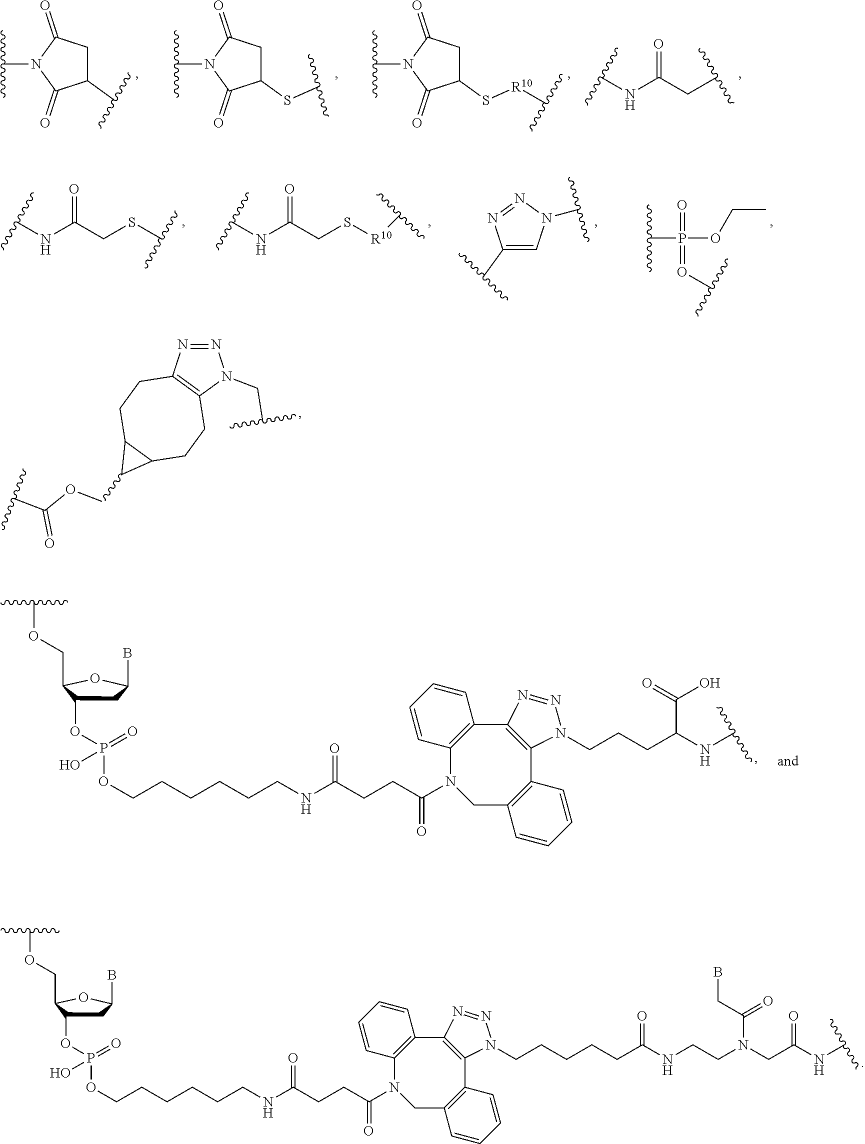

wherein: R¹⁰ is alkylene, cycloalkyl, or

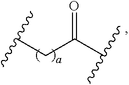

wherein a is 0 to 10.

M can be

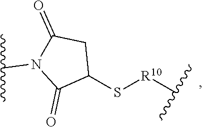

R¹⁰ can be

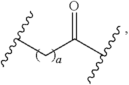

### and a is 0 to 10. M can be

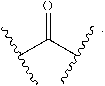 [SMILES: *C(*)=O]

M can be a heterobifunctional crosslinker, e.g.,

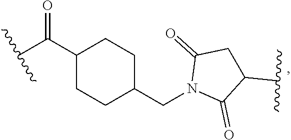 [SMILES: *C(=O)C1CCC(CN2C(=O)CC(*)C2=O)CC1]

which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

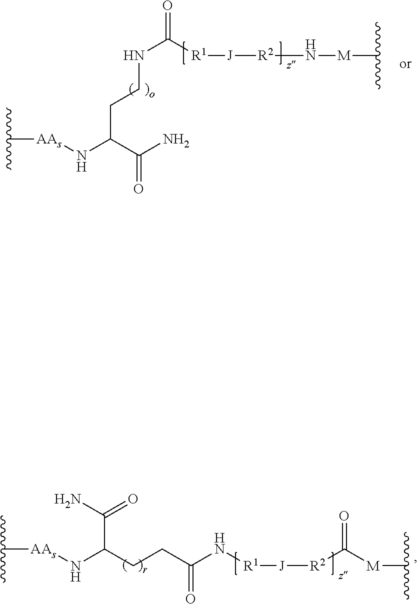

wherein M, AAₛ, each -(R¹-J-R²)z″-, o and z″ are defined herein; r can be 0 or 1.

r can be 0. r can be 1.

The linker can have the structure:

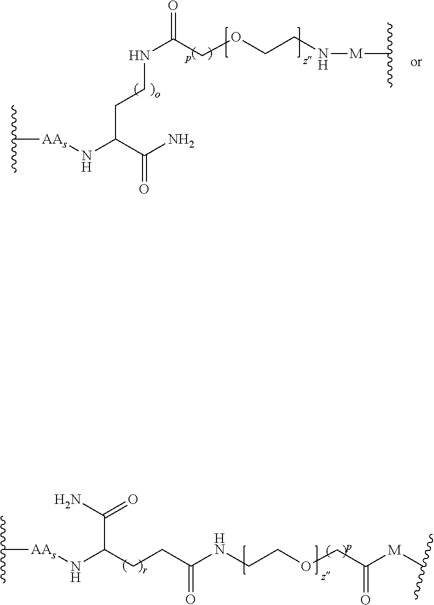

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

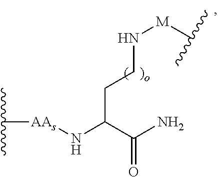

wherein:
M, AAₛ and o are as defined herein.

Other non-limiting examples of suitable linkers include:

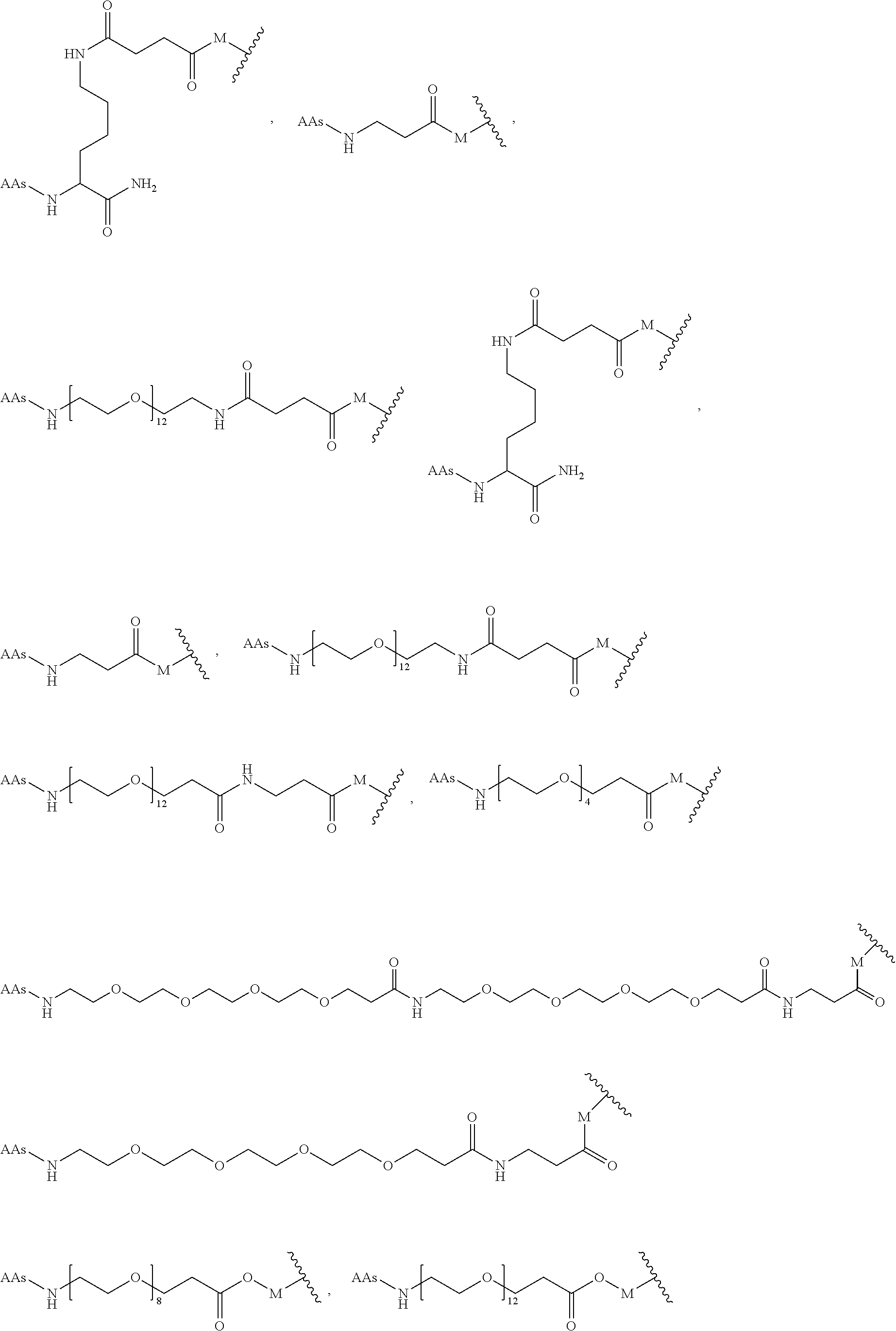

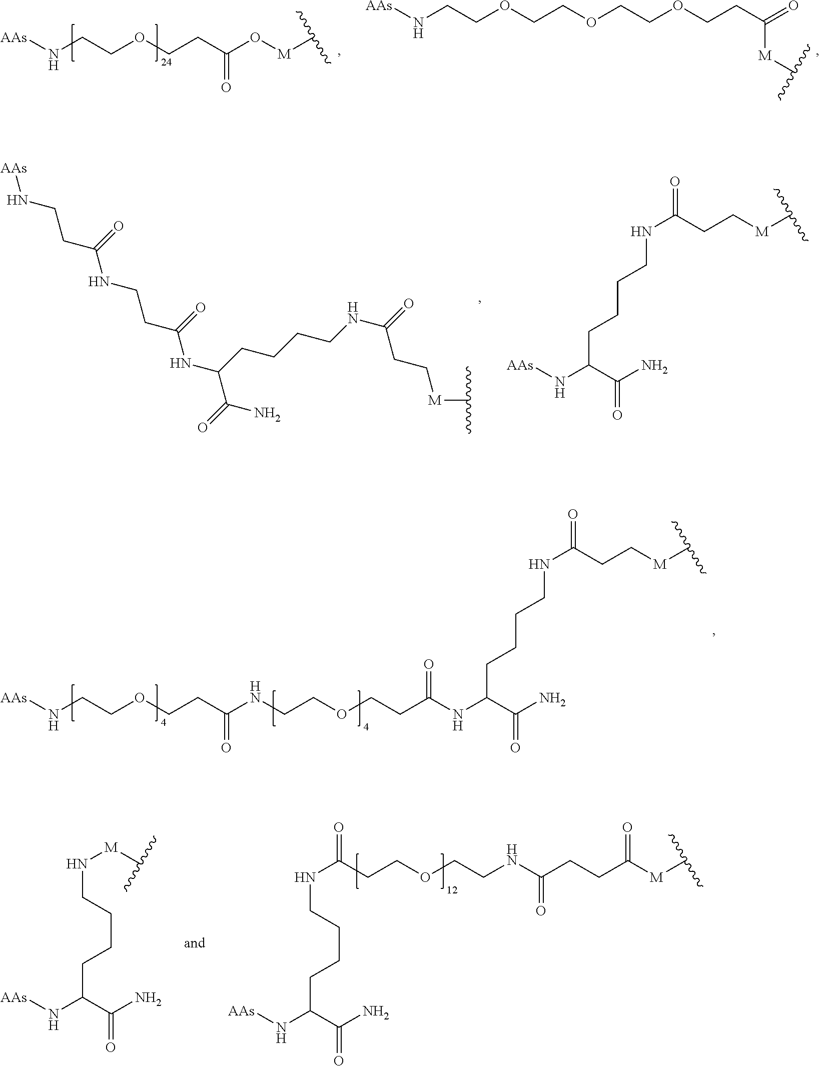

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

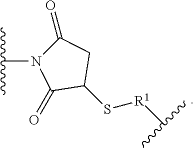

Provided herein is a compound comprising a cCPP and a cargo that comprises an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

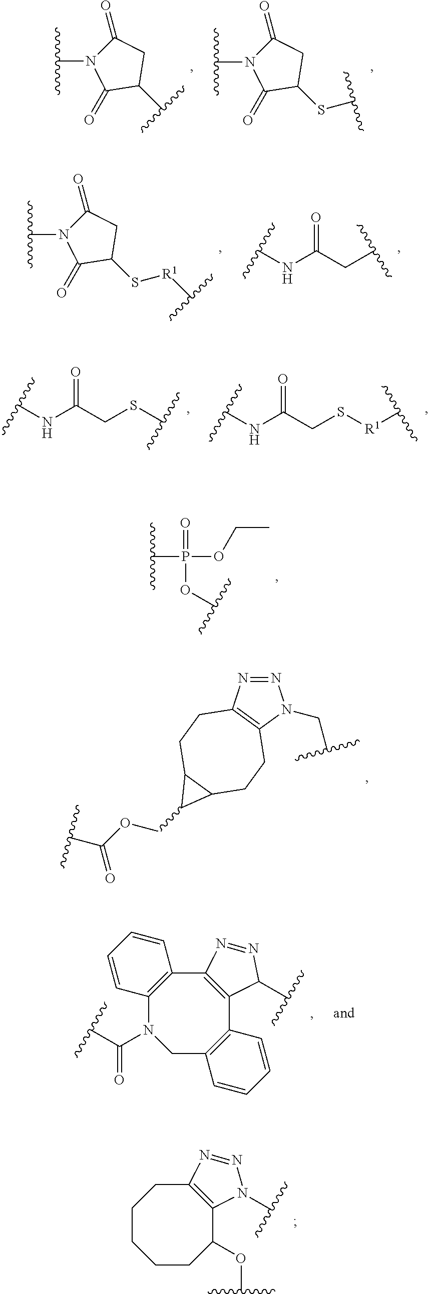

wherein: R¹ is alkylene, cycloalkyl, or

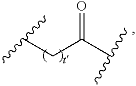

wherein t′ is 0 to 10 wherein each R is independently an alkyl, alkenyl,
alkynyl, carbocyclyl, or heterocyclyl, wherein R¹ is

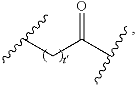

and t′ is 2.

The linker can have the structure:

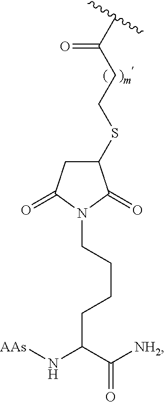

wherein AAₛ is as defined herein, and m′ is 0-10.

The linker can be of the formula:

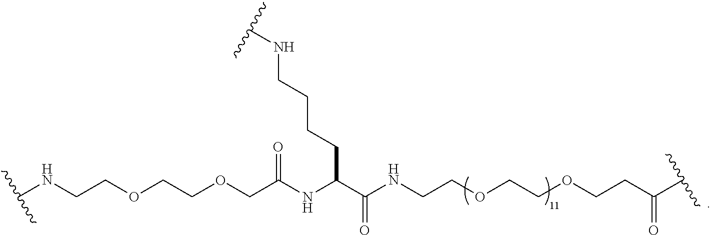

The linker can be of the formula:

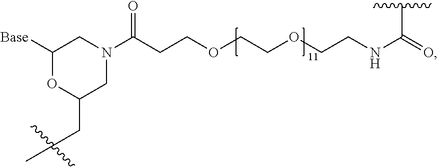

wherein “base” corresponds to a nucleobase at the 3′ end of a cargo phosphorodiamidate morpholino oligomer.

The linker can be of the formula:

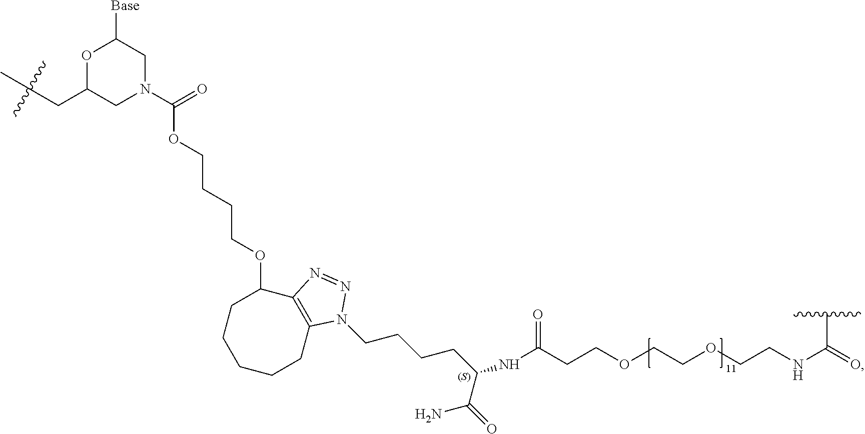

wherein “base” corresponds to a nucleobase at the 3′ end of a cargo phosphorodiamidate morpholino oligomer.

The linker can be of the formula:

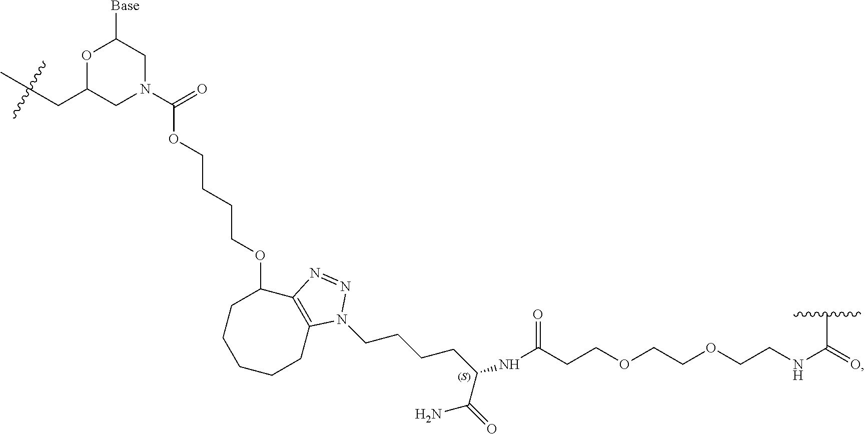

wherein “base” corresponds to a nucleobase at the 3′ end of a cargo phosphorodiamidate morpholino oligomer.

The linker can be of the formula:

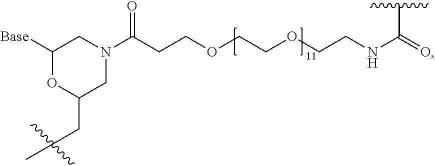

wherein “base” corresponds to a nucleobase at the 3′ end of a cargo phosphorodiamidate morpholino oligomer.

The linker can be of the formula:

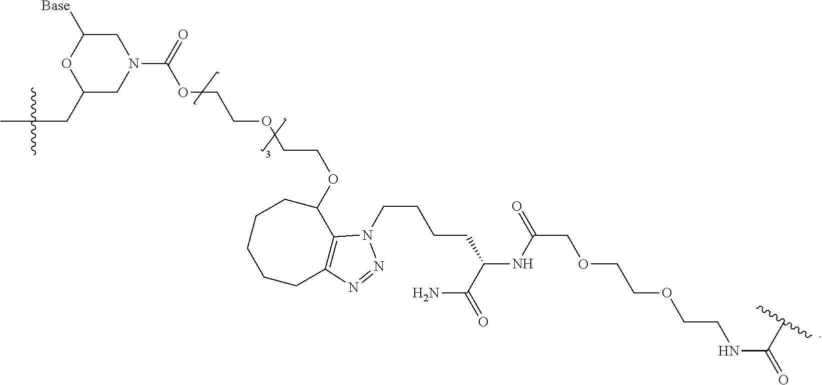

The linker an be covalently bound to a cargo at any suitable location on the cargo. The linker is covalently bound to the 3′ end of cargo or the 5′ end of an oligonucleotide cargo. The linker can be covalently bound to the backbone of a cargo.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 3:

TABLE 3
cCPP-linker conjugates

cyclo(FfΦ-4gp-r-4gp-rQ)-PEG₄-K-NH₂
cyclo(FfΦ-Cit-r-Cit-rQ)-PEG₄-K-NH₂
cyclo(FfΦ-Pia-r-Pia-rQ)-PEG₄-K-NH₂
cyclo(FfΦ-Dml-r-Dml-rQ)-PEG₄-K-NH₂
cyclo(FfΦ-Cit-r-Cit-rQ)-PEG₁₂-OH
cyclo(fΦR-Cit-R-Cit-Q)-PEG₁₂-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 4:

TABLE 4
cCPP-linker conjugate

Ac-PKKKRKV-Lys(cyclo[FfΦ-R-r-Cit-rQ])-PEG12-K(N₃)-
NH₂
Ac-PKKKRKV-Lys(cyclo[FfΦ-Cit-r-R-rQ])-PEG12-K(N₃)-
NH₂
Ac-PKKKRKV-K(cyclo(FfΦR-cit-R-cit-Q))-PEG12-
K(N₃)-NH₂
Ac-PKKKRKV-PEG2-Lys(cyclo[FfΦ-Cit-r-Cit-rQ])-B-
k(N₃)-NH₂
Ac-PKKKRKV-PEG2-Lys(cyclo[FfΦ-Cit-r-Cit-rQ])-PEG2-
k(N₃)-NH₂
Ac-PKKKRKV-PEG2-Lys(cyclo[FfΦ-Cit-r-Cit-rQ])-PEG4-
k(N₃)-NH₂
Ac-PKKKRKV-Lys(cyclo[FfΦ-Cit-r-Cit-rQ])-PEG12-
k(N₃)-NH₂
Ac-pkkkrkv-PEG2-Lys(cyclo[FfΦ-Cit-r-Cit-rQ])-
PEG12-k(N₃)-NH₂
Ac-rrv-PEG2-Lys(cyclo[FfΦ-Cit-r-Cit-rQ])-PEG12-OH
Ac-PKKKRKV-PEG2-Lys(cyclo[FfΦ-Cit-r-Cit-r-Q])-
PEG12-k(N₃)-NH₂
Ac-PKKK-Cit-KV-PEG2-Lys(cyclo[FfΦ-Cit-r-Cit-r-Q])-
PEG12-k(N₃)-NH₂
Ac-PKKKRKV-PEG2-Lys(cyclo[FfΦ-Cit-r-Cit-r-Q]-
PEG12-K(N₃)-NH₂

EEVs comprising a cyclic cell penetrating peptide (cCPP), linker and exocyclic peptide (EP) are provided. An EEV can comprise the structure of Formula (B):

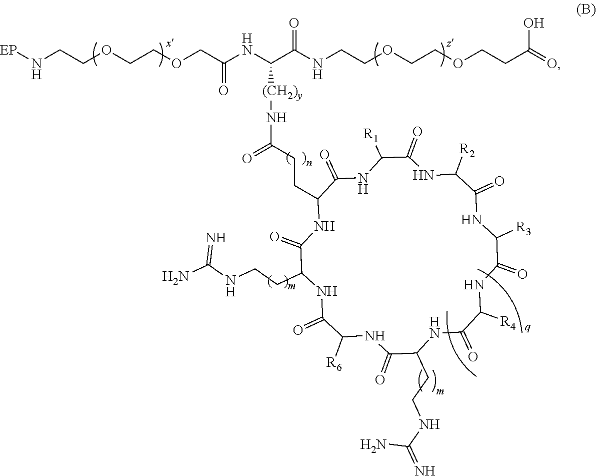

or a protonated form thereof,
wherein:
R₁, R₂, and R₃ are each independently H or an aromatic or heteroaromatic side chain of an amino acid;
R₄ and R₆ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.

R₁, R₂, R₃, R₄, R₇, EP, m, q, y, x′, z′ are as described herein.

n can be 0. n can be 1. n can be 2.

The EEV can comprise the structure of Formula (B-a) or (B-b):

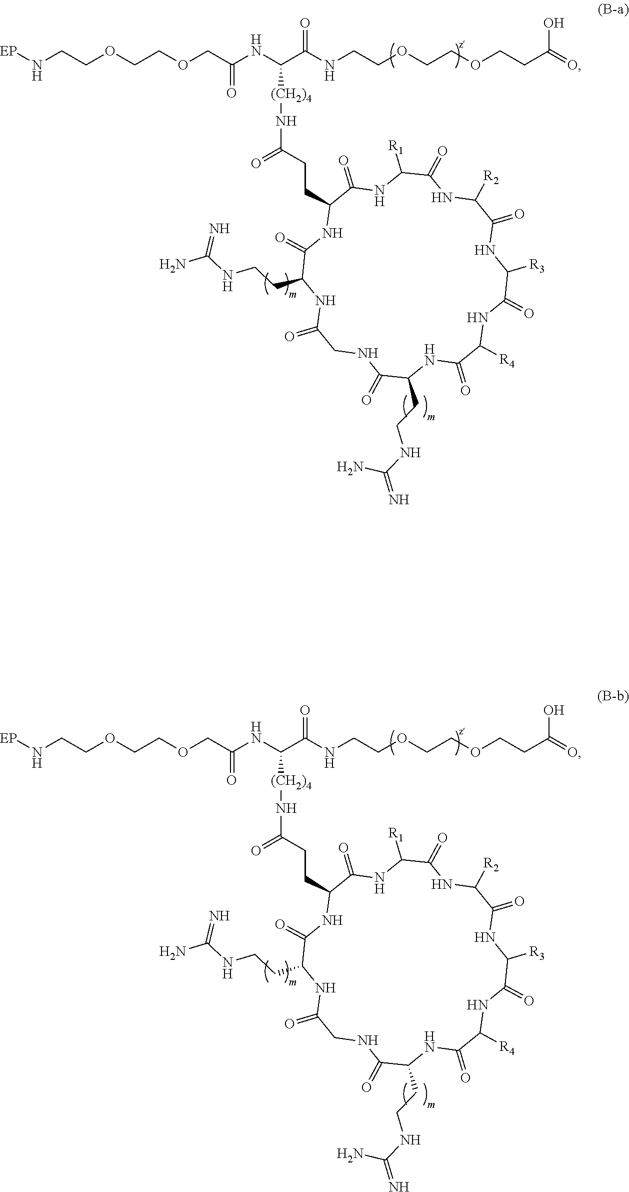

or a protonated form thereof, wherein EP, R¹, R², R³, R⁴, m and z′ are as defined above in Formula (B).

The EEV can comprises the structure of Formula (B-c):

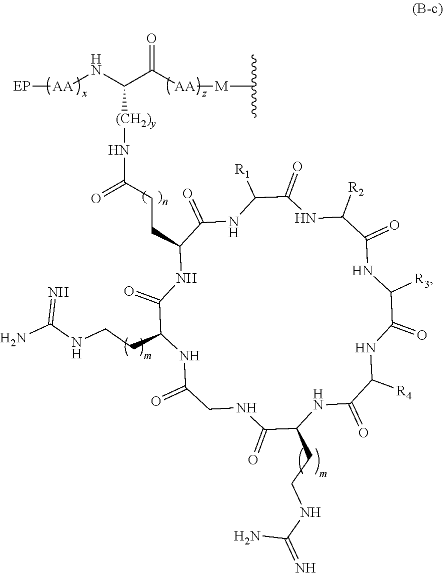

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

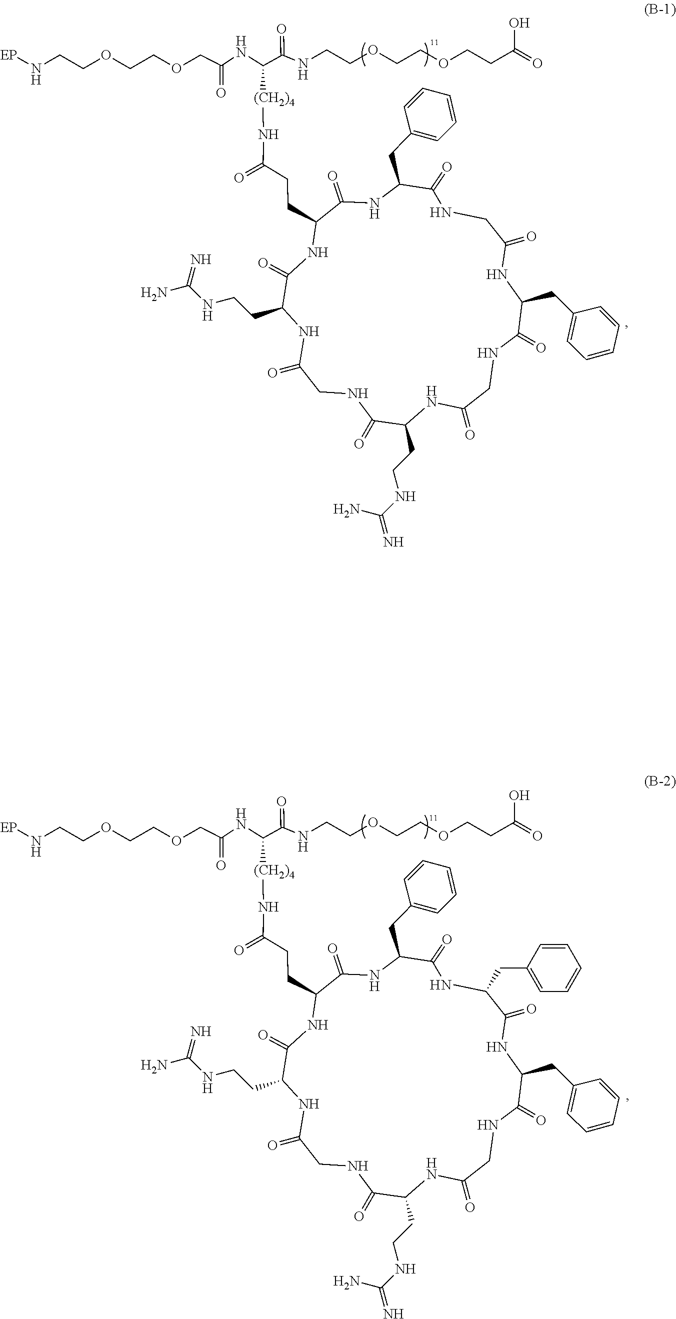

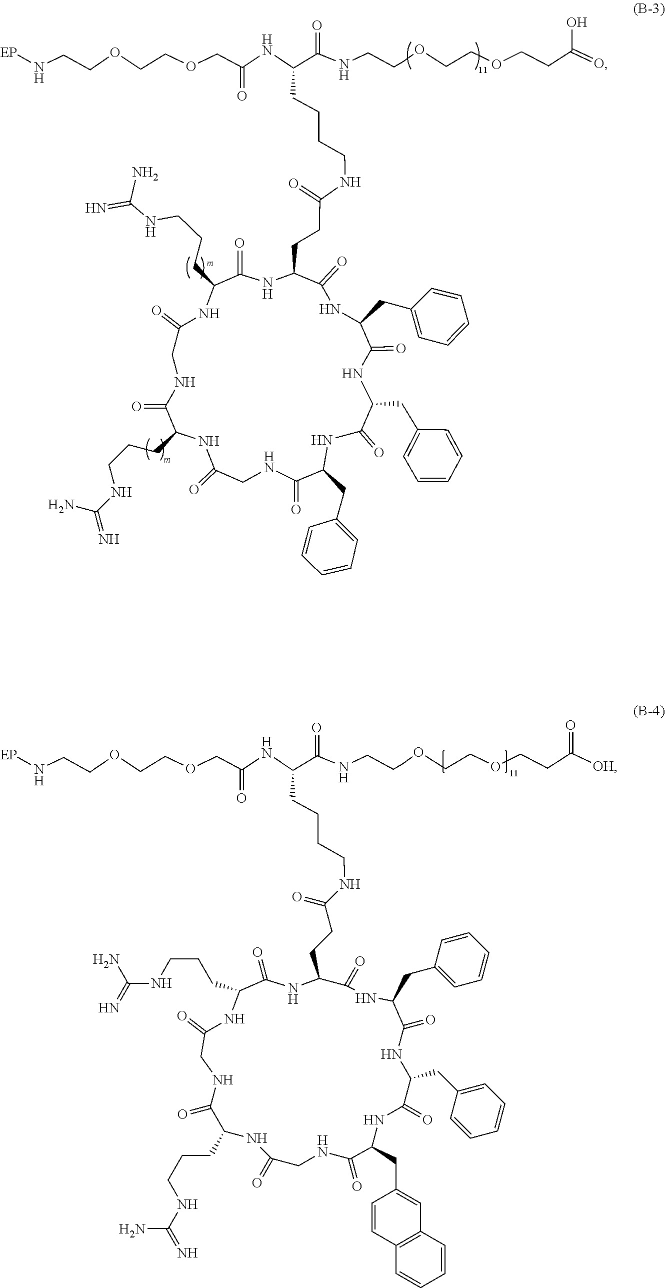

or a protonated form thereof, wherein EP is as defined above in Formula (B).

The EEV can comprise Formula (B) and can have the structure: Ac-PKKKRKVAEEA-K(cyclo[FGFGRGRQ])-PEG₁₂-OH or Ac-PKKKRKVAEEA-K(cyclo[GfFGrGrQ])-PEG₁₂-OH.

The EEV can comprise a cCPP of formula:

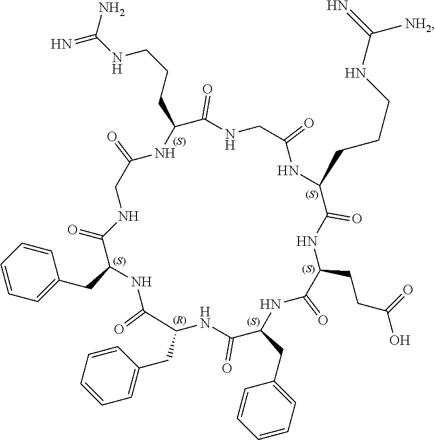

The EEV can comprise formula: Ac-PKKKRKV-miniPEG2-Lys(cyclo(FfFGRGRQ)-miniPEG2-K(N3).

The EEV can be Ac-P-K(Tfa)-K(Tfa)-K(Tfa)-R-K(Tfa)-V-AEEA-K-(cyclo[FGFGRGRQ])-PEG12-OH. The EEV can be:

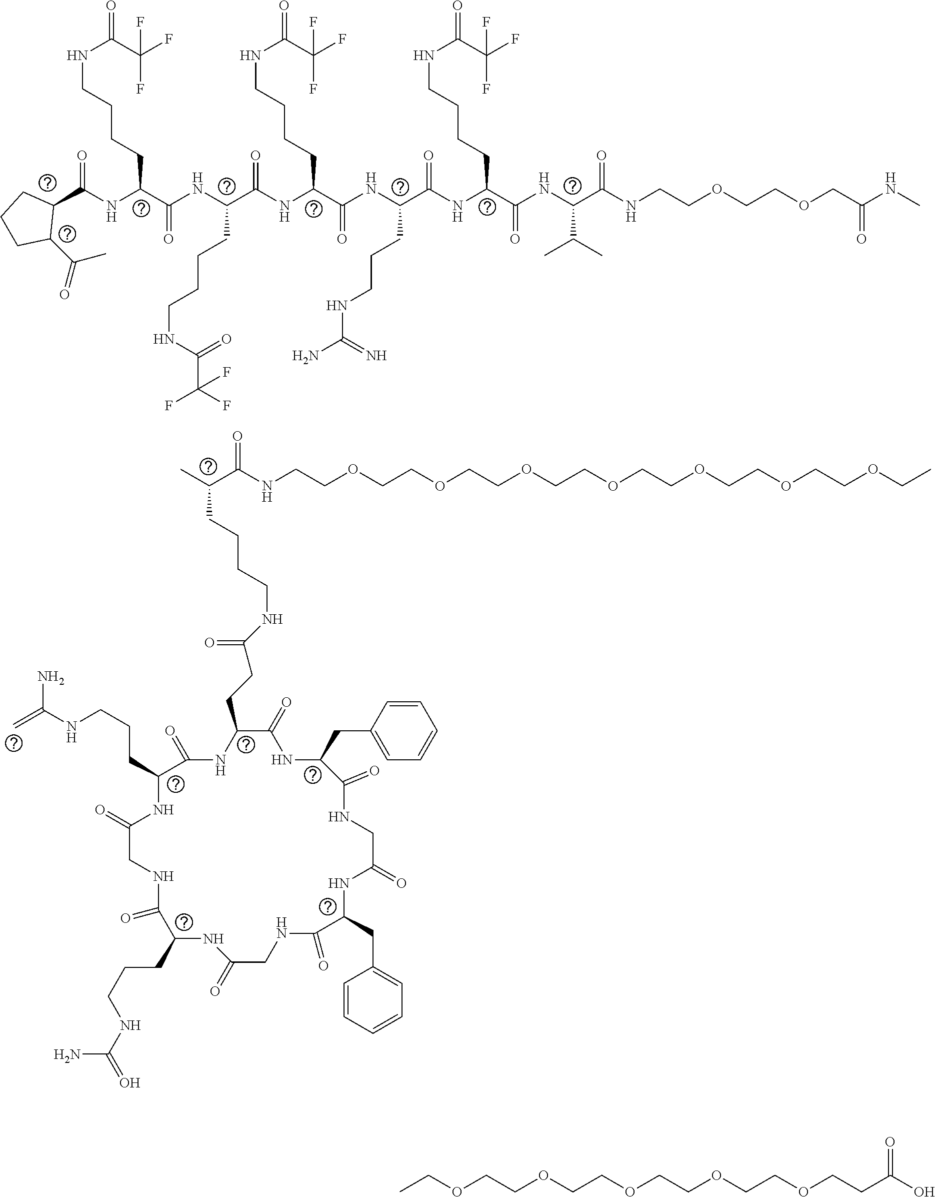

The EEV can be Ac-PKKKRKV-AEEA-Lys-(cyclo[FGFGRGRQ])-PEG12-OH. The EEV can be:

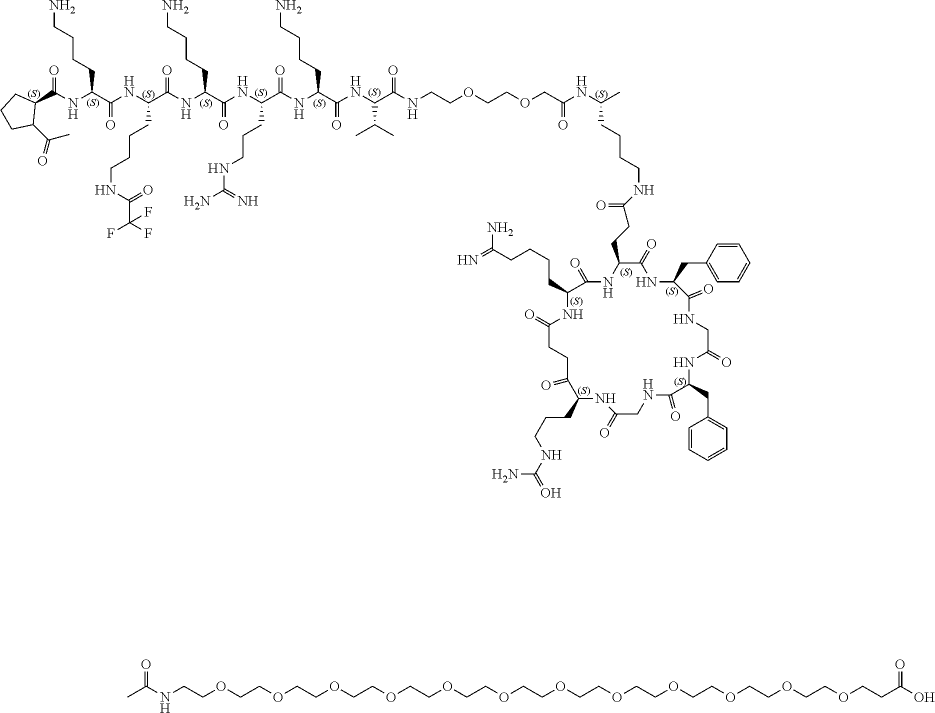

The EEV can be selected from
Ac-rr-miniPEG2-Dap[cyclo(FfΦ-Cit-r-Cit-rQ)]-PEG₁₂-OH
Ac-frr-PEG2-Dap(cyclo(FfΦ-Cit-r-Cit-rQ))-PEG₁₂-OH
Ac-rfr-PEG2-Dap(cyclo(FfΦ-Cit-r-Cit-rQ))-PEG₁₂-OH
Ac-rbfbr-PEG2-Dap(cyclo(FfΦ-Cit-r-Cit-rQ))-PEG₁₂-OH
Ac-rrr-PEG2-Dap(cyclo(FfΦ-Cit-r-Cit-rQ))-PEG₁₂-OH
Ac-rbr-PEG2-Dap(cyclo(FfΦ-Cit-r-Cit-rQ))-PEG₁₂-OH
Ac-rbrbr-PEG2-Dap(cyclo(FfΦ-Cit-r-Cit-rQ))-PEG₁₂-OH
Ac-hh-PEG2-Dap(cyclo(FfΦ-Cit-r-Cit-rQ))-PEG₁₂-OH
Ac-hbh-PEG2-Dap(cyclo(FfΦ-Cit-r-Cit-rQ))-PEG₁₂-OH
Ac-hbhbh-PEG2-Dap(cyclo(FfΦ-Cit-r-Cit-rQ))-PEG₁₂-OH
Ac-rbhbh-PEG2-Dap(cyclo(FfΦ-Cit-r-Cit-rQ))-PEG₁₂-OH
Ac-hbrbh-PEG2-Dap(cyclo(FfΦ-Cit-r-Cit-rQ))-PEG₁₂-OH
Ac-rr-Dap(cyclo(FfΦ-Cit-r-Cit-rQ))-b-OH
Ac-frr-Dap(cyclo(FfΦ-Cit-r-Cit-rQ))-b-OH
Ac-rfr-Dap(cyclo(FfΦ-Cit-r-Cit-rQ))-b-OH
Ac-rbfbr-Dap(cyclo(FfΦ-Cit-r-Cit-rQ))-b-OH
Ac-rrr-Dap(cyclo(FfΦ-Cit-r-Cit-rQ))-b-OH
Ac-rbr-Dap(cyclo(FfΦ-Cit-r-Cit-rQ))-b-OH
Ac-rbrbr-Dap(cyclo(FfΦ-Cit-r-Cit-rQ))-b-OH
Ac-hh-Dap(cyclo(FfΦ-Cit-r-Cit-rQ))-b-OH
Ac-hbh-Dap(cyclo(FfΦ-Cit-r-Cit-rQ))-b-OH
Ac-hbhbh-Dap(cyclo(FfΦ-Cit-r-Cit-rQ))-b-OH
Ac-rbhbh-Dap(cyclo(FfΦ-Cit-r-Cit-rQ))-b-OH
Ac-hbrbh-Dap(cyclo(FfΦ-Cit-r-Cit-rQ))-b-OH
Ac-KKKK-miniPEG₂-Lys(cyclo(Ff-Nal-GrGrQ))-miniPEG₂-K(N₃)-NH₂
Ac-KGKK-miniPEG₂-Lys(cyclo(Ff-Nal-GrGrQ))-miniPEG₂-K(N₃)-NH₂
Ac-KKGK-miniPEG₂-Lys(cyclo(Ff-Nal-GrGrQ))-miniPEG₂-K(N₃)-NH₂
Ac-KKK-miniPEG₂-Lys(cyclo(Ff-Nal-GrGrQ))-miniPEG₂-K(N₃)-NH₂
Ac-KK-miniPEG₂-Lys(cyclo(Ff-Nal-GrGrQ))-miniPEG₂-K(N₃)-NH₂
Ac-KGK-miniPEG₂-Lys(cyclo(Ff-Nal-GrGrQ))-miniPEG₂-K(N₃)-NH₂
Ac-KBK-miniPEG₂-Lys(cyclo(Ff-Nal-GrGrQ))-miniPEG₂-K(N₃)-NH₂
Ac-KBKBK-miniPEG₂-Lys(cyclo(Ff-Nal-GrGrQ))-miniPEG₂-K(N₃)-NH₂
Ac-KR-miniPEG₂-Lys(cyclo(Ff-Nal-GrGrQ))-miniPEG₂-K(N₃)-NH₂
Ac-KBR-miniPEG₂-Lys(cyclo(Ff-Nal-GrGrQ))-miniPEG₂-K(N₃)-NH₂
Ac-PKKKRKV-miniPEG₂-Lys(cyclo(Ff-Nal-GrGrQ))-miniPEG₂-K(N₃)-NH₂
Ac-PKKKRKV-miniPEG₂-Lys(cyclo(Ff-Nal-GrGrQ))-miniPEG₂-K(N₃)-NH₂
Ac-PGKKRKV-miniPEG₂-Lys(cyclo(Ff-Nal-GrGrQ))-miniPEG₂-K(N₃)-NH₂
Ac-PKGKRKV-miniPEG₂-Lys(cyclo(Ff-Nal-GrGrQ))-miniPEG₂-K(N₃)-NH₂
Ac-PKKGRKV-miniPEG₂-Lys(cyclo(Ff-Nal-GrGrQ))-miniPEG₂-K(N₃)-NH₂
Ac-PKKKGKV-miniPEG₂-Lys(cyclo(Ff-Nal-GrGrQ))-miniPEG₂-K(N₃)-NH₂
Ac-PKKKRGV-miniPEG₂-Lys(cyclo(Ff-Nal-GrGrQ))-miniPEG₂-K(N₃)-NH₂
Ac-PKKKRKG-miniPEG₂-Lys(cyclo(Ff-Nal-GrGrQ))-miniPEG₂-K(N₃)-NH₂
Ac-KKKRK-miniPEG₂-Lys(cyclo(Ff-Nal-GrGrQ))-miniPEG₂-K(N₃)-NH₂
Ac-KKRK-miniPEG₂-Lys(cyclo(Ff-Nal-GrGrQ))-miniPEG₂-K(N₃)-NH₂ and
Ac-KRK-miniPEG₂-Lys(cyclo(Ff-Nal-GrGrQ))-miniPEG₂-K(N₃)-NH₂.

The EEV can be selected from:
Ac-PKKKRKV-Lys(cyclo[FfΦGrGrQ])-PEG₁₂-K(N₃)-NH₂
Ac-PKKKRKV-miniPEG₂-Lys(cyclo[FfΦGrGrQ])-miniPEG₂-K(N₃)-NH₂
Ac-PKKKRKV-miniPEG₂-Lys(cyclo[FGFGRGRQ])-miniPEG₂-K(N₃)-NH₂
Ac-KR-PEG₂-K(cyclo[FGFGRGRQ])-PEG₂-K(N₃)-NH₂
Ac-PKKKGKV-PEG₂-K(cyclo[FGFGRGRQ])-PEG₂-K(N₃)-NH₂
Ac-PKKKRKG-PEG₂-K(cyclo[FGFGRGRQ])-PEG₂-K(N₃)-NH₂
Ac-KKKRK-PEG₂-K(cyclo[FGFGRGRQ])-PEG₂-K(N₃)-NH₂
Ac-PKKKRKV-miniPEG₂-Lys(cyclo[FFΦGRGRQ])-miniPEG₂-K(N₃)-NH₂
Ac-PKKKRKV-miniPEG₂-Lys(cyclo[βhFfΦGrGrQ])-miniPEG₂-K(N₃)-NH₂ and
Ac-PKKKRKV-miniPEG₂-Lys(cyclo[FfΦSrSrQ])-miniPEG₂-K(N₃)-NH₂.

The EEV can be selected from:
Ac-PKKKRKV-miniPEG₂-Lys(cyclo(GfFGrGrQ])-PEG₁₂-OH
Ac-PKKKRKV-miniPEG₂-Lys(cyclo[FGFKRKRQ])-PEG₁₂-OH
Ac-PKKKRKV-miniPEG₂-Lys(cyclo[FGFRGRGQ])-PEG₁₂-OH
Ac-PKKKRKV-miniPEG₂-Lys(cyclo[FGFGRGRGRQ])-PEG₁₂-OH
Ac-PKKKRKV-miniPEG₂-Lys(cyclo[FGFGRrRQ])-PEG₁₂-OH
Ac-PKKKRKV-miniPEG₂-Lys(cyclo[FGFGRRRQ])-PEG₁₂-OH and
Ac-PKKKRKV-miniPEG₂-Lys(cyclo[FGFRRRRQ])-PEG₁₂-OH.

The EEV can be selected from:
Ac-KKKRKG-miniPEG₂-K(cyclo[FGFGRGRQ])-PEG₁₂-OH
Ac-KKKRK-miniPEG₂-K(cyclo[FGFGRGRQ])-PEG₁₂-OH
Ac-KKRKK-PEG₄-K(cyclo[FGFGRGRQ])-PEG₁₂-OH
Ac-KRKKK-PEG₄-K(cyclo[FGFGRGRQ])-PEG₁₂-OH
Ac-KKKKR-PEG₄-K(cyclo[FGFGRGRQ])-PEG₁₂-OH
Ac-RKKKK-PEG₄-K(cyclo[FGFGRGRQ])-PEG₁₂-OH and
Ac-KKKRK-PEG₄-K(cyclo[FGFGRGRQ])-PEG₁₂-OH.

The EEV can be selected from:
Ac-PKKKRKV-PEG₂-K(cyclo[FGFGRGRQ])-PEG₂-K(N₃)-NH₂
Ac-PKKKRKV-PEG₂-K(cyclo[FGFGRGRQ])-PEG₁₂-OH
Ac-PKKKRKV-PEG₂-K(cyclo[GfFGrGrQ])-PEG₂-K(N₃)-NH₂ and
Ac-PKKKRKV-PEG₂-K(cyclo[GfFGrGrQ])-PEG₁₂-OH.

The cargo can be an AC and the EEV can be selected from:
Ac-PKKKRKV-PEG₂-K(cyclo[FfΦGrGrQ])-PEG₁₂-OH
Ac-PKKKRKV-PEG₂-K(cyclo[FfΦCit-r-Cit-rQ])-PEG₁₂-OH
Ac-PKKKRKV-PEG₂-K(cyclo[FfFGRGRQ])-PEG₁₂-OH
Ac-PKKKRKV-PEG₂-K(cyclo[FGFGRGRQ])-PEG₁₂-OH
Ac-PKKKRKV-PEG₂-K(cyclo[GfFGrGrQ])-PEG₁₂-OH
Ac-PKKKRKV-PEG₂-K(cyclo[FGFGRRRQ])-PEG₁₂-OH
Ac-PKKKRKV-PEG₂-K(cyclo[FGFRRRRQ])-PEG₁₂-OH
Ac-rr-PEG₂-K(cyclo[FfΦGrGrQ])-PEG₁₂-OH
Ac-rr-PEG₂-K(cyclo[FfΦCit-r-Cit-rQ])-PEG₁₂-OH
Ac-rr-PEG₂-K(cyclo[FfF-GRGRQ])-PEG₁₂-OH
Ac-rr-PEG₂-K(cyclo[FGFGRGRQ])-PEG₁₂-OH
Ac-rr-PEG₂-K(cyclo[GfFGrGrQ])-PEG₁₂-OH
Ac-rr-PEG₂-K(cyclo[FGFGRRRQ])-PEG₁₂-OH
Ac-rr-PEG₂-K(cyclo[FGFRRRRQ])-PEG₁₂-OH
Ac-rrr-PEG₂-K(cyclo[FfΦGrGrQ])-PEG₁₂-OH
Ac-rrr-PEG₂-K(cyclo[FfΦCit-r-Cit-rQ])-PEG₁₂-OH
Ac-rrr-PEG₂-K(cyclo[FfFGRGRQ])-PEG₁₂-OH
Ac-rrr-PEG₂-K(cyclo[FGFGRGRQ])-PEG₁₂-OH
Ac-rrr-PEG₂-K(cyclo[GfFGrGrQ])-PEG₁₂-OH
Ac-rrr-PEG₂-K(cyclo[FGFGRRRQ])-PEG₁₂-OH
Ac-rrr-PEG₂-K(cyclo[FGFRRRRQ])-PEG₁₂-OH
Ac-rhr-PEG₂-K(cyclo[FfΦGrGrQ])-PEG₁₂-OH
Ac-rhr-PEG₂-K(cyclo[FfΦCit-r-Cit-rQ])-PEG₁₂-OH
Ac-rhr-PEG₂-K(cyclo[FfFGRGRQ])-PEG₁₂-OH
Ac-rhr-PEG₂-K(cyclo[FGFGRGRQ])-PEG₁₂-OH
Ac-rhr-PEG₂-K(cyclo[GfFGrGrQ])-PEG₁₂-OH
Ac-rhr-PEG2-K(cyclo[FGFGRRRQ])-PEG12-OH
Ac-rhr-PEG2-K(cyclo[FGFRRRRQ])-PEG12-OH
Ac-rbr-PEG2-K(cyclo[FfΦGrGrQ])-PEG12-OH
Ac-rbr-PEG₂-K(cyclo[FfΦCit-r-Cit-rQ])-PEG₁₂-OH
Ac-rbr-PEG₂-K(cyclo[FfFGRGRQ])-PEG₁₂-OH
Ac-rbr-PEG₂-K(cyclo[FGFGRGRQ])-PEG₁₂-OH
Ac-rbr-PEG₂-K(cyclo[GfFGrGrQ])-PEG₁₂-OH
Ac-rbr-PEG₂-K(cyclo[FGFGRRRQ])-PEG₁₂-OH
Ac-rbr-PEG₂-K(cyclo[FGFRRRRQ])-PEG₁₂-OH
Ac-rbrbr-PEG₂-K(cyclo[FfΦGrGrQ])-PEG₁₂-OH
Ac-rbrbr-PEG₂-K(cyclo[FfΦCit-r-Cit-rQ])-PEG₁₂-OH
Ac-rbrbr-PEG₂-K(cyclo[FfFGRGRQ])-PEG₁₂-OH
Ac-rbrbr-PEG₂-K(cyclo[FGFGRGRQ])-PEG₁₂-OH
Ac-rbrbr-PEG₂-K(cyclo[GfFGrGrQ])-PEG₁₂-OH
Ac-rbrbr-PEG₂-K(cyclo[FGFGRRRQ])-PEG₁₂-OH
Ac-rbrbr-PEG₂-K(cyclo[FGFRRRRQ])-PEG₁₂-OH
Ac-rbhbr-PEG₂-K(cyclo[FfΦGrGrQ])-PEG₁₂-OH
Ac-rbhbr-PEG₂-K(cyclo[FfΦCit-r-Cit-rQ])-PEG₁₂-OH
Ac-rbhbr-PEG₂-K(cyclo[FfFGRGRQ])-PEG₁₂-OH
Ac-rbhbr-PEG₂-K(cyclo[FGFGRGRQ])-PEG₁₂-OH
Ac-rbhbr-PEG₂-K(cyclo[GfFGrGrQ])-PEG₁₂-OH
Ac-rbhbr-PEG₂-K(cyclo[FGFGRRRQ])-PEG₁₂-OH
Ac-rbhbr-PEG₂-K(cyclo[FGFRRRRQ])-PEG₁₂-OH
Ac-hbrbh-PEG₂-K(cyclo[FfΦGrGrQ])-PEG₁₂-OH
Ac-hbrbh-PEG₂-K(cyclo[FfΦCit-r-Cit-rQ])-PEG₁₂-OH
Ac-hbrbh-PEG₂-K(cyclo[FfFGRGRQ])-PEG₁₂-OH
Ac-hbrbh-PEG₂-K(cyclo[FGFGRGRQ])-PEG₁₂-OH
Ac-hbrbh-PEG₂-K(cyclo[GfFGrGrQ])-PEG₁₂-OH
Ac-hbrbh-PEG₂-K(cyclo[FGFGRRRQ])-PEG₁₂-OH and
Ac-hbrbh-PEG₂-K(cyclo[FGFRRRRQ])-PEG₁₂-OH,
   wherein b is beta-alanine, and the exocyclic sequence can be D or L stereochemistry.

In embodiments, the cCPP can be

Structure

| | | |
|---|---|---|
| cyclo(fΦRrRQ) | cyclo(FΦFRRRQ) | cyclo(hF-fΦRrRrQ) |
| cyclo(fΦrRrQ) | cyclo(fFfrRrQ) | cyclo(Sta-fΦRrRrQ) |
| cyclo(FΦrRrQ) | cyclo(fFfRrRQ) | cyclo(Tba-fΦRrRrQ) |
| cyclo(FΦRrRQ) | cyclo(fFϕrRrQ) | cyclo(Ff-1Nal-RrRrQ) |
| cyclo(FϕRrRQ) | cyclo(fΦfrRrQ) | cyclo(Ff-Bpa-RrRrQ) |
| cyclo(FϕrRrQ) | cyclo(ϕFfrRrQ) | cyclo(Ff-3aa-RrRrQ) |
| cyclo(f-G[decyl]rRrQ) | cyclo(FFFRRRRQ) | cyclo(Ff-1aa-RrRrQ) |
| cyclo(f-G[dodecyl]rRrQ) | cyclo(RFRFRFRQ) | cyclo(FfΦ-Agp-r-Agp-rQ) |
| cyclo(f-D[dodecenamide]rRrQ) | cyclo(FfFRrRrQ) | cyclo(FfΦ-Agb-r-Agb-rQ) |
| cyclo(f-D[tetradecanamide]rRrQ) | cyclo(fFfrRrRQ) | cyclo(FfΦ-hR-r-hR-rQ) |
| cyclo(f-D[hexadecanamide]rRrQ) | cyclo(fffrrrrq) | cyclo(FfΦ-4gp-r-4gp-rQ) |
| cyclo(f-Dbg-rRrQ) | cyclo(FfΦRrRaQ) | cyclo(FfΦ-Cit-r-Cit-rQ) |
| cyclo(f-Bta-rRrQ) | cyclo(FfΦRrArQ) | cyclo(Tic-fΦRrRrQ) |
| cyclo(f-Bpa-rRrQ) | cyclo(FfΦRaRrQ) | cyclo(F-tic-ΦRrRrQ) |
| cyclo(fFrRrQ) | cyclo(FfΦArRrQ) | cyclo(NMeF-FΦ-NMeR-R-NMeR-RQ) |
| cyclo(fΦrRrQ)-F | cyclo(FfARrRrQ) | cyclo(FfΦRrRrQ)-F |
| cyclo(fΦrRrQ)-f | cyclo(FaΦRrRrQ) | cyclo(FfΦRrRrQ)-f |
| cyclo(fΦrRrQ)-Φ | cyclo(AfΦRrRrQ) | cyclo(FfΦRrRrQ)-Φ |
| cyclo(fΦrRrQ)-ϕ | cyclo(Cpa-fΦRrRrQ) | cyclo(FfΦRrRrQ)-ϕ |
| cyclo(fΦrRrQ)-R | cyclo(Cha-fΦRrRrQ) | cyclo(FfΦRrRrQ)-R |
| cyclo(fΦrRrQ)-r | cyclo(3,4DiF-fΦRrRrQ) | cyclo(FfΦRrRrQ)-r |
| cyclo(FFFRRRQ) | cyclo(Tfm-fΦRrRrQ) | cyclo(FfΦRrRrN)-F |
| cyclo(FRFRFRQ) | cyclo(Pfp-fΦRrRrQ) | cyclo(FfΦRrRr-hQ)-F |
| cyclo(FFΦRRRQ) | cyclo(tBuF-fΦRrRrQ) | cyclo(FfΦRrRrq)-F |

The cargo can be a protein and the EEV can be selected from:
Ac-PKKKRKV-PEG₂-K(cyclo[Ff-Nal-GrGrQ])-PEG₁₂-OH
Ac-PKKKRKV-PEG₂-K(cyclo[Ff-Nal-Cit-r-Cit-rQ])-PEG₁₂-OH
Ac-PKKKRKV-PEG₂-K(cyclo[FfF-GRGRQ])-PEG₁₂-OH
Ac-PKKKRKV-PEG₂-K(cyclo[FGFGRGRQ])-PEG₁₂-OH
Ac-PKKKRKV-PEG₂-K(cyclo[GfFGrGrQ])-PEG₁₂-OH
Ac-PKKKRKV-PEG₂-K(cyclo[FGFGRRRQ])-PEG₁₂-OH
Ac-PKKKRKV-PEG₂-K(cyclo[FGFRRRRQ])-PEG₁₂-OH
Ac-rr-PEG₂-K(cyclo[Ff-Nal-GrGrQ])-PEG₁₂-OH
Ac-rr-PEG₂-K(cyclo[Ff-Nal-Cit-r-Cit-rQ])-PEG₁₂-OH
Ac-rr-PEG₂-K(cyclo[FfF-GRGRQ])-PEG₁₂-OH
Ac-rr-PEG₂-K(cyclo[FGFGRGRQ])-PEG₁₂-OH
Ac-rr-PEG₂-K(cyclo[GfFGrGrQ])-PEG₁₂-OH
Ac-rr-PEG₂-K(cyclo[FGFGRRRQ])-PEG₁₂-OH
Ac-rr-PEG₂-K(cyclo[FGFRRRRQ])-PEG₁₂-OH
Ac-rrr-PEG₂-K(cyclo[Ff-Nal-GrGrQ])-PEG₁₂-OH
Ac-rrr-PEG₂-K(cyclo[Ff-Nal-Cit-r-Cit-rQ])-PEG₁₂-OH
Ac-rrr-PEG₂-K(cyclo[FfF-GRGRQ])-PEG₁₂-OH
Ac-rrr-PEG₂-K(cyclo[FGFGRGRQ])-PEG₁₂-OH
Ac-rrr-PEG₂-K(cyclo[GfFGrGrQ])-PEG₁₂-OH
Ac-rrr-PEG₂-K(cyclo[FGFGRRRQ])-PEG₁₂-OH
Ac-rrr-PEG₂-K(cyclo[FGFRRRRQ])-PEG₁₂-OH
Ac-rhr-PEG₂-K(cyclo[Ff-Nal-GrGrQ])-PEG₁₂-OH
Ac-rhr-PEG₂-K(cyclo[Ff-Nal-Cit-r-Cit-rQ])-PEG₁₂-OH
Ac-rhr-PEG₂-K(cyclo[FfF-GRGRQ])-PEG₁₂-OH
Ac-rhr-PEG₂-K(cyclo[FGFGRGRQ])-PEG₁₂-OH
Ac-rhr-PEG₂-K(cyclo[GfFGrGrQ])-PEG₁₂-OH
Ac-rhr-PEG₂-K(cyclo[FGFGRRRQ])-PEG₁₂-OH
Ac-rhr-PEG₂-K(cyclo[FGFRRRRQ])-PEG₁₂-OH
Ac-rbr-PEG₂-K(cyclo[Ff-Nal-GrGrQ])-PEG₁₂-OH
Ac-rbr-PEG₂-K(cyclo[Ff-Nal-Cit-r-Cit-rQ])-PEG₁₂-OH
Ac-rbr-PEG₂-K(cyclo[FfF-GRGRQ])-PEG₁₂-OH
Ac-rbr-PEG₂-K(cyclo[FGFGRGRQ])-PEG₁₂-OH
Ac-rbr-PEG₂-K(cyclo[GfFGrGrQ])-PEG₁₂-OH
Ac-rbr-PEG₂-K(cyclo[FGFGRRRQ])-PEG₁₂-OH
Ac-rbr-PEG₂-K(cyclo[FGFRRRRQ])-PEG₁₂-OH
Ac-rbrbr-PEG₂-K(cyclo[Ff-Nal-GrGrQ])-PEG₁₂-OH
Ac-rbrbr-PEG₂-K(cyclo[Ff-Nal-Cit-r-Cit-rQ])-PEG₁₂-OH
Ac-rbrbr-PEG₂-K(cyclo[FfF-GRGRQ])-PEG₁₂-OH
Ac-rbrbr-PEG₂-K(cyclo[FGFGRGRQ])-PEG₁₂-OH
Ac-rbrbr-PEG₂-K(cyclo[GfFGrGrQ])-PEG₁₂-OH
Ac-rbrbr-PEG₂-K(cyclo[FGFGRRRQ])-PEG₁₂-OH
Ac-rbrbr-PEG₂-K(cyclo[FGFRRRRQ])-PEG₁₂-OH
Ac-rbhbr-PEG₂-K(cyclo[Ff-Nal-GrGrQ])-PEG₁₂-OH
Ac-rbhbr-PEG₂-K(cyclo[Ff-Nal-Cit-r-Cit-rQ])-PEG₁₂-OH
Ac-rbhbr-PEG₂-K(cyclo[FfF-GRGRQ])-PEG₁₂-OH
Ac-rbhbr-PEG₂-K(cyclo[FGFGRGRQ])-PEG₁₂-OH
Ac-rbhbr-PEG₂-K(cyclo[GfFGrGrQ])-PEG₁₂-OH
Ac-rbhbr-PEG₂-K(cyclo[FGFGRRRQ])-PEG₁₂-OH
Ac-rbhbr-PEG₂-K(cyclo[FGFRRRRQ])-PEG₁₂-OH
Ac-hbrbh-PEG₂-K(cyclo[Ff-Nal-GrGrQ])-PEG₁₂-OH
Ac-hbrbh-PEG₂-K(cyclo[Ff-Nal-Cit-r-Cit-rQ])-PEG₁₂-OH
Ac-hbrbh-PEG₂-K(cyclo[FfF-GRGRQ])-PEG₁₂-OH
Ac-hbrbh-PEG₂-K(cyclo[FGFGRGRQ])-PEG₁₂-OH
Ac-hbrbh-PEG₂-K(cyclo[GfFGrGrQ])-PEG₁₂-OH
Ac-hbrbh-PEG₂-K(cyclo[FGFGRRRQ])-PEG₁₂-OH
Ac-hbrbh-PEG₂-K(cyclo[FGFRRRRQ])-PEG₁₂-OH
   wherein b is beta-alanine, and the exocyclic sequence can be D or L stereochemistry.

### Cargo

The cell penetrating peptide (CPP), such as a cyclic cell penetrating peptide (e.g., cCPP), can be conjugated to a cargo. The cargo can be a therapeutic moiety. The cargo can be conjugated to a terminal carbonyl group of a linker. At least one atom of the cyclic peptide can be replaced by a cargo or at least one lone pair can form a bond to a cargo. The cargo can be conjugated to the cCPP by a linker. The cargo can be conjugated to an AA_{SC} by a linker. At least one atom of the cCPP can be replaced by a therapeutic moiety or at least one lone pair of the cCPP forms a bond to a therapeutic moiety. A hydroxyl group on an amino acid side chain of the cCPP can be replaced by a bond to the cargo. A hydroxyl group on a glutamine side chain of the cCPP can be replaced by a bond to the cargo. The cargo can be conjugated to the cCPP by a linker. The cargo can be conjugated to an AA_{SC} by a linker.

The cargo can comprise one or more detectable moieties, one or more therapeutic moieties, one or more targeting moieties, or any combination thereof. The cargo can be a peptide, oligonucleotide, or small molecule. The cargo can be a peptide sequence or a non-peptidyl therapeutic agent. The cargo can be an antibody or an antigen binding fragment thereof, including, but not limited to an scFv or nanobody.

Cyclic Cell Penetrating Peptides (cCPPs) Conjugated to a Cargo Moiety

The cyclic cell penetrating peptide (cCPP) can be conjugated to a cargo moiety.

The cargo moiety can be conjugated to cCPP through a linker. The cargo moiety can comprise therapeutic moiety. The therapeutic moiety can comprise an oligonucleotide, a peptide or a small molecule. The oligonucleotide can comprise an antisense oligonucleotide. The cargo moiety can be conjugated to the linker at the terminal carbonyl group to provide the following structure:

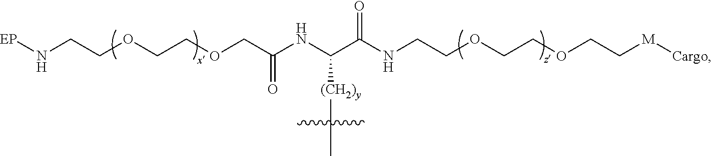

wherein:
EP is an exocyclic peptide and M, AA_{SC}, Cargo, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to a cargo to form an EEV-conjugate comprising the structure of Formula (C):

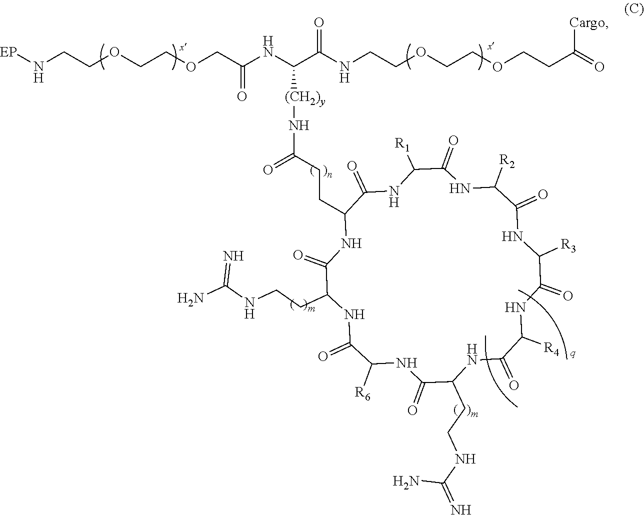

or a protonated form thereof,
wherein:
R₁, R₂, and R₃ can each independently be H or an amino acid residue having a side chain comprising an aromatic group;
R₄ is H or an amino acid side chain;
EP is an exocyclic peptide as defined herein;
Cargo is a moiety as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, cargo, m, n, x′, y, q, and z′ are as defined herein.

The EEV can be conjugated to a cargo and the EEV-conjugate can comprise the structure of Formula (C-a) or (C-b):

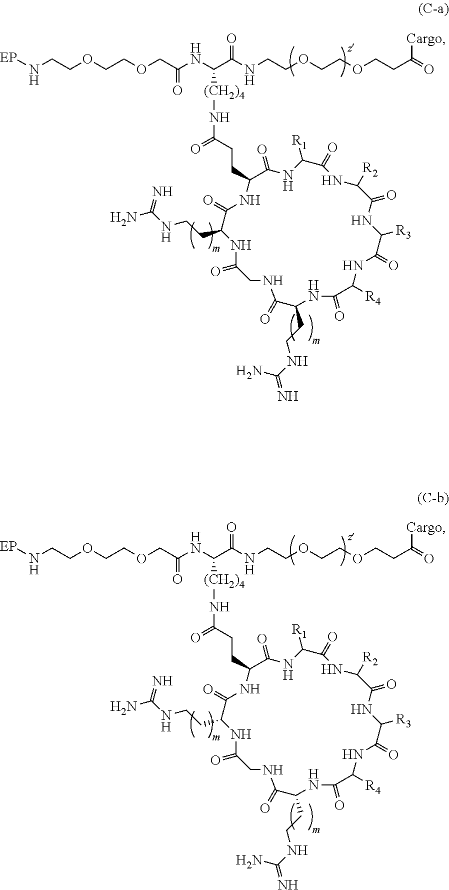

or a protonated form thereof, wherein EP, m and z are as defined above in Formula (C).

The EEV can be conjugated to a cargo and the EEV-conjugate can comprise the structure of Formula (C-c):

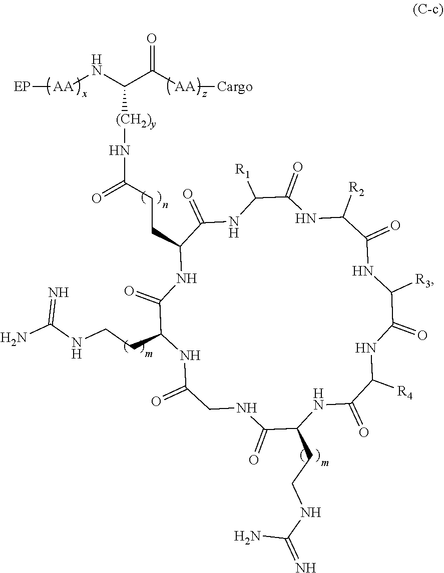

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide cargo and the EEV-oligonucleotide conjugate can comprises a structure of Formula (C-1), (C-2), (C-3), or (C-4):

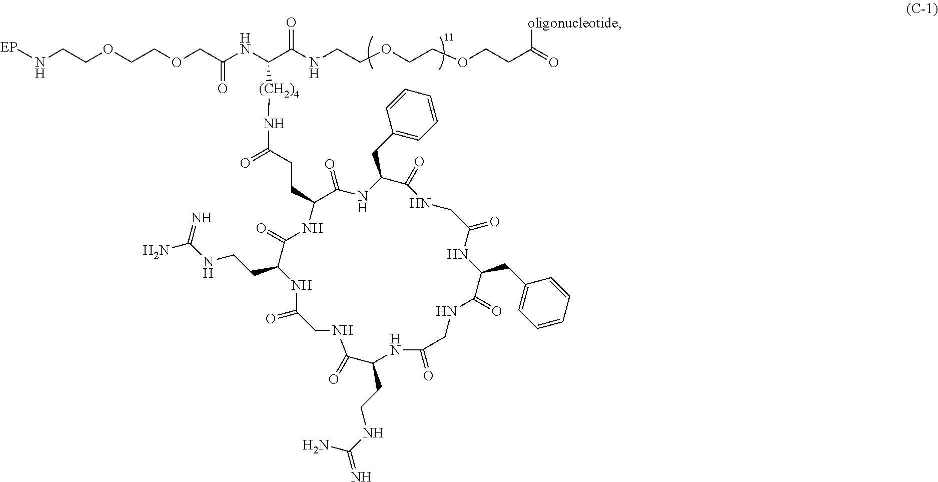

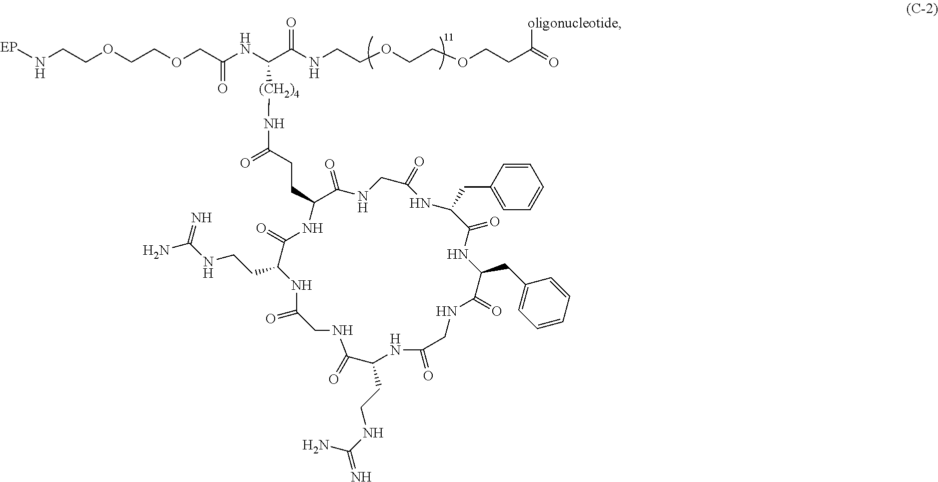

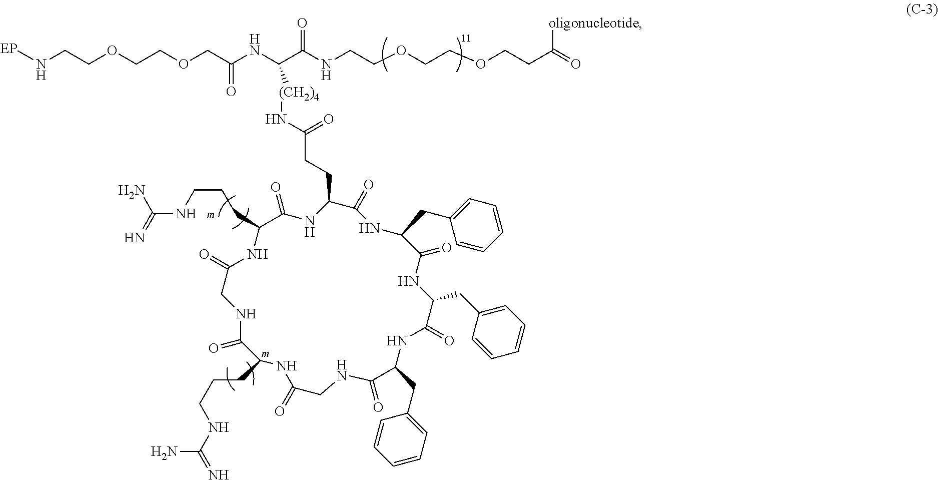

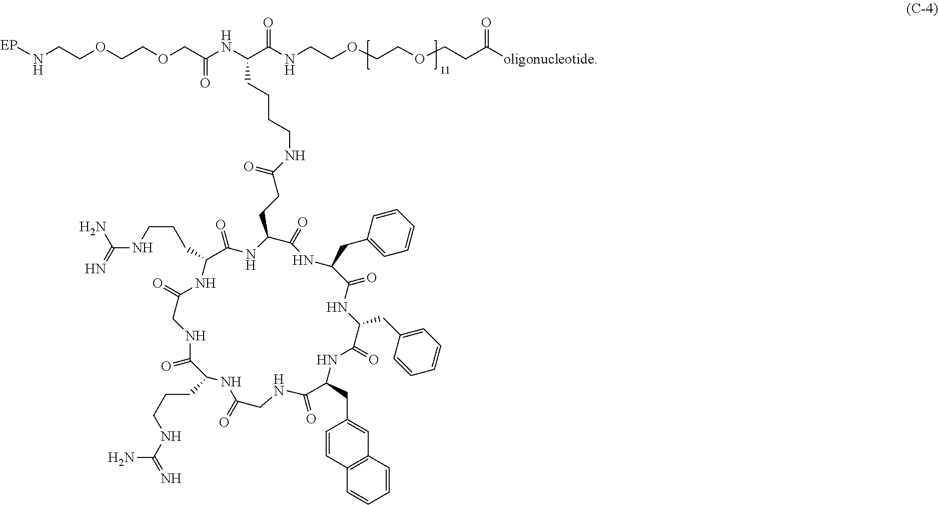

### Method of Making a Cyclic Peptide

The disclosure relates to a method of making a cyclic peptide of formula (A):

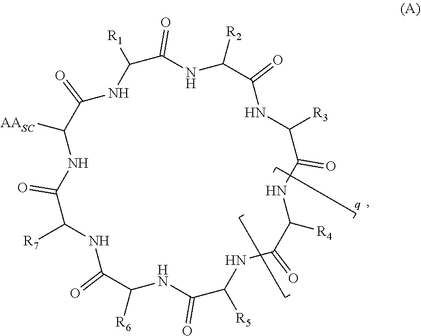

or a protonated form thereof, wherein:
R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, β-homoarginine, 3-(1-piperidinyl) alanine;
AA_{SC} is an amino acid side chain; and
q is 1, 2, 3 or 4;
wherein the method is any one of the methods herein described for a compound of Formula (I).

The disclosure relates to a method of making a cyclic peptide of Formula (I):

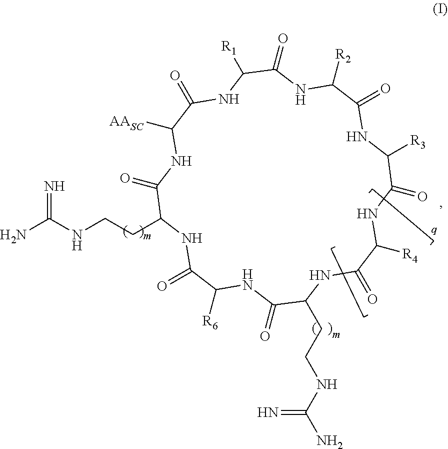

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3;
the method comprising:
reacting a compound of formula (1)

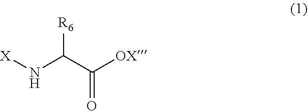

with a compound of formula (II)

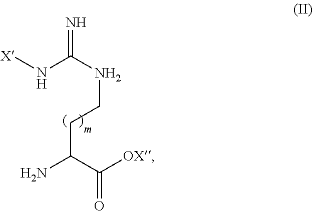

to form a compound of formula (III)

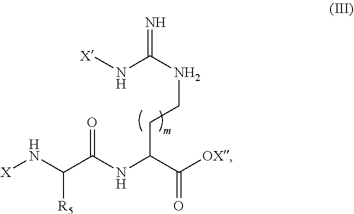

wherein X, and X′ are independently protecting groups, X″ is H or a protecting group, X′″ is H or an activating group (e.g., NHS ester), and m is 0-3. In some embodiments, X″ is methyl or t-butyl.

The method can comprise coupling or reacting a compound of formula (I) with a compound of formula (II) in the presence of a coupling reagent such as N,N′-dicyclohexylcarbodiimide (DCC). The method can further comprise treating with an activating agent such as N-hydroxysuccinimide. The method can further comprise treating with a base. The base can be NMM. In embodiments, a combination of reagent(s) and/or solvent(s) can be DCC/N-hydroxysuccinimide/THF. In embodiments, a combination of reagent(s) and/or solvent(s) can be NMM/DMF.

The method can further comprise converting a compound of formula (III) to a compound of formula (IV)

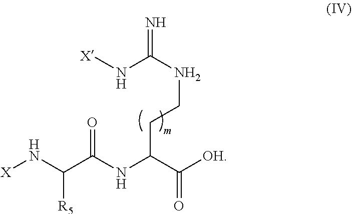

The method can comprise deprotecting or converting a compound of formula (III) to a compound of formula (IV) in the presence of base or weak acid.

The method of can also further comprise reacting a compound of formula (IV) with a compound of formula (V)

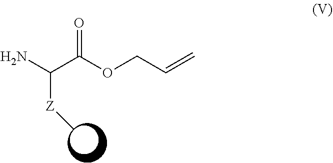

to form a compound of formula (VI)

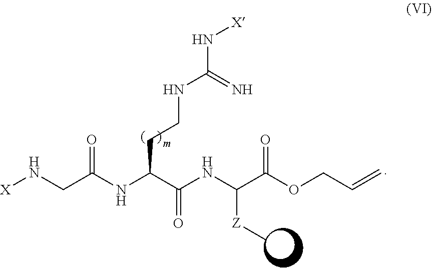

wherein Z is a radical of an amino acid side chain and

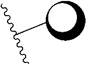

is a solid support.

The method can comprise coupling or reacting a compound of formula (VI) with a compound of formula (IV) in the presence of a coupling reagent such as DIC, HATU, DEPBT, an additive such as HOAt/Oxyma/K-Oxyma, Oxyma-B and a base such as DIPEA/NMM. In embodiments, a combination of reagent(s) and/or solvent(s) can be DIC/Oxyma. In embodiments, a combination of reagent(s) and/or solvent(s) can be DIC/HOAt. In embodiments, a combination of reagent(s) and/or solvent(s) can be DEPBT/DIPEA. In embodiments, a combination of reagent(s) and/or solvent(s) can be DEPBT/NMM. In embodiments, a combination of reagent(s) and/or solvent(s) can be HATU/NMM. In embodiments, a combination of reagent(s) and/or solvent(s) can be DIC/K-Oxyma. In embodiments, a combination of reagent(s) and/or solvent(s) can be DIC/Oxyma-B. In embodiments, the solvent comprises DMF.

The compound of formula (IV) can be

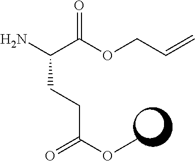

and the compound of formula (VI) can be

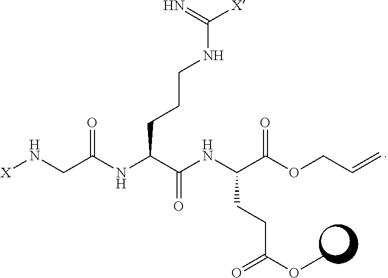

The method can further comprise treating a compound of formula (VII)

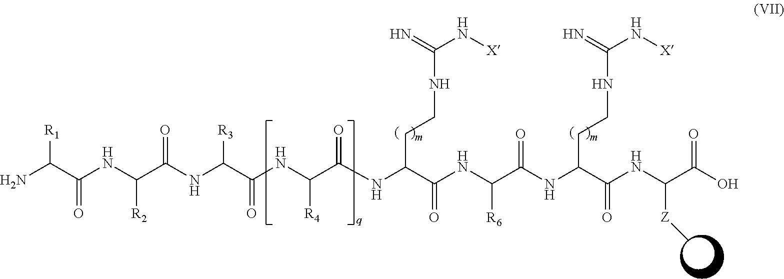

with a coupling agent, an additive and a base to obtain a compound of formula (VIII):

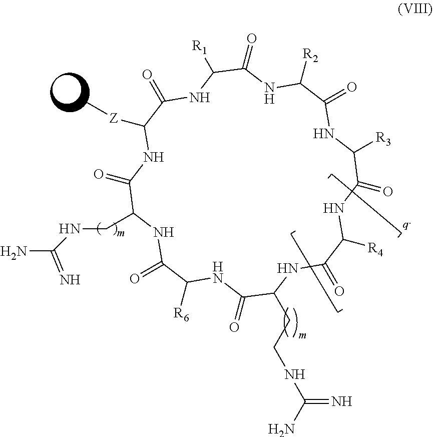

In some embodiments, the coupling agent can be PyOxim, PyAOP, PyBOP, PyBrOP, HATU, DIC, HBTU, TBTU, COMU, or DEPBT. In some embodiments, the additives can be Oxyma, HOAt, or HOBt, In some embodiments, the base can DIPEA or NMM.

In embodiments, a combination of reagent(s) and/or solvent(s) can be HATU/HOAt/DIPEA. In embodiments, a combination of reagent(s) and/or solvent(s) can be PyAOP/HOAt/DIPEA. In embodiments, a combination of reagent(s) and/or solvent(s) can be PyAOP/HOAt/NMM. In embodiments, a combination of reagent(s) and/or solvent(s) can be PyBOP/HOBt/DIPEA. In embodiments, a combination of reagent(s) and/or solvent(s) can be PyBrop/DIPEA. In embodiments, a combination of reagent(s) and/or solvent(s) can be PyOxim/Oxyma/DIPEA. In embodiments, a combination of reagent(s) and/or solvent(s) can be DIC/HOBt/DIPEA. In embodiments, a combination of reagent(s) and/or solvent(s) can be HBTU/HOBt/DIPEA. In embodiments, a combination of reagent(s) and/or solvent(s) can be TBTU/HOBt/DIPEA. In embodiments, a combination of reagent(s) and/or solvent(s) can be COMU/Oxyma/DIPEA. In embodiments, a combination of reagent(s) and/or solvent(s) can be DEPBT/DIPEA. In embodiments, the solvent comprises DMF.

The compound of formula (VII) can be

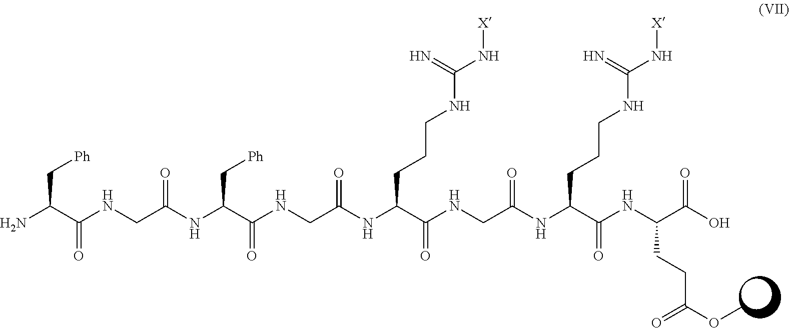

and the compound of formula (VIII) can be

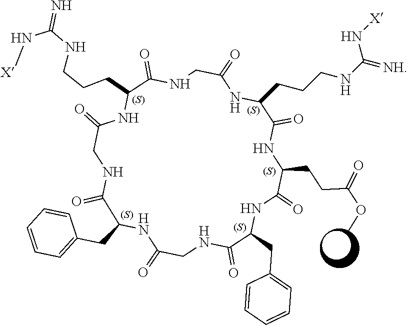

The disclosure also relates to a method of making a cyclic peptide of Formula (Ia):

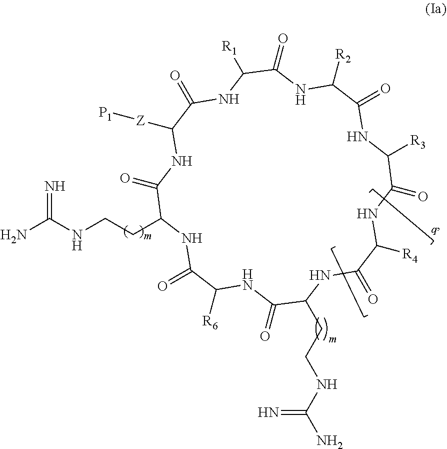

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
Z is radical of an amino acid side chain;
P₁ is

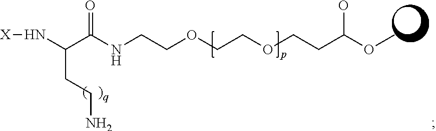

q is 1, 2, 3 or 4; and
each m is independently an integer 0, 1, 2, or 3,
the method comprising:
reacting a compound of formula (IX)

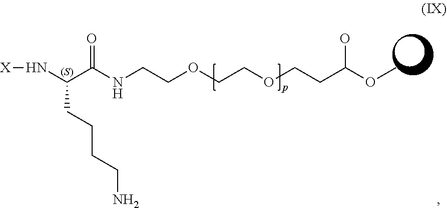

wherein X is a protecting group and

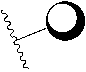

is a solid support, with a compound of formula (X)

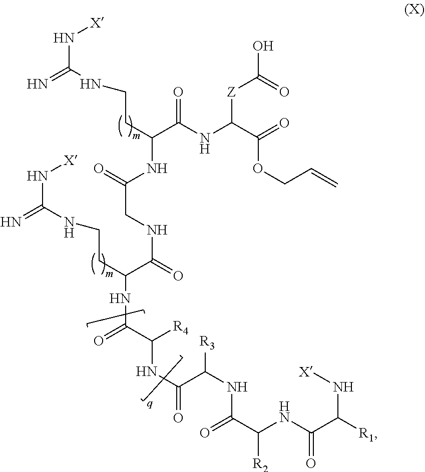

wherein X′ are each independently protecting groups and Z is a radical of an amino acid side chain, to form a compound of formula (XI)

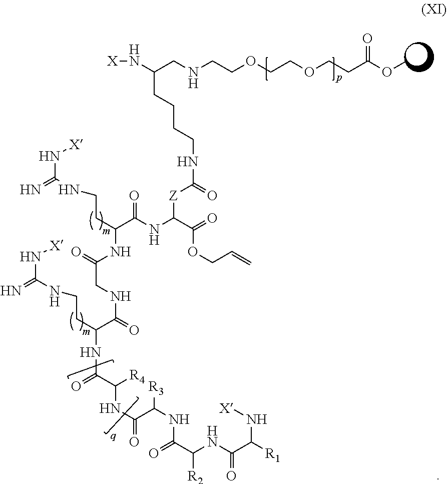

The method can comprise coupling or reacting a compound of formula (IX) with a compound of formula (X) in the presence of standard solid phase peptide conditions [Chan, W. C., White P. D., ed. Fmoc Solid Phase Peptide Synthesis: A Practical Approach, Oxford University Press, 2000],

The compound of formula (X) can be

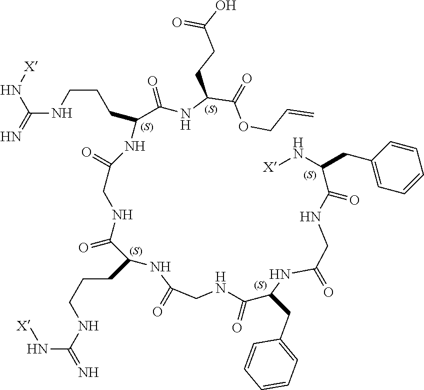

and the compound of formula (XI) can be

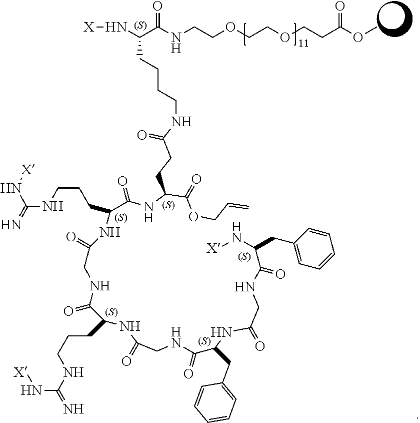

The method can further comprise treating the compound of formula (XI) to form a compound of formula (XII)

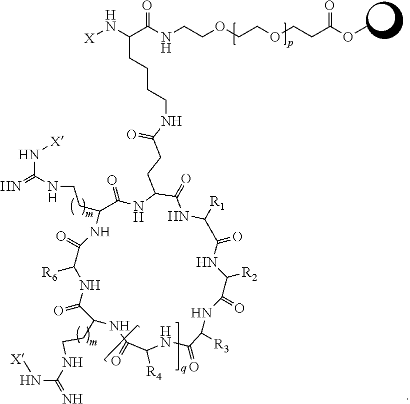

The method can comprise treat a compound of formula (XI) with a compound of formula (XII) in the presence of bases such as piperidine/hydrazine/DBU/sodium hydroxide/pyrrolidine/morpholine/diethylamine/tert-butylamine. The method can further comprising adding Pd(PPh₃)₄/PhSiH₃/DCM. The method can further comprising adding a coupling reagent such as PyOxim, an additive such as Oxyma, and a base such as DIPEA.

In embodiments, a combination of reagent(s) and/or solvent(s) can be piperidine. In embodiments, a combination of reagent(s) and/or solvent(s) can be piperidine/formic acid. In embodiments, a combination of reagent(s) and/or solvent(s) can be piperidine/Oxyma. In embodiments, a combination of reagent(s) and/or solvent(s) can be DBU. In embodiments, a combination of reagent(s) and/or solvent(s) can be DBU/piperidine. In embodiments, a combination of reagent(s) and/or solvent(s) can be DBU/piperidine/Oxyma. In embodiments, a combination of reagent(s) and/or solvent(s) can be DBU/piperidine/HOBt. In embodiments, a combination of reagent(s) and/or solvent(s) can be DBU/piperazine/formic acid. In embodiments, a combination of reagent(s) and/or solvent(s) can be tert-butyl amine, pyrrolidine. In embodiments, a combination of reagent(s) and/or solvent(s) can be morpholine. In embodiments, a combination of reagent(s) and/or solvent(s) can be diethylamine. In embodiments, a combination of reagent(s) and/or solvent(s) can be sodium hydroxide. In embodiments, a combination of reagent(s) and/or solvent(s) can be Pd(PPh₃)₄/PhSiH₃/DCM for allyl ester removal. In embodiments, a combination of reagent(s) and/or solvent(s) can be PyOxim/Oxyma/DIPEA/DMF/DCM for cyclization.

The compound of formula (XII) can be

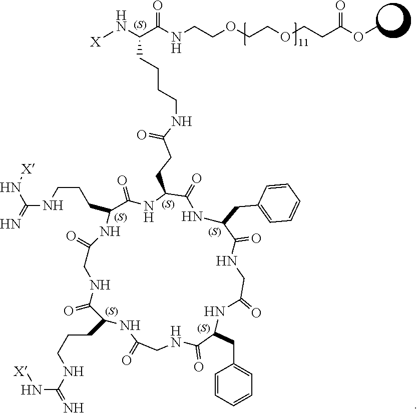

The disclosure relates to a method of making a cyclic peptide of Formula (I):

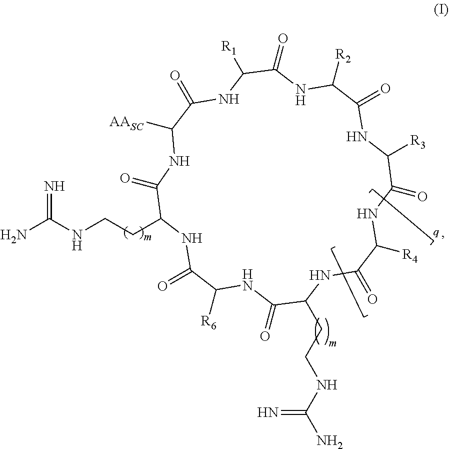

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3;
the method comprising: reacting a compound of formula (XIII)

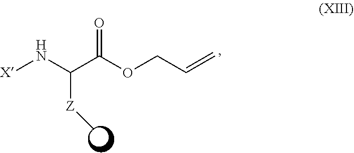

wherein X′ is a protecting group, Z is a radical of an amino acid side chain and

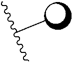

is a solid support, with a compound of formula (XIV)

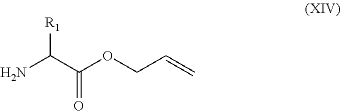

to give a compound of formula (XV)

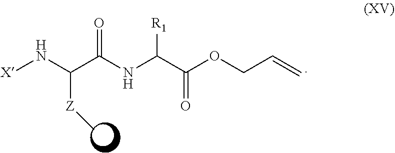

The method can comprise coupling or reacting a compound of formula (XIII) with a compound of formula (XIV) in the presence of Pd(PPh₃)₄/PhSiH₃/DCM to remove the allyl ester and subsequently assembled according to standard solid phase peptide synthesis conditions for deprotection and coupling of amino acids [Chan, W. C., White P. D., ed. Fmoc Solid Phase Peptide Synthesis: A Practical Approach, Oxford University Press, 2000]

The method wherein the compound of formula (XIII) is

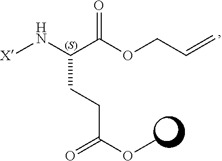

the compound of formula (XIV) is

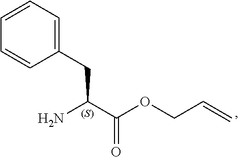

and the compound of formula (XV) is

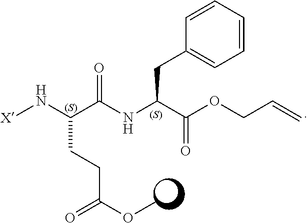

The method can further comprise treating the compound of formula (XV) to obtain a compound of formula (XVI):

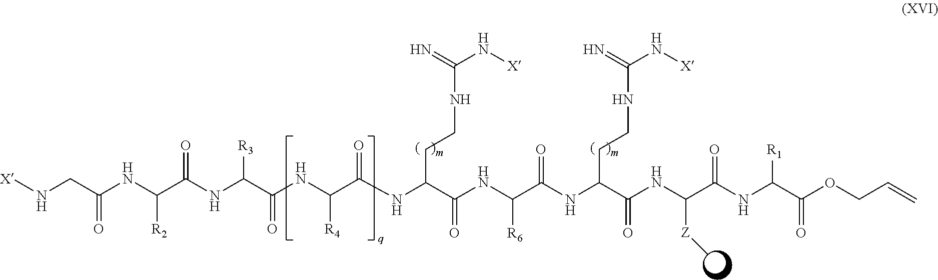

The method can comprise treating a compound of formula (XV) in the presence of standard solid phase peptide synthesis conditions for deprotection and coupling of amino acids [Chan, W. C., White P. D., ed. Fmoc Solid Phase Peptide Synthesis: A Practical Approach, Oxford University Press, 2000].

The compound of formula (XVI) can be

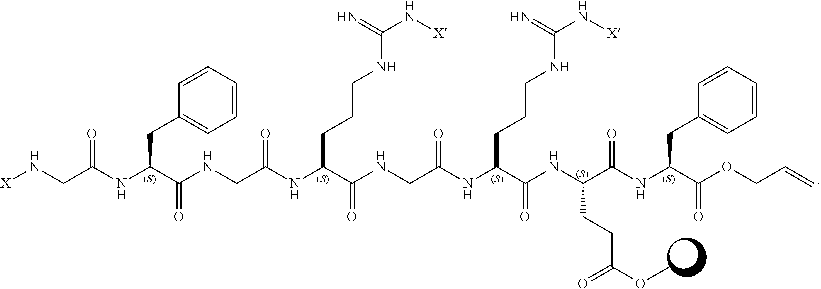

### Alternative Cyclic Peptide Formation

The disclosure also relates to making a compound of formula (D)

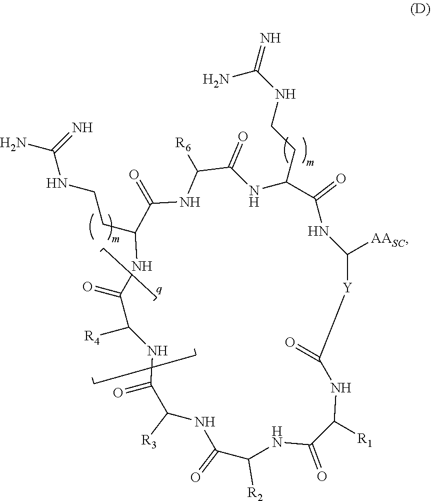

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
Y is

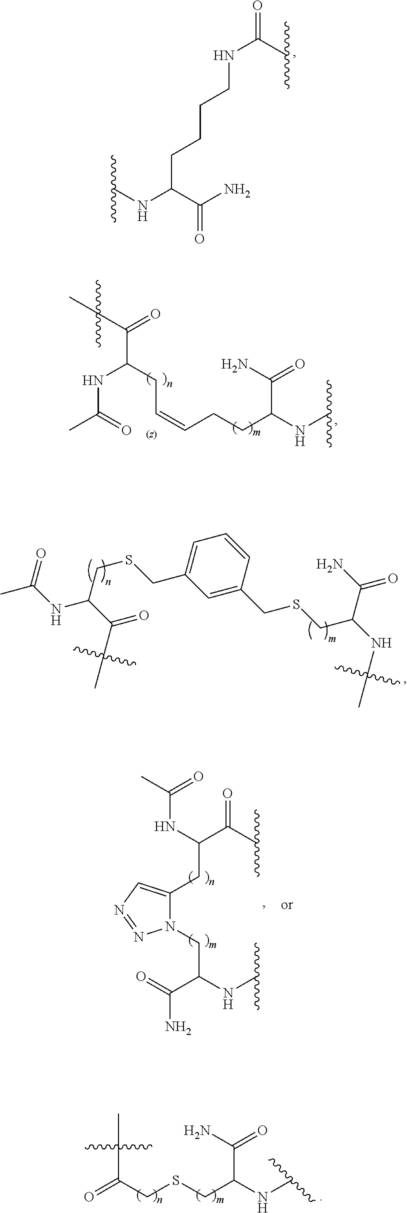

q is 1, 2, 3 or 4;
each m is independently an integer 0, 1, 2, or 3, and
each n is independently an integer 0, 1, 2, or 3.

### Macrolactamization

The disclosure also relates to a method of making a cyclic peptide of Formula (D-I):

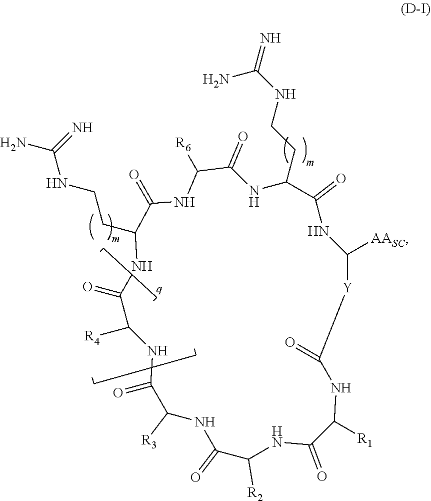

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and
Y is

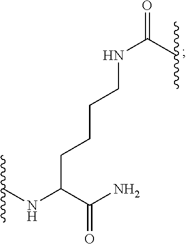 [SMILES: *NC(CCCCNC(*)=O)C(N)=O]

the method comprising: cyclizing a compound of Formula (XVII)

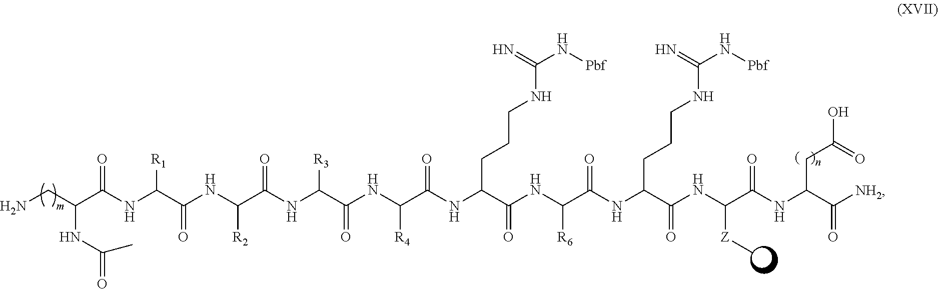

wherein Z is a radical of an amino acid side chain and

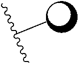

is a solid support.

An example of a synthetic scheme is illustrated in Scheme 1.

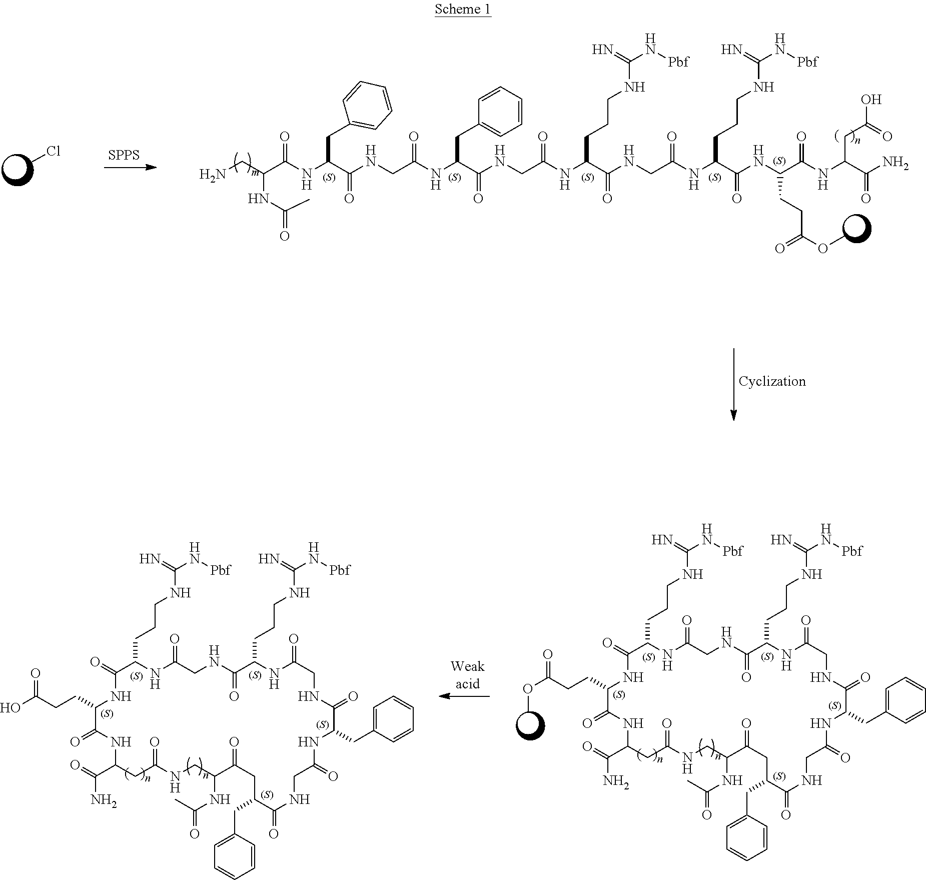

The method can comprise standard solid phase peptide synthesis conditions for deprotection and coupling of amino acids [Chan, W. C., White P. D., ed. Fmoc Solid Phase Peptide Synthesis: A Practical Approach, Oxford University Press, 2000]. For example, the use of a coupling reagent such as PyOxim, an additive such as Oxyma and a base such as DIPEA for cyclization, and treatment with for example HFIP or TFA for cleavage from the solid.

### Ring Closing Metathesis (RCM)

The disclosure also relates to a method of making a cyclic peptide of Formula (D-II):

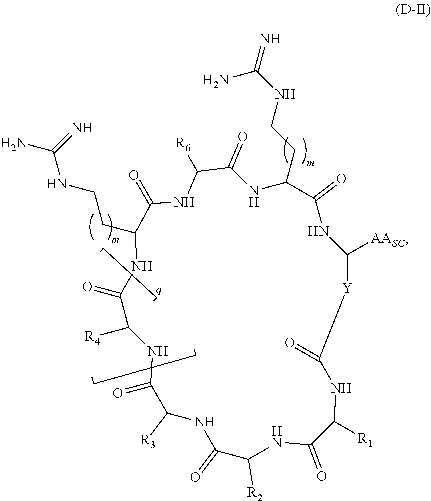

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3,
each n is independently an integer 0, 1, 2, or 3, and
Y is

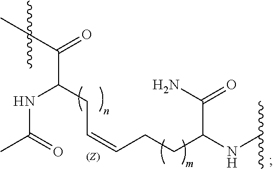

the method comprising: cyclizing a compound of Formula (XVIII):

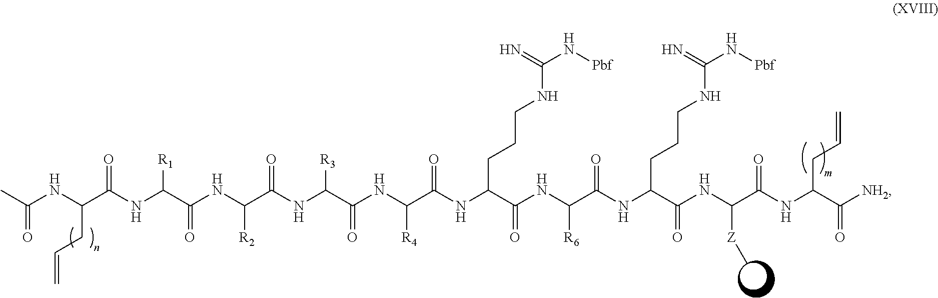

wherein Z is a radical of an amino acid side chain and

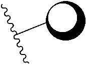

is a solid support.

An example of a synthetic scheme is illustrated in Scheme 2.

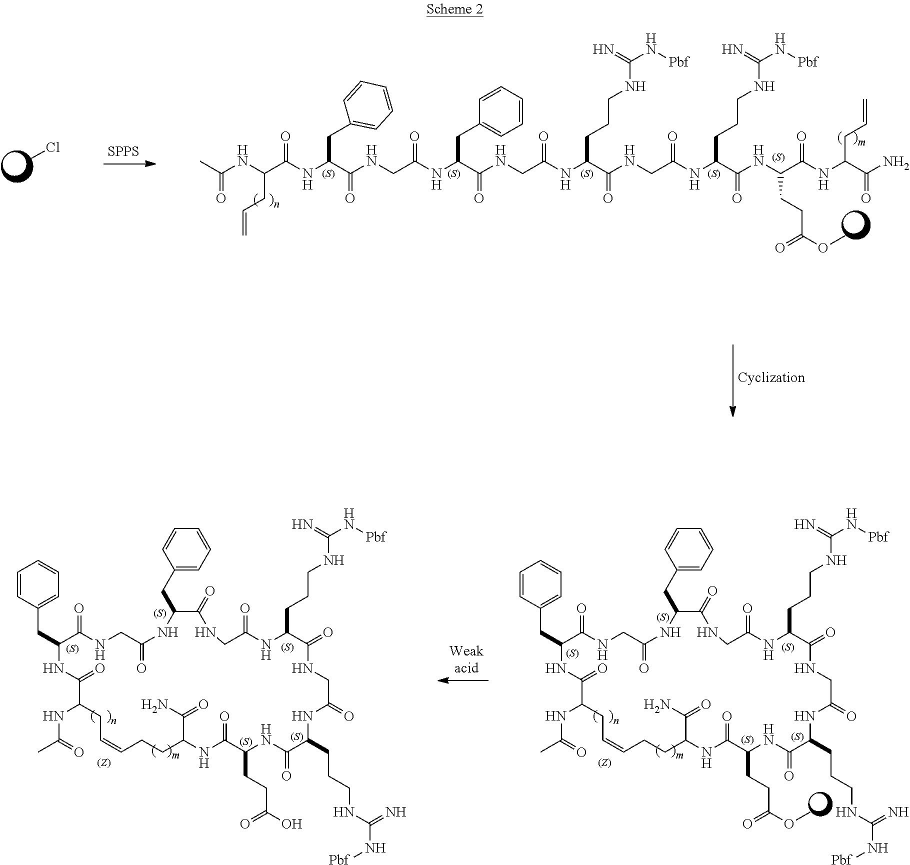

The method can comprise standard solid phase peptide synthesis conditions for deprotection and coupling of amino acids [Chan, W. C., White P. D., ed. Fmoc Solid Phase Peptide Synthesis: A Practical Approach, Oxford University Press, 2000]. For example, the use of a coupling reagent such as PyOxim, an additive such as Oxyma and a base such as DIPEA for cyclization, and treatment with for example HFIP or TFA for cleavage from the solid.

### Thioester Stapling

The disclosure also relates to a method of making a cyclic peptide of Formula (D-III):

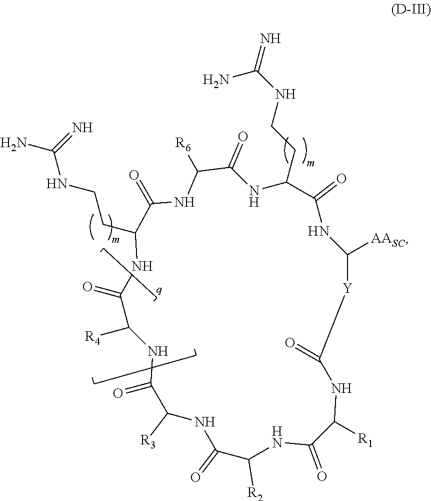

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3,
each n is independently an integer 0, 1, 2, or 3, and
Y is

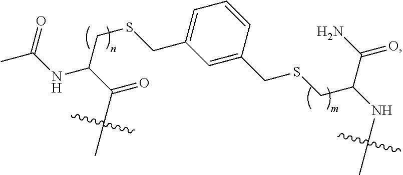

the method comprising: cyclizing a compound of Formula (XIX):

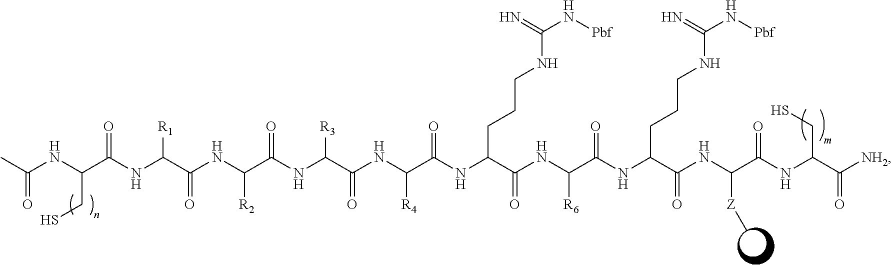

wherein Z is a radical of an amino acid side chain and

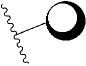

is a solid support.

An example of a synthetic scheme is illustrated in Scheme 3.

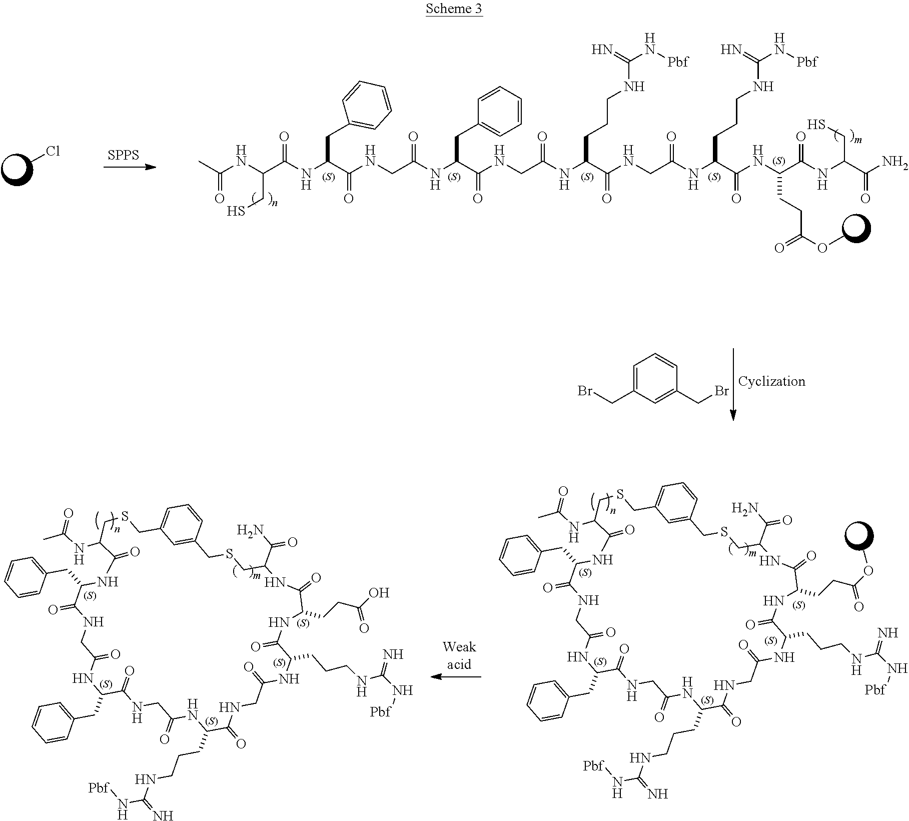

The method can comprise standard solid phase peptide synthesis conditions for deprotection and coupling of amino acids [Chan, W. C., White P. D., ed. Fmoc Solid Phase Peptide Synthesis: A Practical Approach, Oxford University Press, 2000]. For example, the use of a coupling reagent such as PyOxim, an additive such as Oxyma and a base such as DIPEA for cyclization, and treatment with for example HFIP or TFA for cleavage from the solid.

### Cu(I)-Catalyzed Azide-Alkyne Cycloaddition (CuAAC)

The disclosure also relates to a method of making a cyclic peptide of Formula (D-IV):

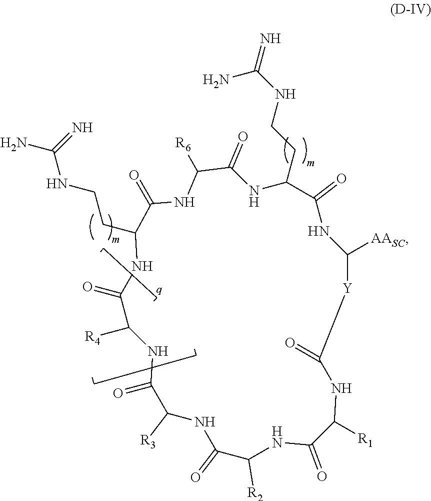

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and
Y is

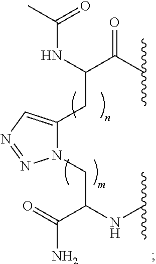

the method comprising: cyclizing a compound of Formula (XX):

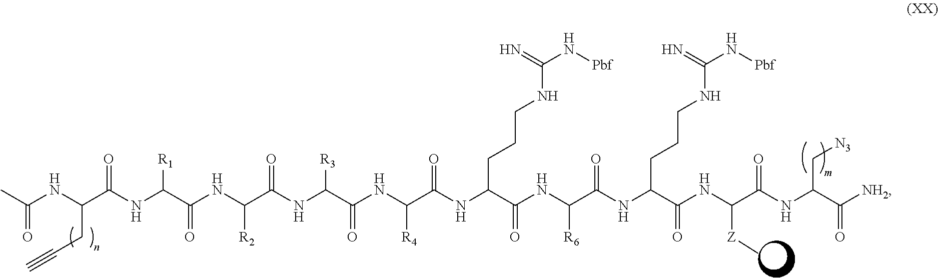

wherein Z is a radical of an amino acid side chain and

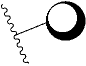

is a solid support.

An example of a synthetic scheme is illustrated in Scheme 4.

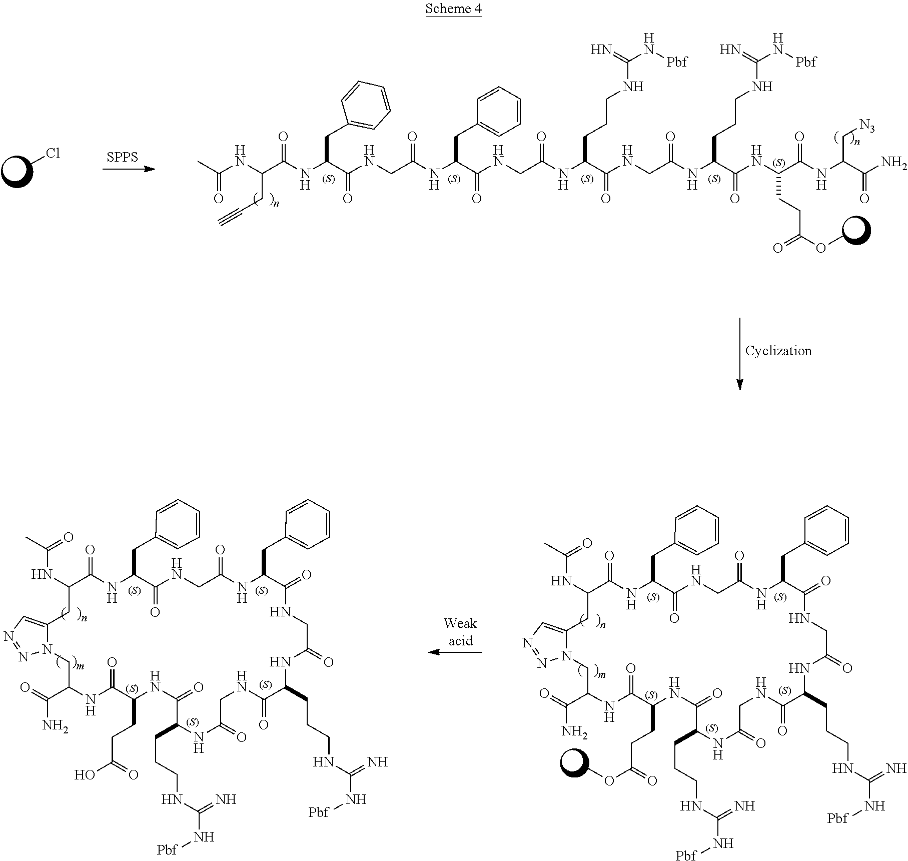

The method can comprise standard solid phase peptide synthesis conditions for deprotection and coupling of amino acids [Chan, W. C., White P. D., ed. Fmoc Solid Phase Peptide Synthesis: A Practical Approach, Oxford University Press, 2000].

For example, the use of a coupling reagent such as PyOxim, an additive such as Oxyma and a base such as DIPEA for cyclization, and treatment with for example HFIP or TFA for cleavage from the solid.

### Thioether Cyclization

The disclosure also relates to a method of making a cyclic peptide of Formula (D-V):

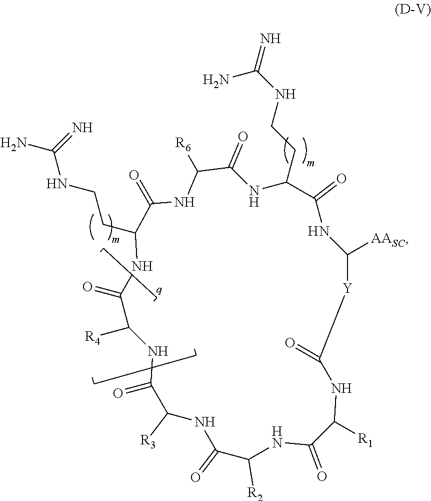

or a protonated form thereof, wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and
Y is

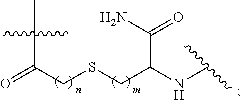 [SMILES: *NC(CSCC(*)=O)C(N)=O]

the method comprising: cyclizing a compound of Formula (XXI)

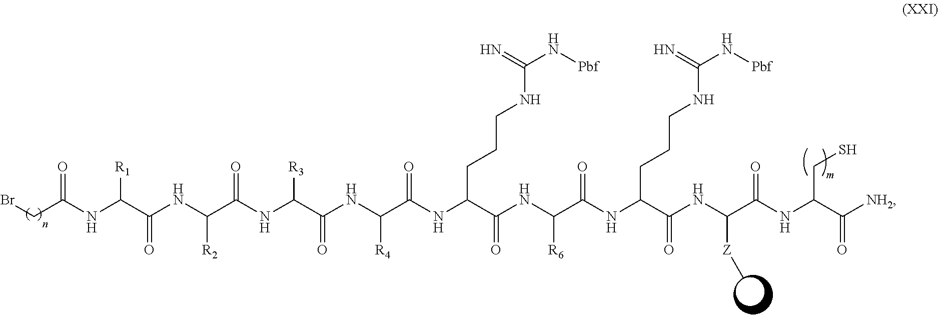

wherein Z is a radical of an amino acid side chain and

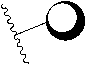

is a solid support.

An example of a synthetic scheme is illustrated in Scheme 5.

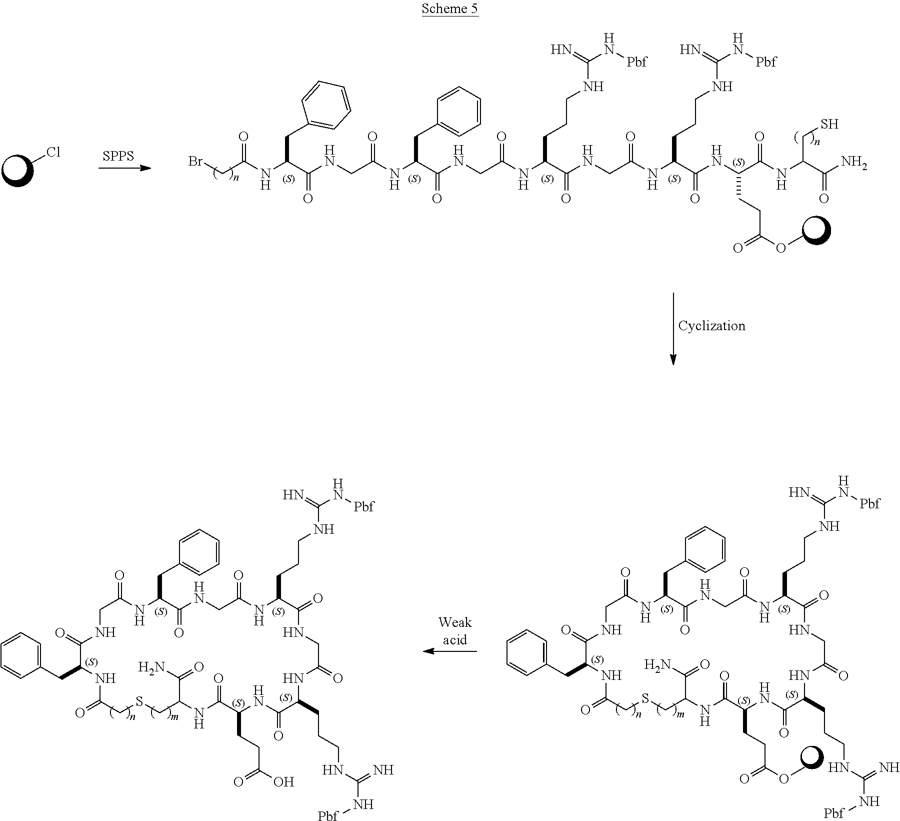

The method can comprise standard solid phase peptide synthesis conditions for deprotection and coupling of amino acids [Chan, W. C., White P. D., ed. Fmoc Solid Phase Peptide Synthesis: A Practical Approach, Oxford University Press, 2000]. For example, the use of a coupling reagent such as PyOxim, an additive such as Oxyma and a base such as DIPEA for cyclization, and treatment with for example HFIP or TFA for cleavage from the solid.

Coupling Cyclic Peptide onto Linear Peptide

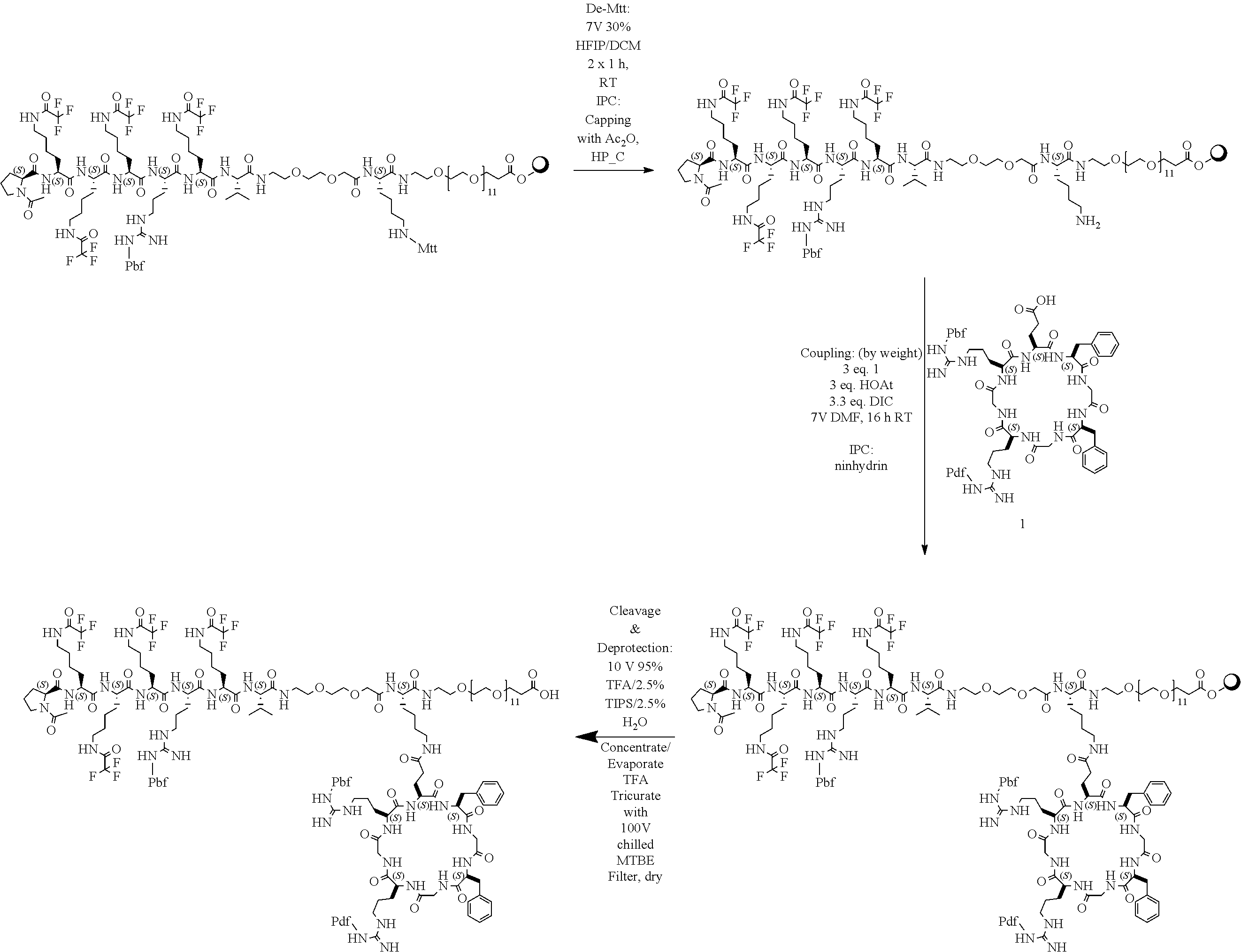

### Method of Making Phosphorodiamidate Morpholino Oligomer (PMO)

PMO can be made according to any method known in the art, such as illustrated in Summerton et al. U.S. Pat. No. 5,166,315. Nov. 24, 1992; Summerton et al. U.S. Pat. No. 5,185,444, Feb. 9, 1993; Summerton et al. U.S. Pat. No. 5,217,866, Jun. 8, 1993; Summerton et al. U.S. Pat. No. 5,235,033. Aug. 10, 1993; Summerton et al. U.S. Pat. No. 5,506,337, Apr. 9, 1996; Summerton et al. U.S. Pat. No. 5,521,063, May 28, 1996; Summerton et al. Antisense Nucleic Acid Drug Dev. 1997, 7:187-195; Iversen, P. International Patent WO 02/092617 A1, Nov. 21, 2002; Stein et al. U.S. Pat. No. 6,828,105 B2, Dec. 7, 2004; Iversen et al. US Patent Application 2005/0261249 A1, Nov. 24, 2005; Mourich et al. US Patent Application 2006/0276425 A1, Dec. 7, 2006; Stein et al. US Patent Application 2007/0004661 A1, Jan. 4, 2007; Stein et al. US Patent Application 2007/0129323 A1, Jun. 7, 2007; Moulton et al. International Patent WO 2009/005793 A2, Jan. 8, 2009; Moulton et al. US Patent Application 2010/0016215 A1, Jan. 21, 2010; Sazani et al. US Patent Application, 2010/0130591, May 27, 2010; Weller et al. US Patent Application 2010/0234281 A1, Sep. 16, 2010; Weller et al. U.S. Pat. No. 7,943,762, B2, May 17, 2011; Weller et al. U.S. Pat. No. 8,067,571 B2, Nov. 29, 2011, Reeves et al. U.S. Pat. No. 8,076,476 B2, Dec. 13, 2011; Fox et al. U.S. Pat. No. 8,299,206 B2, Oct. 30, 2012; Linsley et al. United States Patent Application 2014/030238, Oct. 9, 2014; Linsley et al. United States Patent Application 2014/0329772, Nov. 6, 2014; Ueda, T. U.S. Pat. No. 8,969,551 B2, Mar. 3, 2015; Hanson, G. U.S. Pat. No. 9,161,948, Oct. 20, 2015; Bhadra et al Nucleic Acid Chem. 2015, 62:4.65.1-6.65.26; Totaro et al. International Patent WO 2019/0-60862 A1, Mar. 28, 2019, Torii et al. U.S. Pat. No. 10,415,036 B2, Sep. 17, 2019; Cai et al. US Patent Application 2019/0292208 A1, Sep. 26, 2019; Bestwick et al. US Patent Application 2019/0365918 A1, Dec. 5, 2019; Passini et al. US Patent Application 2020/0377886 A1, Dec. 3, 2020; Sinha et al. US Patent Application 2021/0130379, May 6, 2021; Fang et al. International Patent WO 2022/125987 A1, and in Scheme 6.

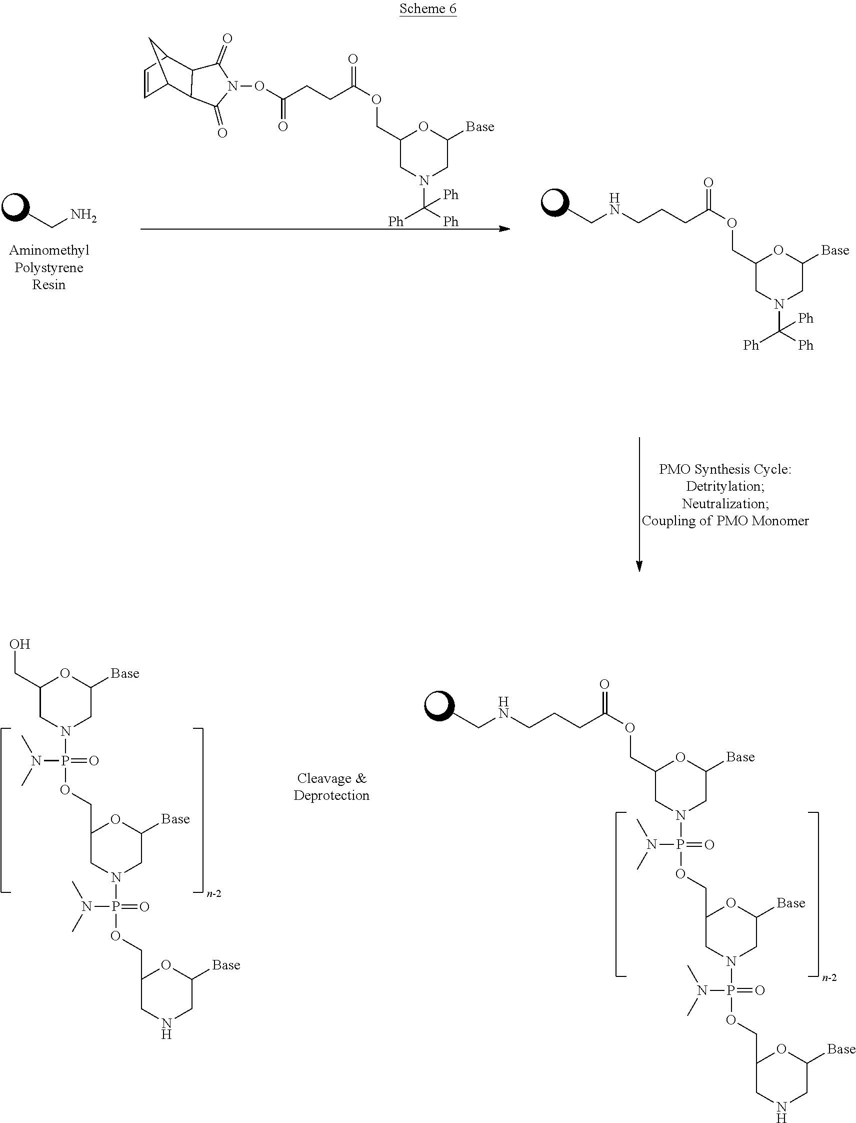

The method can comprise for example, treating with 4-cyanopyridine and TFA for detritylation, DIPEA for neutralization, and adding PMO monomers in the presence of a base such as NEM for coupling. The method can comprise for example, further treating with DTT in the presence of a base such as DBU for cleavage, and further with a base such as ammonium hydroxide for deprotection.

### Method of Conjugation Peptide+PMO

A cyclic peptide can be conjugated to a PMO according to any method known in the art, such as illustrated in [Hanson, G. Peptide Oligonucleotide Conjugates. U.S. Pat. No. 9,161,948 B2, Oct. 20, 2015] and in Scheme 7. Various reaction condition showing activation of the N3 terminal peptide by treatment with a base and a “coupling reagent” followed by addition of the PMO are also illustrated in Example 4.

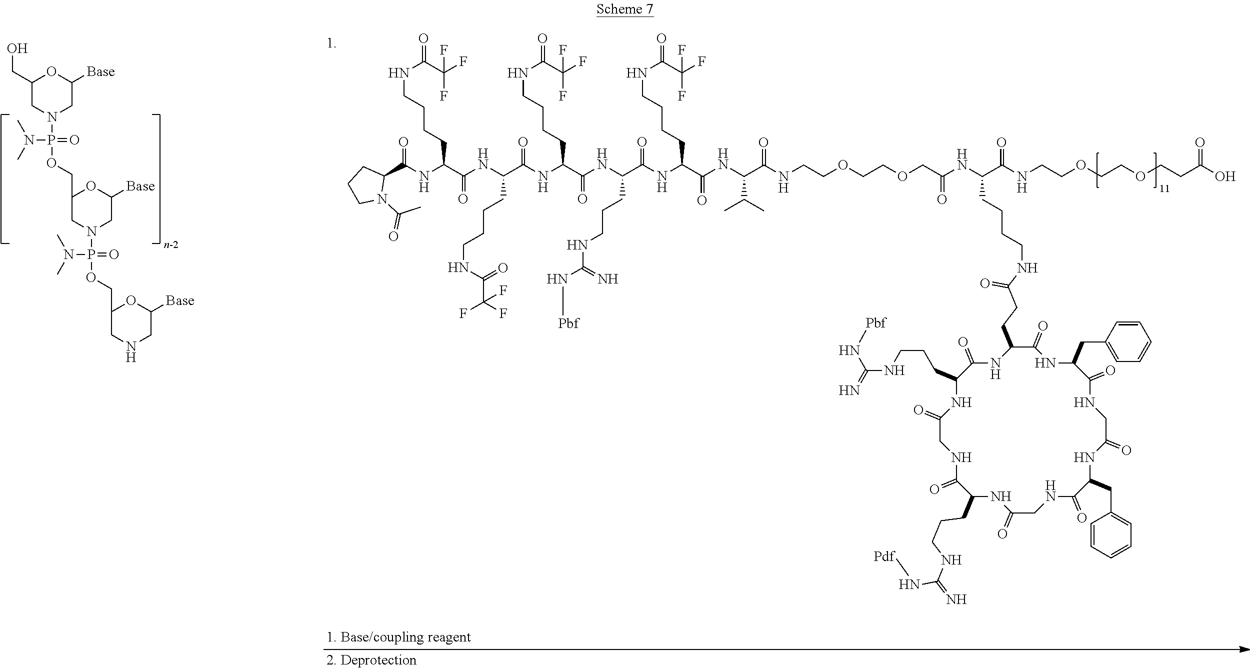

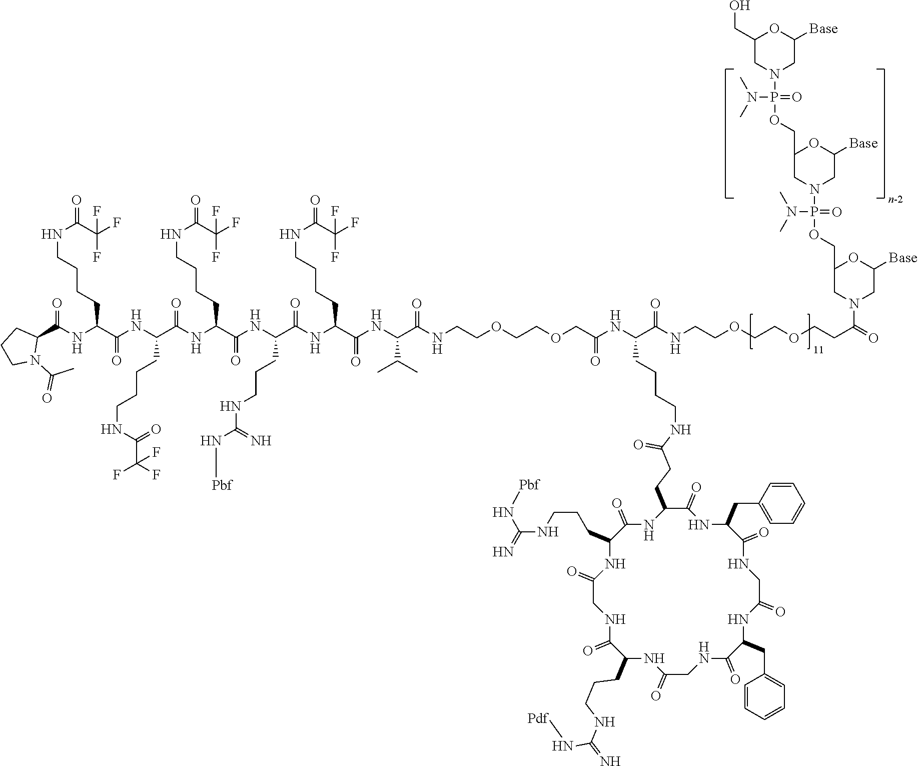

The method can comprise treating with a coupling reagent such as DIC/HATU/PyAOP, an additive such as Oxyma and a base such as DIPEA. The method can comprise treating with a base such as sodium hydroxide/lithium hydroxide/potassium hydroxide/potassium carbonate/potassium chloride for deprotection.

### Compounds

The disclosure also relates to a compound selected from

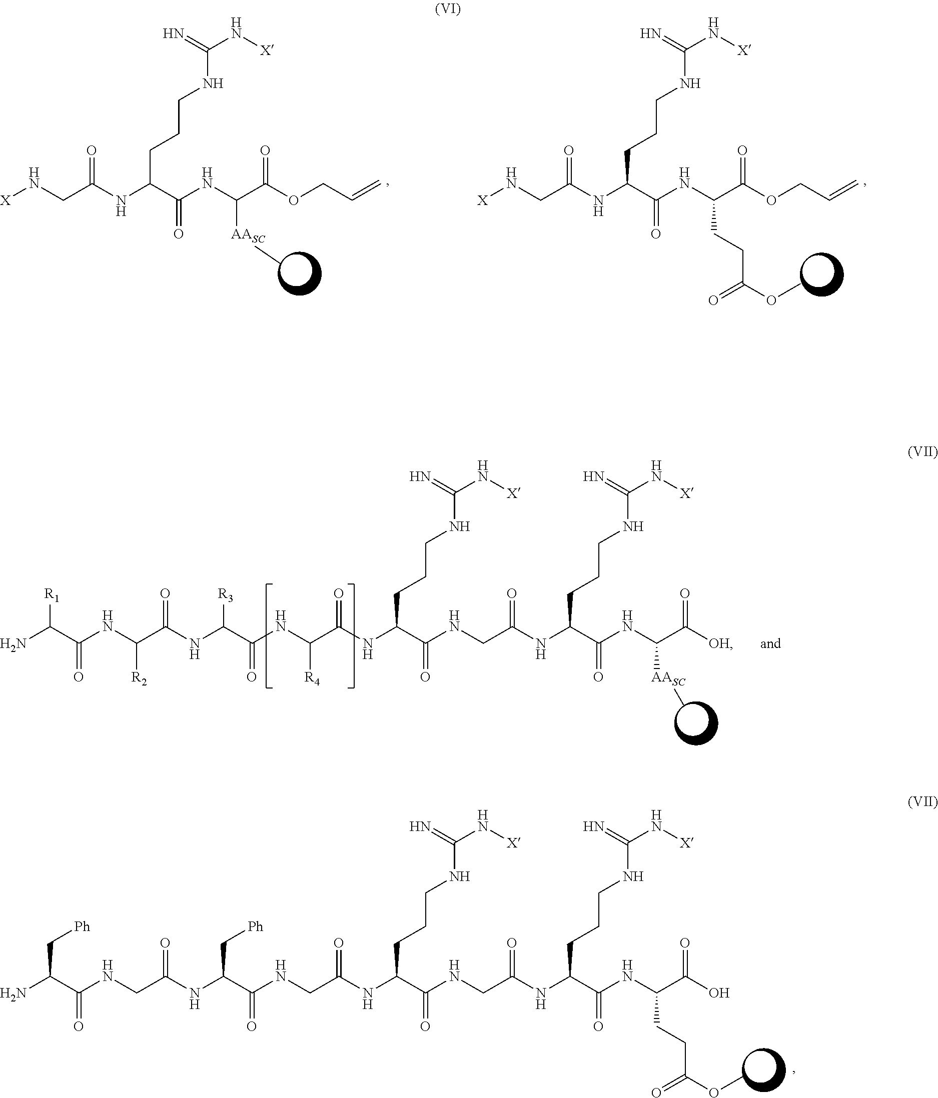

wherein:
R₁, R₂, and R₃ are each independently H or a side chain of tyrosine, phenylalanine or tryptophan;
   R₄ is H or an amino acid side chain;
   AA_{SC} is an amino acid side chain;
   q is 1, 2, 3 or 4;
   X, X′, and X″ are each independently protecting groups;
   each m is independently an integer from 0-3; and

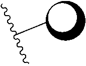

is a solid support.

The disclosure also relates to a compound selected from

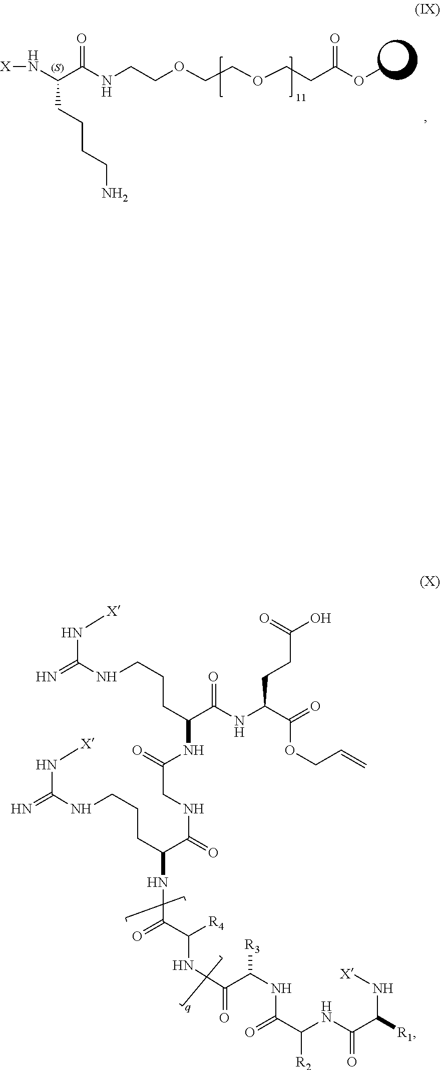

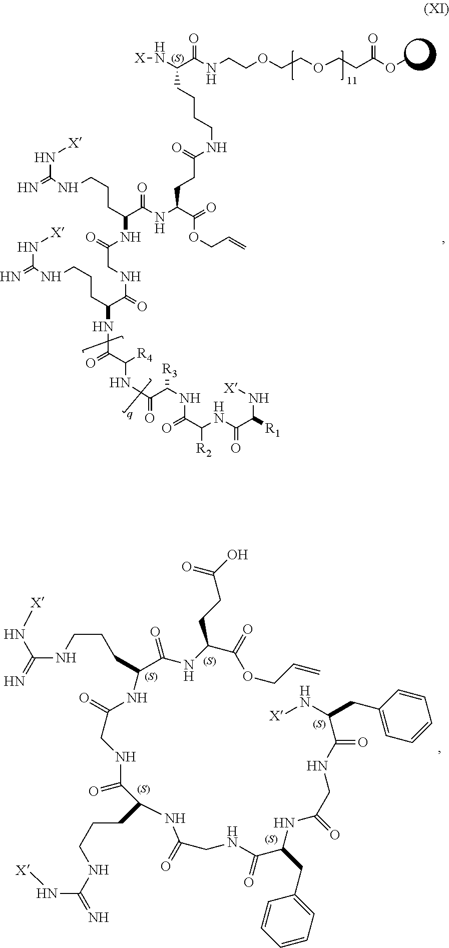

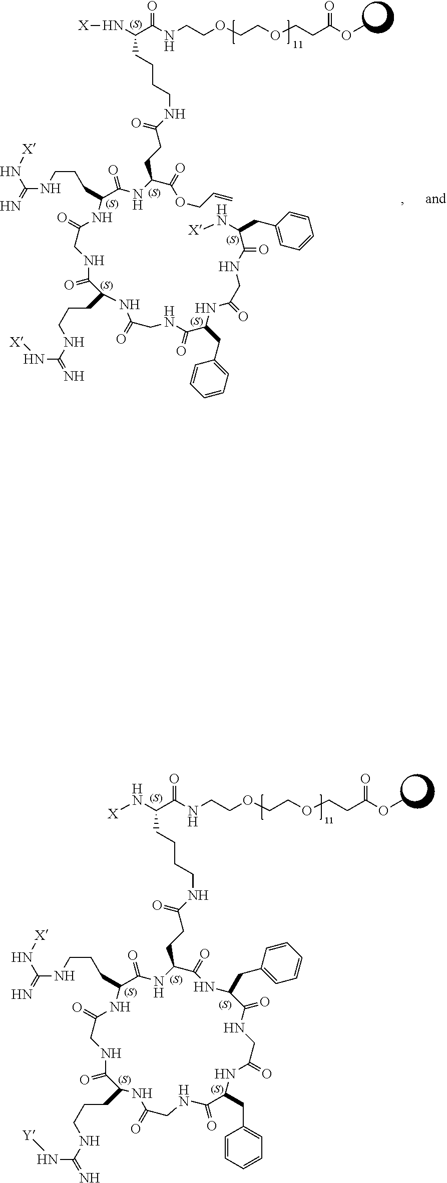

wherein:
R₁, R₂, and R₃ are each independently H or a side chain of tyrosine,
   phenylalanine or tryptophan;
   R₄ is H or an amino acid side chain;
   AA_{SC} is an amino acid side chain;
   q is 1, 2, 3 or 4
   X and X′ are each independently protecting groups;
   each m is independently an integer from 0-3; and

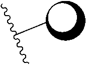

is a solid support.

The disclosure also relates to a compound selected from

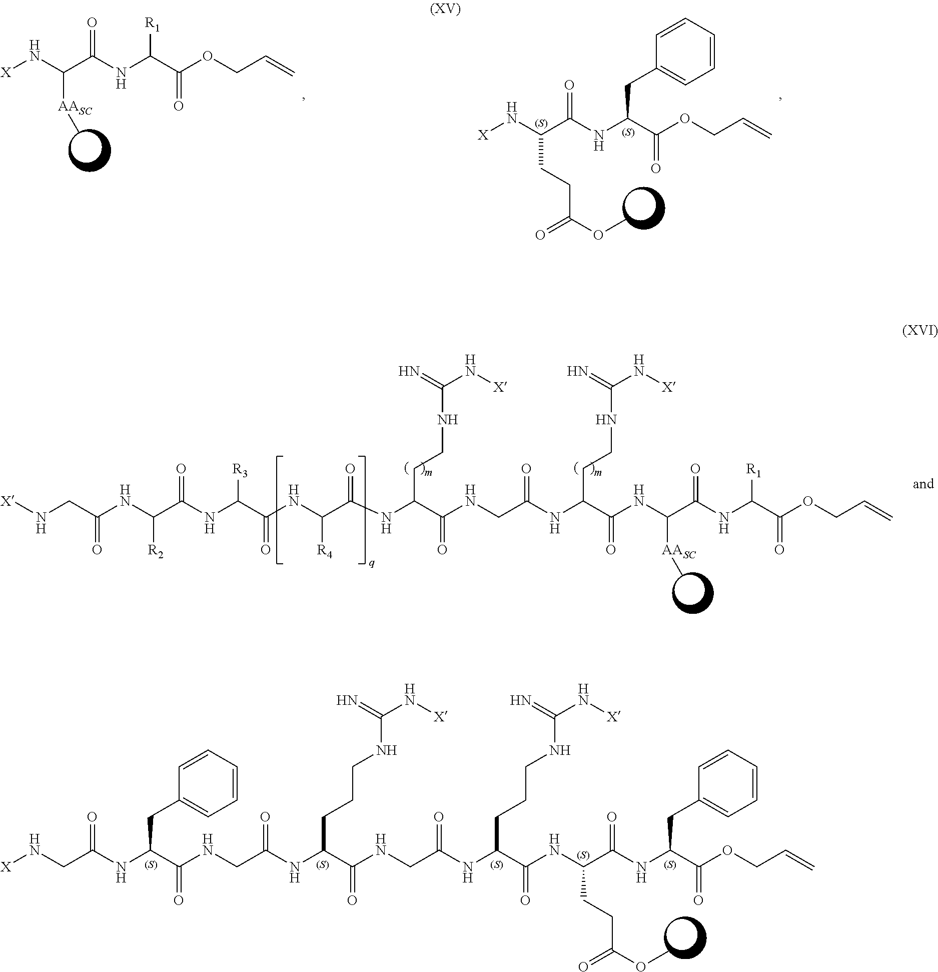

wherein:
R₁, R₂, and R₃ are each independently H or a side chain of tyrosine, phenylalanine or tryptophan;
   R₄ is H or an amino acid side chain;
   AA_{SC} is an amino acid side chain;
   q is 1, 2, 3 or 4
   X, and X′ are each independently protecting groups;
   each m is independently an integer from 0-3; and

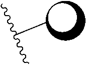

is a solid support.

The synthesis can be performed manually or be automated or a combination of both.

The resin loading level can be ˜0.1-1.0 mmol/g. The resin loading level can be ˜0.30-0.50 mmol/g. The resin loading level can be ˜0.20-0.30 mmol/g. The resin loading level can be ˜0.20-0.25 mmol/g. The resin loading level can be ˜0.2-0.50 mmol/g. The resin loading level can be ˜0.2-0.60 mmol/g. The resin loading level can be ˜0.2-0.70 mmol/g. The resin loading level can be ˜0.2-0.80 mmol/g. The resin loading level can be ˜0.2-0.90 mmol/g. The resin loading level can be ˜0.22-0.92 mmol/g. The resin loading level can be ˜0.22 mmol/g. The resin loading level can be ˜0.39 mmol/g. The resin loading level can be ˜0.46 mmol/g. The resin loading level can be ˜0.64 mmol/g. The resin loading level can be ˜0.77 mmol/g. The resin loading level can be ˜0.92 mmol/g.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” (also written as “˜”) when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

“2-[2-[2-aminoethoxy]ethoxy]acetic acid” is also referred to as AEEA or miniPEG.

As used herein, the term “cyclic cell penetrating peptide” or “CPP” refers to a peptide that facilitates the delivery of a cargo, e.g., a therapeutic moiety, into a cell.

As used herein, the term “endosomal escape vehicle” (EEV) refers to a CPP that is conjugated by a chemical linkage (i.e., a covalent bond or non-covalent interaction) to a linker as defined herein and/or an exocyclic peptide as defined herein. The EEV of the present disclosure is an EEV of Formula (B).

As used herein, the term “EEV-conjugate” refers to an endosomal escape vehicle defined herein conjugated by a chemical linkage (i.e., a covalent bond or non-covalent interaction) to a cargo. The cargo can be a therapeutic moiety (e.g., an oligonucleotide) that can be delivered into a cell by the EEV. The EEV-conjugate of the present disclosure can be an EEV-conjugate of Formula (C).

As used herein, the term “exocyclic peptide” (EP) and “modulatory peptide” (MP) may be used interchangeably to refers to two or more amino acid residues linked by a peptide bond that can be conjugated to a cyclic peptide disclosed herein. The EP, when conjugated to a cyclic peptide disclosed herein, alters the tissue distribution and/or retention of the compound. Typically, the EP can comprise at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV, the nucleoplasmin bipartite NLS with the sequence NLSKRPAAIKKAGQAKKKK, the c-myc nuclear localization sequence having the amino acid sequence PAAKRVKLD or RQRRNELKRSF, the sequence RMRKFKNKGKDTAELRRRRVEVSVELRKAKKDEQILKRRNV of the IBB domain from importin-alpha, the sequences VSRKRPRP and PPKKARED of the myoma T protein, the sequence PQPKKKPL of human p53, the sequence SALIKKKKKMAP of mouse c-abl IV, the sequences DRLRR and PKQKKRK of the influenza virus NS1, the sequence RKLKKKIKKL of the Hepatitis virus delta antigen and the sequence REKKKFLKRR of the mouse Mxl protein, the sequence KRKGDEVDGVDEVAKKKSKK of the human poly (ADP-ribose) polymerase and the sequence RKCLQAGMNLEARKTKK of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and a cargo, e.g., an oligonucleotide, peptide or small molecule) to the cyclic peptide. The linker can comprise a natural or non-natural amino acid or polypeptide. The linker can be a synthetic compound containing two or more appropriate functional groups suitable to bind cyclic peptide to a cargo moiety, to thereby form the compounds disclosed herein. The linker can comprise a polyethylene glycol (PEG) moiety. The linker can comprise one or more amino acids. For example, the cyclic peptide may be covalently bound to a cargo via a linker.

As used herein, the term “oligonucleotide” refers to an oligomeric compound comprising a plurality of linked nucleotides or nucleosides. In One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

The term “therapeutic polypeptide” refers to a polypeptide that has therapeutic, prophylactic or other biological activity. The therapeutic polypeptide can be produced in any suitable manner. For example, the therapeutic polypeptide may isolated or purified from a naturally occurring environment, may be chemically synthesized, may be recombinantly produced, or a combination thereof.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

As used herein, the term “contiguous” refers to two amino acids, which are connected by a covalent bond. For example, in the context of a representative cyclic peptide such as

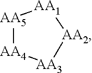

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic peptides described herein have amino acids (e.g., arginine) incorporated therein through formation of one or more peptide bonds. The amino acids incorporated into the cyclic peptide may be referred to residues, or simply as an amino acid. Thus, arginine or an arginine residue refers to

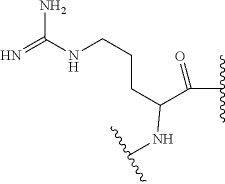 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

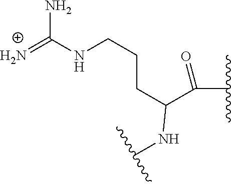 [SMILES: *NC(CCCNC(N)=[NH2+])C(*)=O]

As used herein, the term “chirality” refers to the “D” and “L” isomers of amino acids or amino acid residues.

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein below.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein “activating group” is meant an electron donating group that increases the stability and overall reactivity of the compound/intermediate. An activating group can be for example NHS ester or PhSiH₃.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

| Abbreviation | IUPAC Name |
|---|---|
| HATU | 1-[Bis(dimethylamino)methylene]-1H- |
| | 1,2,3-triazolo[4,5-b]pyridinium 3-oxide |
| | hexafluorophosphate |
| HOAt | 3H-[1,2,3]Triazolo[4,5-b]pyridin-3-ol |
| HOBt | 1H-1,2,3-Benzotriazol-1-ol |
| PyAOP | (7-Azabenzotriazol-1- |
| | yloxy)tripyrrolidinophosphonium |
| | hexafluorophosphate |
| PyOxim | [(E)-(1-cyano-2-ethoxy-2- |
| | oxoethylidene)amino]oxy-tripyrrolidin- |
| | 1-ylphosphanium; hexafluorophosphate |
| Oxyma | Ethyl (2Z)-2-cyano-2- |
| | (hydroxyimino)acetate |
| Oxyma-B | 5-(hydroxyimino)-1,3- |
| | dimethylpyrimidine-2,4,6(1H,3H,5H)- |
| | trion |
| K-Oxyma | Z-ethyl (hydroxyimino)cyanoacetate |
| | potassium salt |
| HBTU | 3-[Bis(dimethylamino)methyliumyl]- |
| | 3H-benzotriazol-1-oxide |
| | hexafluorophosphate |
| TBTU | 2-(1H-Benzotriazole-1-yl)-1,1,3,3- |
| | tetramethyluronium tetrafluoroborate |
| COMU | (1-Cyano-2-ethoxy-2- |
| | oxoethylidenaminooxy)dimethylamino- |
| | morpholino-carbenium |
| | hexafluorophosphate |
| DEPBT | Diethyl 4-oxo-1,2,3-benzotriazin- |
| | 3(4H)-yl phosphate |
| DCC | N,N′-Dicyclohexylmethanediimine |
| NMM | 4-methylmorpholine |
| NEM | 4-ethylmorpholine |
| DIPEA | N-ethyl-N-propan-2-ylpropan-2-amine |
| DTT | (2S,3S)-1,4-Bis-sulfanylbutane-2,3- |
| | diol |
| DBU | 2,3,4,6,7,8,9,10- |
| | Octahydropyrimido[1,2-a]azepine |
| DMF | N,N-dimethylformamide |
| DCM | Dichloromethane |
| HFIP | 1,1,1,3,3,3-hexafluoropropan-2-ol |
| NMP | 1-Methylpyrrolidin-2-one |
| DMI | 1,3-Dimethylimidazolidin-2-one |
| IPA | Propan-2-ol |
| TFE | 2,2,2-Trifluoroethan-1-ol |
| THF | Oxacyclopentane |
| TIPS | Tri(propan-2-yl)silane |
| TFA | Trifluoroacetic acid |
| MBTE | 2-methoxy-2-methylpropane |
| DCBC | 1,3-dichloro-2-(chloromethyl)benzene |
| PYR | pyridine |

### EXAMPLES

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art. All reactions can be carried out in solution (use of a solvent or mixture of solvents) or neat (no solvent needed).

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

### Example 1: Linear Peptide Synthesis (Linker Synthesis)

Linear Peptide Synthesis-Loading of Fmoc-PEG₁₂-CH₂CH₂COOH onto Wang Resin

To a glass peptide synthesis vessel containing Wang Resin, add DCM, swell the resin and drain the solvent. Add DCM to the resin. Weigh desired amount of Fmoc-PEG₁₂-CH₂CH₂COOH into a container and dissolve in a minimal amount of DMF and transfer the resulting solution to the resin vessel and add pyridine. Add 2,6-dichlorobenzoyl chloride and stir the resin suspension until the reaction is determined to be complete.

### Fmoc Deprotection

Add DMF to a glass peptide synthesis vessel containing resin-bound peptide, swell and drain. Add 20% piperidine in DMF solution to fully cover resin and react. Drain and sequentially wash with DMF, DCM, and again DMF.

### Coupling of Each Amino Acids (AA)

Weigh Fmoc-AA into a container and add additive (e.g., Oxyma. Dissolve Fmoc-AA and additive in DMF to completely dissolve. Add DIC, mix, and allow to stand at room temperature. Add the pre-activated Fmoc-AA solution onto resin and mix

### Example 2: Cyclic Peptide Synthesis

Loading of First Fmoc-AA onto 2-Chlorotrityl Chloride (CTC) Resin

To a glass peptide synthesis vessel containing CTC resin, add DMF and swell the resin and drain the solvent. Weigh desired Fmoc-AA and dissolve in DMF. Add the AA solution to the resin. Add DIPEA and mix. Once the reaction is determined to be complete, quench with methanol and wash the resin.

Fmoc Deprotection: as described in Example 1

### Methyltrityl (Mtt) Deprotection for Exocyclic Peptide

Prepare a solution of hexafluoroisopropanol (HFIP) in DCM. Add it to the vessel containing resin-bound peptide. Mix the contents at room temperature. Drain and wash the resin with DCM. Repeat until deprotection is complete.

Coupling of each AAₛ: as described in Example 1

### Allyl Ester Deprotection for Cyclic Peptide

Add resin bed volumes of DCM to a glass peptide synthesis vessel containing the linear precursor of the cyclic peptide that is Fmoc-protected on the N-terminus and Allyl-protected on the C-terminus. Swell the resin at room temperature. Weigh Pd(PPh₃)₄ and dissolve the solid in DCM. Drain the resin and add fresh DCM. Add PhSiH₃ to the resin suspended in DCM. Add Pd solution to resin, protect from light, and mix at room temperature. Drain the resin and sequentially wash with DCM and DMF. Weigh out SDDC·(H₂O)₃ (sodium diethyldithiocarbamate trihydrate) and dissolve in DMF. Add the mixture to the resin and mix. Once complete, wash resin with DCM and DMF.

Cleavage from Solid Support-Cyclic Peptide

Prepare a mixture of HFIP: DCM and add to the peptide synthesis vessel containing resin-bound cyclic peptide and mix. Wash the resin with minimum amount of DCM. Evaporate the filtrate to a minimum volume and triturate with cold MTBE. Filter the resulting suspension and wash the cake with MTBE.

### Example 3: Coupling of the Cyclic Peptide with a Linear Peptide

Dissolve crude cyclic peptide and HOAt in DMF. Add DIC, mix, and allow to stand at RT. Add the pre-activated cyclic peptide solution onto resin and mix. Shake the reaction mixture at room temperature.

Cleavage from Solid Support

Prepare a mixture of TFA:DCM 95:5 (v/v) and add to the glass peptide synthesis vessel containing resin-bound cyclic peptide. Mix the contents and filter. Wash the resin with minimum amount of DCM. Evaporate the filtrate to a minimum volume and triturate with cold MTBE. Filter the resulting suspension and wash the cake with MTBE. The resulting crude peptide can be purified by preparative RP-HPLC and further lyophilized.

### Example 4: Phosphorodiamidate Morpholino Oligomer (PMO) Synthesis

Loading PMO Monomer onto Solid Support:

Suspend aminomethyl polystyrene resin in NMP and swell the resin. Filter the resin to remove NMP and wash with DCM, 5% DIPEA solution in DCM, and DCM. Dissolve the PMO Monomer, functionalized as an activated succinate ester in dry NMP and add to the resin. Stir the reaction mixture at RT. Filter the resin and wash with NMP and DCM. Dry the resin and test the loading by trityl quantitation. Prepare a solution of 0.4 M benzoic anhydride and NEM in NMP solution. Add the benzoic anhydride/NEM solution to the resin and stir at RT. Filter the resin and wash with NMP two times, IPA two times, and DCM two times. Dry the resin.

### Determining Resin Loading by Trityl Quantitation

Place dry resin (50-100 mg) in a fritted syringe and swell with of DCM. Remove the DCM and add deprotection solution (3% TFA in DCM) to resin. Agitate the solution for 5 min and collect the filtrate. Repeated this process for another four times until the solution is colorless. Combine all filtrates, dilute the solution with water and measure the absorption value of sample solution at 404 nm. Use 3% TFA in DCM as the blank while testing. Use L=OD*N*12/ε/W to determine resin loading (L: Loading of resin, mmol/g; OD: OD value of sample solution; N: Dilution factor; W: Weight of the resin, g; ¿: Molar absorption coefficient=32500)

### Resin Swelling for PMO Synthesis

Before initiating PMO synthesis, the resin functionalized with PMO Monomer was suspended in NMP and swelled for 2 h.

### Trityl Deprotection

Wash resin with DCM. Treat the resin with 4-cyanopyridine, TFA in 80:20:1 DCM/TFE/EtOH (CYTFA) solution

### Neutralization

Treat the resin with a neutralization solution containing 5% of DIPEA in 1:3 IPA/DCM. Wash the resin with DCM and anhydrous (1,3-Dimethyl-2-imidazolidinone) DMI.

### Coupling

Prepare a solution containing 0.2 M PMO monomer and 0.4 M NEM in anhydrous DMI. For the first coupling, use 5 eq. of PMO monomer and of NEM. For the next couplings, up to the 10th position of the sequence, use 3 eq. of PMO monomer and 6 eq. of NEM. For the couplings from the 10th up to the 20th position, use 4 eq. of PMO monomer and 8 eq. of NEM. For the couplings from the 20th position onward, use 5 eq. of PMO monomer and 10 eq. of NEM. Add the coupling solution to resin and react at RT to 45° C. Coupling reactions performed at 45° C. were found to be more efficient and are complete in a shorter timeframe (2-4 h). Monitor coupling reactions by chloranil test (Pept Res., 8 (4): 236-7. 1995). Wash the resin with DCM and 30% TFE in DCM. The resin can be stored overnight in 30% TFE in DCM solution. If the PMO sequence has been completed, wash the resin with IPA four times and dry the resin.

### Cleavage and Deprotection

Treat dry, detritylated PMO-bound resin with NMP and allow it to swell for 3 h at 30° C. Drain the resin, add cleavage cocktail containing 1.0-1.4 M DTT, 2.0-2.8 M DBU in NMP to the resin, and react at 40° C. for 2 h. Collect the filtrate into a clean filtration flask. Dilute the cleaved PMO solution with chilled ammonium hydroxide to form a uniform mixture. Place the PMO solution into a pressure flask and incubate at 50° C. for 18 h at 120 rpm. Repeat this process for a second round of cleavage as detailed above. Collect both cleavage solutions and dilute with water.

### PMO Purification

Concentrate the diluted PMO cleavage solution by tangential flow filtration (TFF). Perform diafiltration with water until the conductivity was <300 μS/cm. Concentrate desalted PMO to an adequate volume for anion exchange (AEX) purification. Load and purify crude PMO using an AEX column (TOYOPEARL SuperQ-650S) with the following gradient buffers: Buffer A=10-25 mM NaOH; Buffer B=10-25 mM NaOH+0.5-1 M NaCl. Pure fractions were identified by ion-pair reverse-phase chromatography (IP-RP) and pooled for desalting by TFF. IP-RP method was performed with a C18 column and the following gradient buffers: Buffer A=10 mM triethylamine (TEA), 4.3-8.6 mM Na₃PO₄, 10 mM dodecyltrimethylammonium bromide (DTMA) in 55% methanol; Buffer B=10 mM TEA, 4.3 Na₃PO₄, 10 mM DTMA in 60% acetonitrile. Perform diafiltration until the pure PMO solution has a conductivity <350 μS/cm. Concentrate and lyophilize the desalted pure PMO to yield a white powder. Determine purity of pure PMO by the above IP-RP method.

### Example 5: EEV-PMO Synthesis-Method 1

### Conjugation of EEV to PMO

Dissolve PMO in DMSO. Prepare separate solutions of EEV in DMSO, HATU in DMSO, and DIPEA in DMSO. Add DIPEA, HATU, and EEV solutions to the dissolved PMO solution. Analyze reaction progress by CEX or RP-HPLC.

### TFA Deprotection

For Generation 1 conditions, dilute the conjugation reaction with water and mix. Dilute the mixture with a solution containing 25 mM NaOH and 0.2 M KCl and mix. Analyze reaction progress by CEX or RP-HPLC. When the deprotection is complete, dilute the reaction with 0.5 M NaH₂PO₄ buffer before purification.

For Generation 2 conditions, dilute the mixture with a solution containing 320 mM NaOH and mix. Analyze the reaction progress by CEX or RP-HPLC. When the deprotection is complete, dilute the reaction with 0.5 MNaH₂PO₄ buffer before purification.

### EEV-PMO Purification

Concentrate the diluted PMO cleavage solution by tangential flow filtration (TFF). Concentrate desalted PMO to an adequate volume for cation exchange (CEX) purification. Load and purify crude PMO using an CEX column with the following gradient buffers: Buffer A=water, or 10-25 mM NaH₂PO₄ in 15-20% acetonitrile; Buffer B=0.5-1 M NaCl, or 10-25 mM NaH₂PO₄, 0.5-1 M NaCl in 15-20% acetonitrile. Pure fractions were identified by ion-pair reverse-phase chromatography (IP-RP) and pooled for desalting by TFF. Concentrate and lyophilize the desalted pure PMO to yield a white powder. Determine purity of pure PMO by CEX method, performed with a strong cation exchange (SCX) column and the following gradient buffers: Buffer A=24 mM H₃PO₄ in 80% water and 20% acetonitrile; Buffer B=24 mM H, 2.0 M LiCl in 75% water and 25% acetonitrile.

### Example 6: Optimization of Reaction Conditions

### Optimization of Linear Peptide Fragment Synthesis.

Fmoc-Proline is replaced by Ac-Proline and decreases the number of synthetic steps to prepare linear peptide fragment. In the generation 1 protocol, Fmoc-Proline is coupled as the last amino acid in the linear peptide sequence. An Fmoc deprotection is then performed and then the free amine of the Proline residue is protected by an acetyl group (via reaction with acetic anhydride). This protocol can be performed manually for larger scale reactions (>1 g) and in an automated fashion using a smaller scale (˜600 mg of 0.4 mmol/g loaded material per 40 mL reactor).

### Suppressing Epimerization During Dipeptide Coupling for Cyclic Peptide Synthesis:

Investigated alternative protecting groups instead of Fmoc for the dipeptide Gly-Arg (Pbf). Pht, Dde, and Fmoc/Dmb were investigated for use in cyclic peptide synthesis and epimerization was assessed throughout the synthetic process and in the final product

TABLE 5
Summary table of Pht deprotection screen
using hydrazine with and without Oxyma.

| | | Pro- | Depro- | Deprotected + | Open Chain |
|---|---|---|---|---|---|
| | Time | tected | tected | Hydrazine | Intermediate |
| Solution | (min) | (%) | (%) | (%) | (%) |
| 2% | 30 | 11.68 | 51.13 | 12.99 | 24.20 |
| hydrazine | 60 | 11.03 | 59.07 | 11.59 | 18.31 |
| | 90 | 9.92 | 56.27 | 10.88 | 22.93 |
| | 120 | 9.46 | 61.35 | 9.17 | 20.02 |
| 5% | 30 | 22.18 | 42.88 | 13.18 | 21.76 |
| hydrazine | 60 | 19.68 | 48.42 | 12.50 | 19.40 |
| | 90 | 17.62 | 49.36 | 10.84 | 22.19 |
| | 120 | 11.74 | 61.35 | 6.80 | 20.10 |
| 10% | 30 | 15.53 | 38.52 | 20.19 | 21.03 |
| hydrazine | 60 | 9.08 | 31.74 | 30.45 | 20.65 |
| | 90 | 6.29 | 28.42 | 32.66 | 24.47 |
| | 120 | 4.03 | 22.36 | 40.00 | 26.75 |
| 10% | 30 | 22.03 | 44.99 | 13.50 | 11.48 |
| hydrazine, | 60 | 17.25 | 44.26 | 19.10 | 12.13 |
| 1M | 90 | 9.18 | 48.45 | 25.77 | 9.93 |
| Oxyma | 120 | 7.23 | 40.93 | 29.45 | 12.46 |
| 15% | 30 | 6.09 | 24.09 | 35.35 | 23.25 |
| hydrazine | 60 | 1.94 | 9.60 | 54.13 | 25.65 |
| | 90 | 1.09 | 4.47 | 49.85 | 26.67 |
| | 120 | 0.29 | 2.29 | 57.02 | 30.76 |
| 15% | 30 | 17.36 | 36.92 | 21.43 | 13.92 |
| hydrazine, | 60 | 5.95 | 27.07 | 29.20 | 12.73 |
| 1M | 90 | 1.90 | 19.76 | 51.47 | 14.34 |
| Oxyma | 120 | 1.09 | 13.95 | 61.74 | 14.01 |

TABLE 6
Summary table of Pht deprotection screen using hydrazine
with allyl alcohol and methyl hydrazine.

| | | Pro- | Depro- | Deprotected + | Open Chain |
|---|---|---|---|---|---|
| | Time | tected | tected | Hydrazine | Intermediate |
| Solution | (min) | (%) | (%) | (%) | (%) |
| 5% | 30 | 23.47 | 40.12 | 14.13 | 19.57 |
| hydrazine, | 60 | 21.22 | 43.50 | 15.56 | 19.36 |
| 200 eq | 90 | 21.98 | 41.07 | 14.66 | 19.72 |
| allyl | 120 | 16.37 | 42.08 | 19.04 | 22.28 |
| alcohol | | | | | |
| 15% | 30 | 6.09 | 24.09 | 35.35 | 23.25 |
| hydrazine, | 60 | 1.94 | 9.60 | 54.13 | 25.65 |
| 200 eq | 90 | 1.09 | 4.47 | 49.85 | 26.67 |
| allyl | 120 | 0.29 | 2.29 | 57.02 | 30.76 |
| alcohol | | | | | |
| 5% methyl | 30 | 22.61 | 34.45 | 21.88 | 8.97 |
| hydrazine | 60 | 23.36 | 33.64 | 19.48 | 10.21 |
| | 90 | 22.94 | 35.91 | 22.34 | 7.68 |
| | 120 | 23.19 | 38.32 | 21.06 | 7.94 |
| 15% | 30 | 20.91 | 39.35 | 19.13 | 12.09 |
| methyl | 60 | 21.99 | 35.12 | 19.20 | 13.31 |
| hydrazine | 90 | 19.74 | 43.55 | 22.48 | 11.13 |
| | 120 | 22.68 | 37.29 | 19.95 | 9.49 |
| 15% | 30 | 11.54 | 30.40 | 21.46 | 18.30 |
| methyl | 60 | 12.30 | 31.31 | 23.14 | 18.14 |
| hydrazine | | | | | |
| (2x | | | | | |
| 30 min) | | | | | |

TABLE 7
Summary Table of Cyclic Peptide Fragment Purity Synthesized with
Various Gly Protecting Groups. (Purity by UPLC Integration)

| | Gen 1 | Cyclic | Cyclic 1 with | Cyclic 1 with |
|---|---|---|---|---|
| | Cyclic | 1 with | Dde (0.075 | Dde (0.322 |
| | Peptide | Dmb | mmol scale) | mmol scale) |
| Product Peak % | 84.69 | 89.99 | 74.41 | 85.26 |
| Epimer Peak % | 6.15 | 2.46 | 25.59 | 4.80 |
| Impurity Peak % | N/A | N/A | N/A | 2.92 |
| | | | | (Arg(Pbf) |
| | | | | addition) |
| Dimer Peak % | 9.14 | 7.54 | 4.04 | 7.02 |

### Dipeptide Coupling Screen to Suppress Epimerization During Synthesis of Cyclic Peptide

Alternative dipeptide coupling reagents/reaction conditions were assessed after adjusting protocol to include coupling of pre-made dipeptide (initially meant to limit DKP side product). Epimerization was assessed after the dipeptide coupling and optimized conditions were carried forward

TABLE 8
Dipeptide Coupling Screen during Cyclic Peptide
Synthesis (purity by UPLC integration)

| | Coupling | |
|---|---|---|
| Condition | Reagent/Additive | % Epimer |
| 1 | HOAt/DIC | 4% |
| 2 | HOAt/DIC* | 3% |
| 3 | Oxyma/DIC | 8% |
| 4 | DIPEA/DEPBT | 25% |
| 5 | NMM/DEPBT | 8% |
| 6 | K-Oxyma/DIC | 34% |
| 7 | Oxyma-B/DIC | 1% |
| 8 | NMM/HATU | 33% |

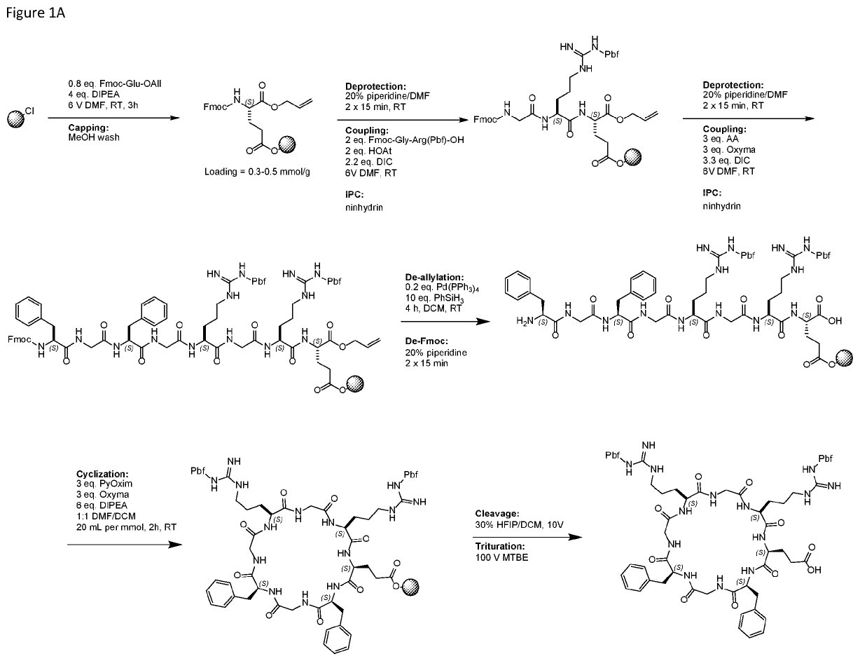

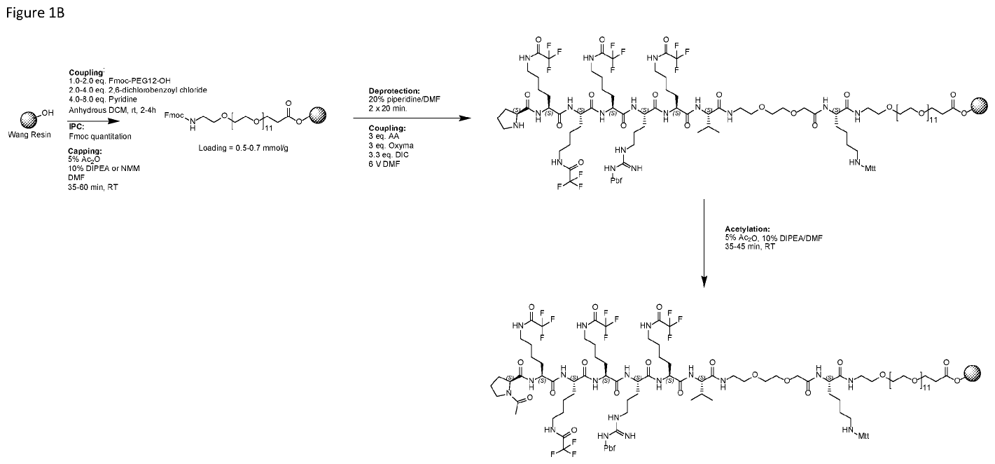

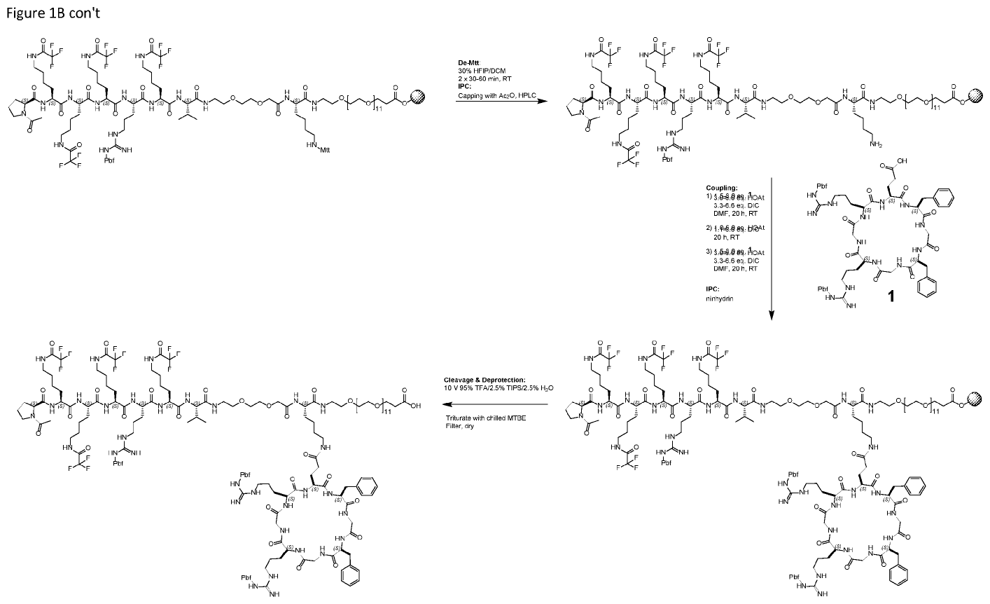

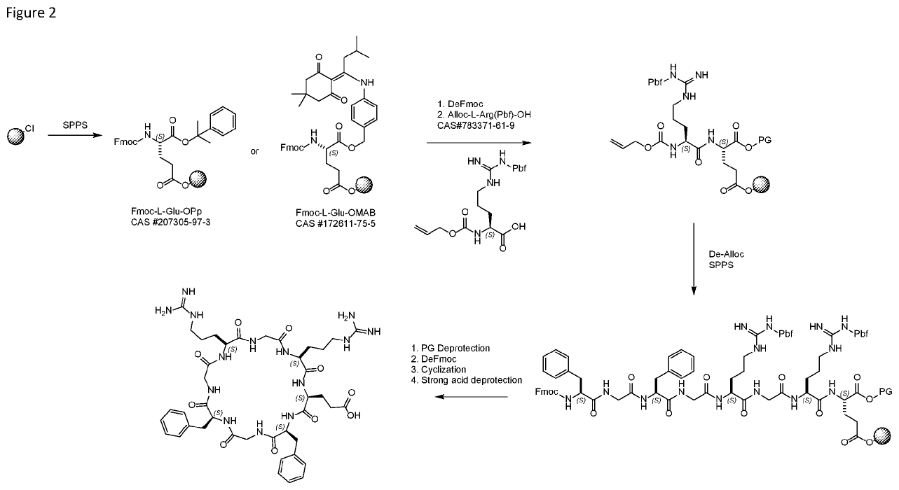

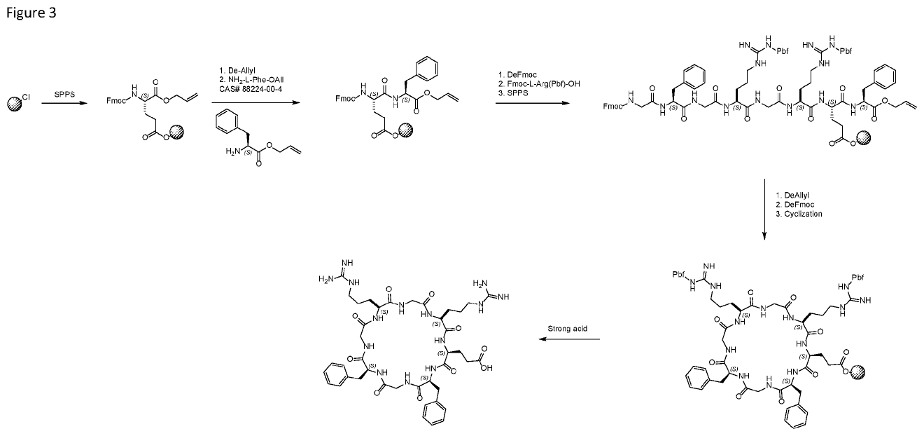

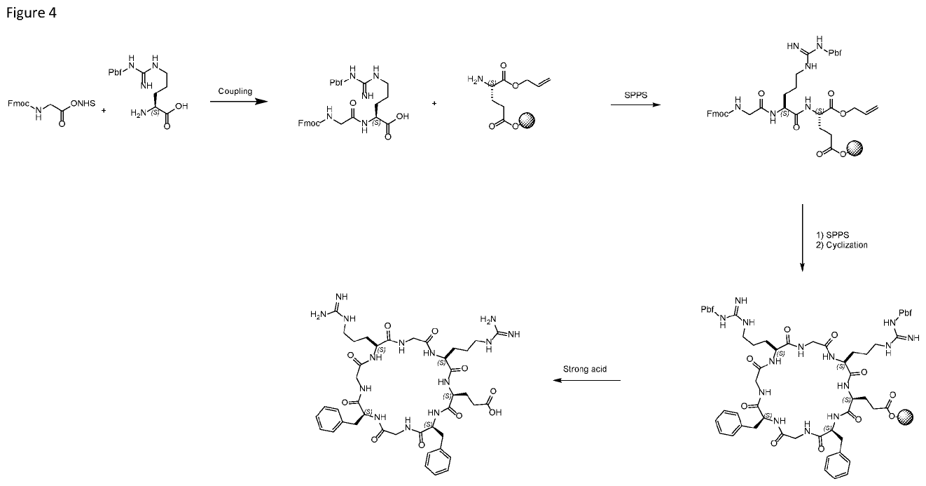

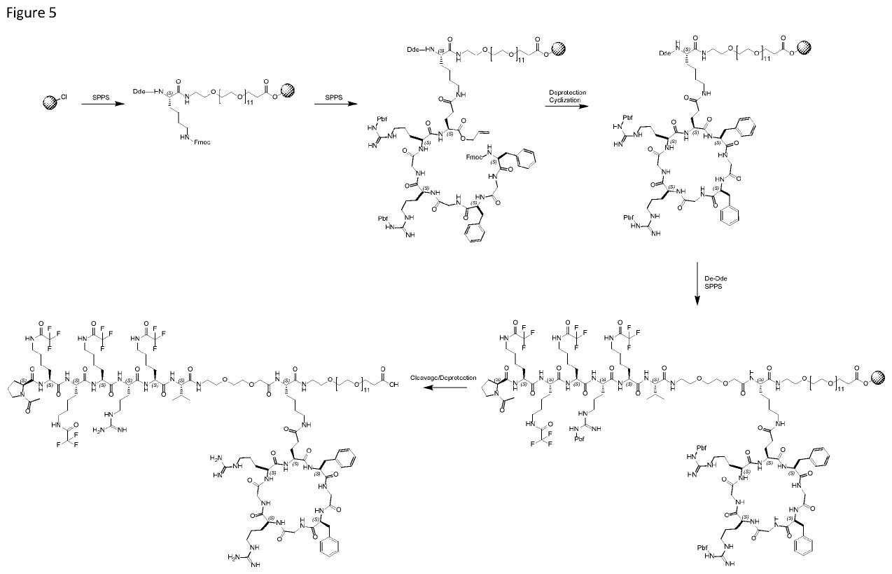

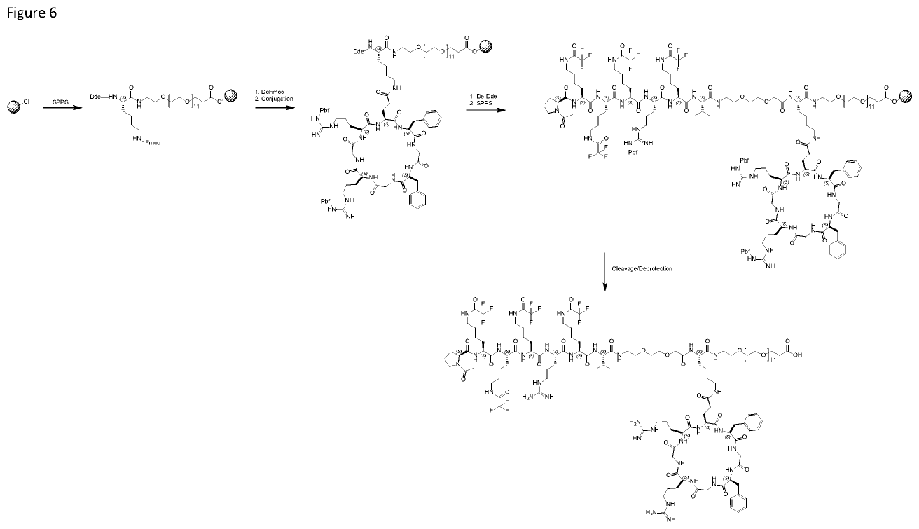

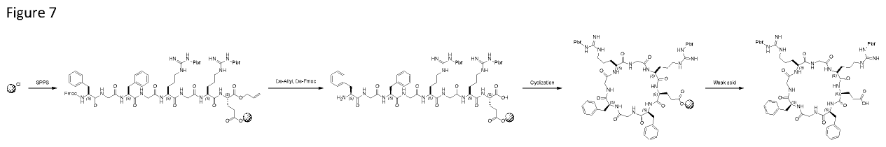

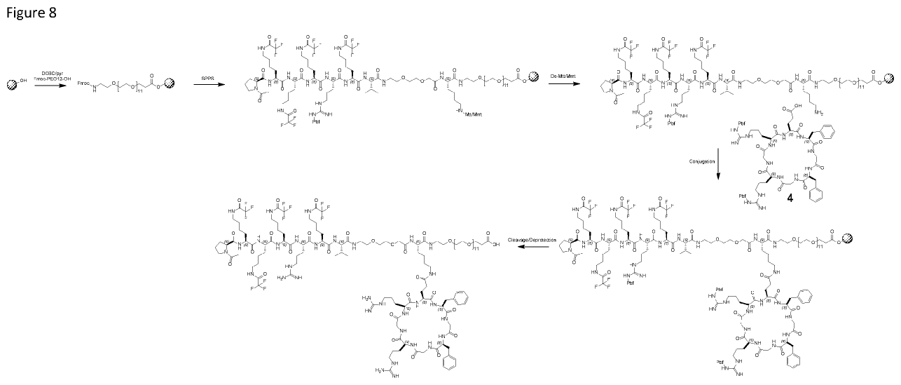

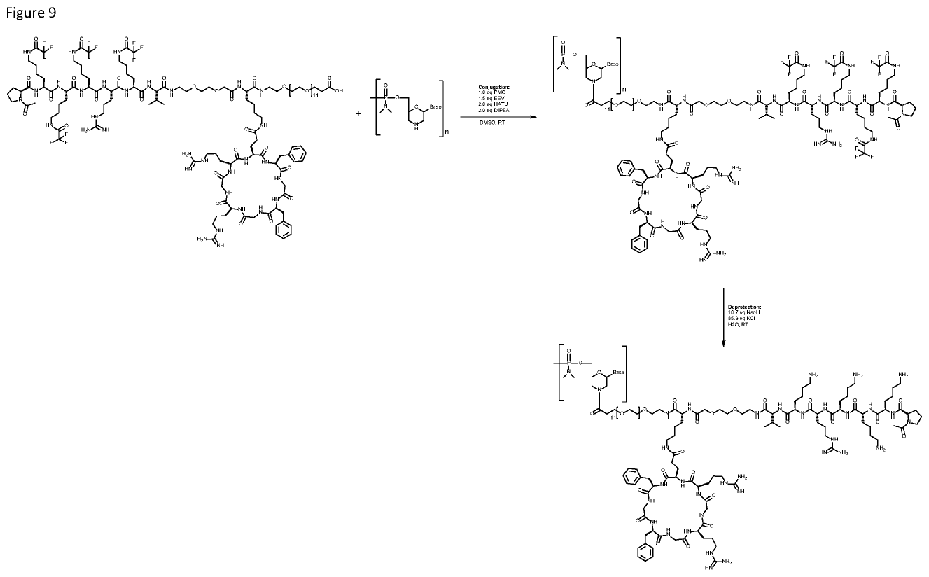

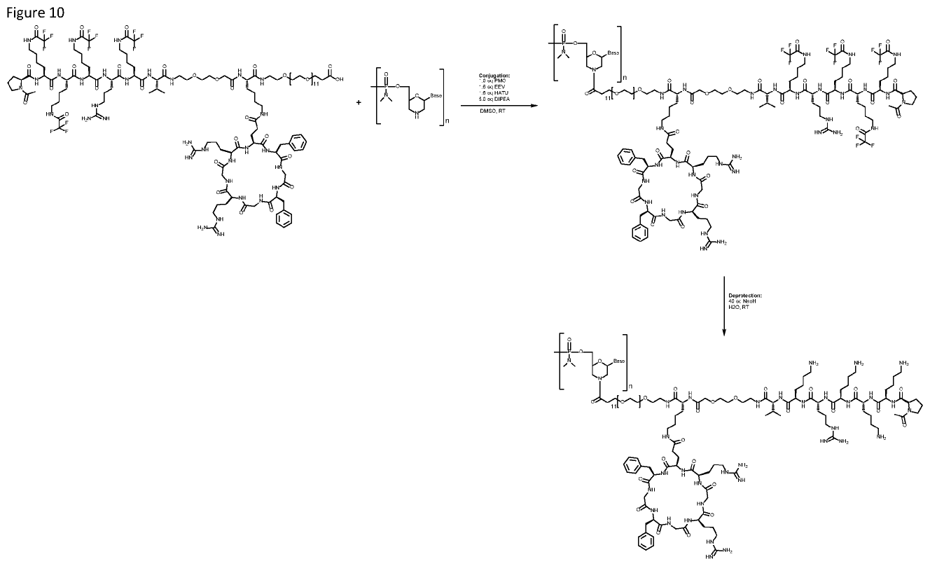

### Deprotection of Allyl Ester Optimization (FIG. 1a)

TABLE 9
Deprotection of allyl ester

| | | | Fmoc-R- |
|---|---|---|---|
| | | | E-Oallyl |
| | Desired | | Starting |
| Conditions | Product | Byproduct | Material |
| 20% piperidine, 5 min | 91.5 | 0.31 | 0 |
| 2% DBU, 5 min | 90.3 | 2.2 | 0 |
| 20% piperidine, 40 min | 79.4 | 12.8 | 0 |
| 2% DBU, 40 min | 88.7 | 4.9 | 0 |
| 2% DBU/5% piperidine/5% Oxyma, | 82.8 | 9.9 | 0 |
| 40 min | | | |
| 2% DBU/5% piperidine/0.5M | 82.9 | 9.4 | 0 |
| Oxyma, 40 min | | | |
| 2% DBU/5% piperidine/1.0M | 56.5 | 0 | 19.7 |
| Oxyma, 40 min | | | |
| 2% DBU/5% piperidine/1.0M HOBt, | 18.8 | 0.2 | 80.1 |
| 40 min | | | |
| 0.2M NaOh in 1:1 2-MeTHF:MeOH, | 0 | 0 | 0 |
| 40 min | | | |
| 2% DBU/5% piperazine/1% formic | 41.2 | 4.1 | 53.6 |
| acid, 40 min | | | |
| 0.15 TBAF in 1:1 DMF/MeOH, 40 | 3.9 | 0 | 93.9 |
| min | | | |
| 30% ter-butylamine in DMF, 40 min | 96.1 | 2.9 | 0 |
| 10% piperidine, 40 min | 76.5 | 21 | 0 |
| 20% piperidine, 40 min, cooling | 86.8 | 11.3 | 0 |
| 2% DBU, 40 min, cooling | 84.7 | 5.2 | 0 |
| 20% pyrrolidine/NMP, 40 min | 81.3 | 16.8 | 0 |
| 20% morpholine/DMF, 40 min | 78.8 | 3.3 | 16.4 |
| 20% diethylamine/DCM, 40 min | 79.4 | 1.6 | 11 |
| precooling 20% piperidine, 2 min, | 89.3 | 7.8 | 0 |
| twice | | | |
| precooling 2% DBU, 2 min, twice | 71.3 | 19.7 | 0 |

### Fmoc Deprotection Screen for Linear Peptide Synthesis

Alternative deprotection conditions discovered that can replace deprotection with 20% piperidine/DMF without affecting product integrity/purity. Milder conditions resulted in complete Fmoc removal, and impurities, such as desPeg₁₂ and desPeg₁₂Lys(Mtt) that typically result from harsh deprotections were observed

TABLE 10
Fmoc Deprotection

| | Average | Average | Average |
|---|---|---|---|
| | UV-vis | UV-vis | UV-vis |
| | Absorbance* | Absorbance* | Absorbance* |
| Deprotection Conditions | 1^{st} wash | 2^{nd} wash | 3^{rd} wash |
| 20% piperidine/DMF | 1.78 | 0.16 | N/A |
| 10% piperidine/DMF | 1.76 | 0.20 | N/A |
| 5% piperidine/DMF | 1.79 | 0.19 | N/A |
| 2% piperidine/DMF | 1.79 | 0.80 | 0.05 |
| 2% piperidine, 2% | 1.76 | 1.41 | 0.28 |
| DBU/DMF | | | |
| 20% piperidine, 1% | 1.79 | 1.60 | 0.49 |
| formic acid/DMF | | | |
| 20% piperidine, 1M | N/A | N/A | N/A |
| oxyma/DMF | | | |

*Average UV-Vis absorbance measurements at 304 nm for alternative deprotection conditions

The table above details alternative Fmoc deprotection conditions. The deprotection filtrates were analyzed by UV to quantitate removed Fmoc.

Additionally, LCMS was performed to assess integrity/quality of deprotected linear peptide products. 20%, 10%, 5%, and 2% piperidine in DMF each yielded similar results, demonstrating that a reduction of piperidine can still fully deprotect the Fmoc-protected peptide. Use of an organic base (DBU) and addition of an acid to tune basicity of piperidine were also performed. Based on UV and LCMS analysis, the integrity of the linear peptide products was comparable to the control, so these alternative methods can also be used for Fmoc deprotection. No deletion products were detected by LCMS.

### Cyclization Optimization (FIG. 1A)

TABLE 11
Cyclization conditions

| Condition | Cyclized (%) | Uncyclized (%) | Dimer (%) |
|---|---|---|---|
| HATU/HOAt/DIPEA | 84.2 | 0 | 15.8 |
| PyAOP/HOAt/DIPEA | 71.2 | 12.1 | 14.1 |
| PyAOP/HOAt/NMM | 87.1 | 0 | 14.6 |
| PyBOP/HOBt/DIPEA | 86.8 | 0 | 13.1 |
| PyOxim/Oxyma/DIPEA | 97 | 0 | 3 |
| DIC/HOBt/DIPEA | 74 | 0 | 25.9 |
| HBTU/HOBt/DIPEA | 76.4 | 0 | 23.5 |
| TBTU/HOBT/DIPEA | 75.1 | 0 | 24.9 |
| COMU/Oxyma/DIPEA | 94.3 | 0 | 5.6 |
| DEPBT/DIPEA | 86.8 | 0 | 13.2 |

Additional cyclization reagents were screened during synthesis of the cyclic peptide fragment. Cyclization screen was performed at two resin loading levels (high/low) to assess differences in product, epimer, and dimer formation.

TABLE 12
Cyclization conditions
Percent Purity by UPLC Integration of Cyclic
Peptide Peptide Fragment, 0.46 mmol/g

| | Cyclic | Epimer | Dimer | Uncyclized |
|---|---|---|---|---|
| | Peptide | (10.33 | (15.69 | Pdt (6.49 |
| Condition | (10.13 min) | min) | min) | min) |
| *PyOxim/Oxyma/DIPEA | 88.59 | 4.82 | 6.58 | — |
| HATU/HOAt/DIPEA | 84.53 | 4.92 | 10.53 | — |
| DEPBT/DIPEA | 83.92 | 5.37 | 10.69 | — |
| PyAOP/HOAt/DIPEA | 83.73 | 5.09 | 11.16 | — |
| PyBrOP/DIPEA | 67.22 | 6.20 | 2.11 | 24.45 |
| CDI/DIPEA | — | 2.24 | — | 91.08 |

*Control

TABLE 13
Cyclization conditions
Percent Purity by UPLC Integration of
Cyclic Peptide Fragment, 0.22 mmol/g

| | Cyclic | Epimer | Dimer | Uncyclized |
|---|---|---|---|---|
| | Peptide | (10.33 | (15.69 | Pdt (6.49 |
| Condition | (10.13 min) | min) | min) | min) |
| PyOxim/Oxyma/DIPEA* | 91.12 | 4.31 | 5.56 | — |
| DEPBT/DIPEA | 82.85 | 5.02 | 12.11 | — |
| HATU/HOAt/DIPEA | 82.37 | 4.46 | 13.15 | — |
| PyAOP/HOAt/DIPEA | 80.60 | 4.50 | 14.79 | — |
| CDI/DIPEA | — | 1.89 | — | 95.20 |

*Control

In most conditions, desired product formation and cyclization was complete with varying amounts of epimer and dimer side products. Unexpectedly, the lower resin loading level results in higher amounts of dimer formation in most cases, but lower amounts of epimer.

### Optimization to Reduce Dimerization During Cyclic Peptide Synthesis

A resin loading screen was performed to assess and minimize dimer formation during cyclization of the linear precursor and its reproducibility at a large scale.

TABLE 14
Summary table of resin loading levels, desired product, epimer,
and dimer detected after the cyclization of the linear precursor.
Percent Purity by UPLC Integration of Cyclic Peptide Fragment

| Resin Loading | Cyclic Peptide | Epimer | Dimer |
|---|---|---|---|
| (mmol/g) | (10.13 min) | (10.33 min) | (15.69 min) |
| 0.22 | 90.12 | 4.31 | 5.56 |
| 0.39 | 88.18 | 5.86 | 5.81 |
| 0.46 | 88.59 | 4.82 | 6.58 |
| 0.64 | 87.75 | 5.99 | 6.38 |
| 0.77 | 82.38 | 9.10 | 8.36 |
| 0.92 | 81.91 | 9.25 | 8.98 |
| Gen 1* | 84.69 | 6.15 | 9.14 |

*Gen 1 resin loading is 0.48-0.51 mmol/g

### Amide Conjugation Condition Screening

All conditions evaluated at 2 mM PMO (50 mg/mL in DMSO) at room temperature. All reagents prepared in DMSO:
1. DIC (300 mM DMSO)
2. Oxyma (300 mM DMSO)
3. PyAOP (300 mM DMSO)
4. HATU (300 mM DMSO)
5. DIPEA (300 mM DMSO)
6. EEV (100 mM DMSO)
   Diluted/quenched with 1:1 ACN/H₂O+0.1% TFA before injection

TABLE 15
Amide Conjugation Condition

| | Coupling | | | | | Conversion |
|---|---|---|---|---|---|---|
| EEV | Reagent | Additive | Base | Solvent | Time | (%) |
| 2 eq. | 2 eq. | 1.5 eq. | 2 eq. DIPEA | DMSO | 2 h | 67.46 |
| | HATU | Oxyma | | | | |
| 2 eq. | 3 eq. | 2.5 eq. | 2 eq. DIPEA | DMSO | 2 h | 68.85 |
| | HATU | Oxyma | | | | |
| 2 eq. | 4 eq. | 3.5 eq. | 2 eq. DIPEA | DMSO | 2 h | 67.55 |
| | HATU | Oxyma | | | | |
| 2 eq. | 5 eq. | 4.5 eq. | 2 eq. DIPEA | DMSO | 2 h | 67.73 |
| | HATU | Oxyma | | | | |
| 2 eq. | 2 eq. | 1.5 eq. | 4 eq. DIPEA | DMSO | 2 h | 68.23 |
| | HATU | Oxyma | | | | |
| 2 eq. | 3 eq. | 2.5 eq. | 4 eq. DIPEA | DMSO | 2 h | 69.85 |
| | HATU | Oxyma | | | | |
| 2 eq. | 4 eq. | 3.5 eq. | 4 eq. DIPEA | DMSO | 2 h | 68.75 |
| | HATU | Oxyma | | | | |
| 2 eq. | 5 eq. | 4.5 eq. | 4 eq. DIPEA | DMSO | 2 h | 77.04 |
| | HATU | Oxyma | | | | |
| 2 eq. | 2 eq. | N/A | 2 eq. DIPEA | DMSO | 2 h | 77.23 |
| | HATU | | | | | |
| 2 eq | 4 eq. | N/A | 2 eq. DIPEA | DMSO | 2 h | 81.87 |
| | HATU | | | | | |
| 2 eq. | 2 eq. | N/A | 4 eq. DIPEA | DMSO | 2 h | 78.37 |
| | HATU | | | | | |

### Tfa Deprotection Condition Screening

TABLE 16
EEV-PMO Tfa Deprotection

| Condition | Time | | Completion |
|---|---|---|---|
| 0.2M NaOH | 30 | min | + |
| 0.1M NaOH | 30 | min | + |
| 10% hydrazine | 30 | min | − |
| 50 mM NaHCO₃, pH 10 | 1 | h | − |
| 50 mM NaHCO₃, pH 11 | 1 | h | − |
| 0.2M KCl, pH 12 | 2 | h | + |
| 50 mM NH₃—NH₄Cl, pH | 1 | h | − |
| 10 | | | |
| 50 mM NH₃—NH₄Cl, pH | 1 | h | − |
| 11 | | | |
| 2% piperidine | 1 | h | − |
| 5% piperidine | 1 | h | − |
| 10% piperidine | 1 | h | − |
| 20% piperidine | 1 | h | − |
| 1M EDT | 1 | h | − |
| 1M BME | 1 | h | − |

* A (+) indicates full deprotection, a (−) indicates partial or no deprotection

TABLE 17
EEV-PMO Conjugation

| | Coupling | | | | | Conversion |
|---|---|---|---|---|---|---|
| EEV | Reagent | Additive | Base | Solvent | Time | (%) |
| 2 eq. | 2 eq. | 1.5 eq. | 2 eq. DIPEA | DMSO | 2 h | 36.20 |
| | PyAOP | Oxyma | | | | |
| 2 eq. | 3 eq. | 2.5 eq. | 2 eq. DIPEA | DMSO | 2 h | 37.85 |
| | PyAOP | Oxyma | | | | |
| 2 eq. | 4 eq. | 3.5 eq. | 2 eq. DIPEA | DMSO | 2 h | 36.44 |
| | PyAOP | Oxyma | | | | |
| 2 eq. | 5 eq. | 4.5 eq. | 2 eq. DIPEA | DMSO | 2 h | 33.04 |
| | PyAOP | Oxyma | | | | |
| 2 eq. | 2 eq. | 1.5 eq. | 4 eq. DIPEA | DMSO | 2 h | 40.11 |
| | PyAOP | Oxyma | | | | |
| 2 eq. | 3 eq. | 2.5 eq. | 4 eq. DIPEA | DMSO | 2 h | 41.06 |
| | PyAOP | Oxyma | | | | |
| 2 eq. | 4 eq. | 3.5 eq. | 4 eq. DIPEA | DMSO | 2 h | 38.03 |
| | PyAOP | Oxyma | | | | |
| 2 eq. | 5 eq. | 4.5 eq. | 4 eq. DIPEA | DMSO | 2 h | 30.59 |
| | PyAOP | Oxyma | | | | |
| 2 eq. | 2 eq. | N/A | 2 eq. DIPEA | DMSO | 2 h | 39.83 |
| | PyAOP | | | | | |
| 2 eq. | 3 eq. | N/A | 2 eq. DIPEA | DMSO | 2 h | 40.59 |
| | PyAOP | | | | | |
| 2 eq. | 4 eq. | N/A | 2 eq. DIPEA | DMSO | 2 h | 34.35 |
| | PyAOP | | | | | |
| 2 eq. | 5 eq. | N/A | 2 eq. DIPEA | DMSO | 2 h | 34.77 |
| | PyAOP | | | | | |
| 2 eq. | 2 eq. | N/A | 4 eq. DIPEA | DMSO | 2 h | 50.59 |
| | PyAOP | | | | | |
| 2 eq. | 3 eq. | N/A | 4 eq. DIPEA | DMSO | 2 h | 41.98 |
| | PyAOP | | | | | |
| 2 eq. | 4 eq. | N/A | 4 eq. DIPEA | DMSO | 2 h | 37.82 |
| | PyAOP | | | | | |
| 2 eq. | 5 eq. | N/A | 4 eq. DIPEA | DMSO | 2 h | 28.99 |
| | PyAOP | | | | | |

TABLE 18
EEV-PMO Conjugation

| | Coupling | | | | | Conversion |
|---|---|---|---|---|---|---|
| EEV | Reagent | Additive | Base | Solvent | Time | (%) |
| 1 eq. | 1 eq. DIC | 1 eq. | N/A | DMSO | 2 h | 17.46 |
| | | Oxyma | | | | |
| 2 eq. | 1 eq. DIC | 1 eq. | N/A | DMSO | 2 h | 16.75 |
| | | Oxyma | | | | |
| 3 eq. | 1 eq. DIC | 1 eq. | N/A | DMSO | 2 h | 19.27 |
| | | Oxyma | | | | |
| 5 eq. | 1 eq. DIC | 1 eq. | N/A | DMSO | 2 h | 25.06 |
| | | Oxyma | | | | |
| 1 eq. | 2 eq. DIC | 2 eq. | N/A | DMSO | 2 h | 21.42 |
| | | Oxyma | | | | |
| 2 eq. | 2 eq. DIC | 2 eq. | N/A | DMSO | 2 h | 10.00 |
| | | Oxyma | | | | |
| 3 eq. | 2 eq. DIC | 2 eq. | N/A | DMSO | 2 h | 21.47 |
| | | Oxyma | | | | |
| 5 eq. | 2 eq. DIC | 2 eq. | N/A | DMSO | 2 h | 29.69 |
| | | Oxyma | | | | |
| 1 eq. | 3 eq. DIC | 3 eq. | N/A | DMSO | 2 h | 8.19 |
| | | Oxyma | | | | |
| 2 eq. | 3 eq. DIC | 3 eq. | N/A | DMSO | 2 h | 11.75 |
| | | Oxyma | | | | |
| 3 eq. | 3 eq. DIC | 3 eq. | N/A | DMSO | 2 h | ~12 |
| | | Oxyma | | | | |
| 3 eq. | 3 eq. DIC | 3 eq. | N/A | DMSO | 24 h | 44.58 |
| | | Oxyma | | | | |
| 5 eq. | 3 eq. DIC | 3 eq. | N/A | DMSO | 2 h | 26.36 |
| | | Oxyma | | | | |
| 5 eq. | 3 eq. DIC | 3 eq. | N/A | DMSO | 24 h | 50.02 |
| | | Oxyma | | | | |
| 1 eq. | 5 eq. DIC | 5 eq. | N/A | DMSO | 2 h | 11.78 |
| | | Oxyma | | | | |
| 2 eq. | 5 eq. DIC | 5 eq. | N/A | DMSO | 2 h | 16.09 |
| | | Oxyma | | | | |
| 3 eq. | 5 eq. DIC | 5 eq. | N/A | DMSO | 2 h | 19.53 |
| | | Oxyma | | | | |
| 3 eq. | 5 eq. DIC | 5 eq. | N/A | DMSO | 24 h | 40.52 |
| | | Oxyma | | | | |
| 5 eq. | 5 eq. DIC | 5 eq. | N/A | DMSO | 2 h | 32.38 |
| | | Oxyma | | | | |
| 5 eq. | 5 eq. DIC | 5 eq. | N/A | DMSO | 24 h | 51.58 |
| | | Oxyma | | | | |

### EEV-PMO Tfa Deprotection Screen

Alternative EEV-PMO deprotection conditions were assessed with various aqueous, organic, and amphiphilic bases leading to mild deprotection conditions with less PMO regeneration and higher product purity as well as more aggressive conditions to perform a faster deprotection without sacrificing purity are also being screened

TABLE 19
Tfa Deprotection Screening Conditions

| | | | PMO | |
|---|---|---|---|---|
| | | | Regeneration | |
| | Tfa Time of | | at time of | PMO |
| | Completion | | Completion | Regeneration |
| Condition | (hr) | | (hr) | at 24 h |
| 1M K₂CO₃ in H₂O | 5-24 | hr | 1.75% | 1.75% |
| 1M K₂CO₃, 18 eq. NaOH | 2 | hr | 1.90% | 3.11% |
| in H₂O | | | | |
| 1M K₂CO₃, 20 eq. NaOH | 1 | hr | 1.84-2.08% | 3.79-4.18% |
| 1M NaOMe in MeOH | N/A | | N/A | 7.92-30.52% |
| 1:1 Piperidine:H₂O | N/A | | N/A | 2.44% |
| 1.5M K₂CO₃ in H₂O | 5-24 | hr | 2.02% | 2.02% |
| 10 eq. NaOH, 0.1M KCl in | N/A | | N/A | N/A |
| H₂O | | | | |
| 12.5 mM NaOH, 0.1M KCl | 5-24 | hr | 1.39-1.81% | 1.39-1.81% |
| in H₂O | | | | |
| 125 eq. K₂CO₃ in H₂O | 5-24 | hr | 2.62% | 2.62% |
| 125 eq. K₂CO₃ in H₂O, 10 | 5-24 | hr | 3.24% | 3.24% |
| eq. NaOH | | | | |
| 125 eq. K₂CO₃ in H₂O, 15 | 5 | hr | 2.53% | 3.19% |
| eq. NaOH | | | | |
| 125 eq. K₂CO₃ in H₂O, 20 | 3 | hr | 2.37% | 4.01% |
| eq. NaOH | | | | |
| 15 eq. LiOH in H₂O | N/A | | N/A | 1.40% |
| 15 eq. NaOH in H₂O | 5-24+ | hr | 1.8-1.86% | 1.8-1.86% |
| 15 eq. NaOH, 0.1M KCl in | N/A | | N/A | N/A |
| H₂O | | | | |
| 15 eq. NaOH, 0.1M LiCl in | N/A | | N/A | N/A |
| H₂O | | | | |
| 15 eq. NaOH, 0.1M NaCl | N/A | | N/A | N/A |
| in H₂O | | | | |
| 16 eq. NaOH in H₂O | 5-24 | hr | 2.47% | 2.47% |
| 17 eq. NaOH in H₂O | 5-24 | hr | 2.59% | 2.59% |
| 18 eq. NaOH in H₂O | 5-24 | hr | 1.69-2.56% | 1.69-2.56% |
| 18.75 mM NaOH, 0.1M | 1 | hr | 3.22% | 4.96% |
| KCl in H₂O | | | | |
| 188 eq. K₂CO₃ in H₂O | 5-24 | hr | 2.25% | 2.25% |
| 2M K₂CO₃ in H₂O | 5-24 | hr | 1.93% | 1.93% |
| 2:1 Piperidine:H₂O | N/A | | N/A | 2.32% |
| 20 eq. KOH in H₂O | 5-24 | hr | 1.87% | 1.87% |
| 20 eq. NaOH in H₂O | 4 | hr | 3.05% | N/A |
| 20 eq. NaOH, 0.1M KCl in | 2 | hr | 1.88% | 2.39% |
| H₂O | | | | |
| 22.5% NH₄OH in H₂O | 5-24 | hr | 2.01% | 2.01% |
| 22.5 eq. NaOH in H₂O | 3 | hr | 1.85% | 2.21% |
| 25 eq. KOH in H₂O | 2 | hr | 1.97% | 6.29% |
| 25 eq. NaOH in H₂O | 1 | hr | 2.65% | 6.30% |
| 250 eq. K₂CO₃ in H₂O H₂O | 5-24 | hr | 2.19% | 2.19% |
| 27.5 eq. NaOH in H₂O | 1 | hr | 1.99% | 5.05% |
| 30 eq. LiOH in H₂O | 2 | hr | 2.42% | 9.10% |
| 30 eq. NaOH in H₂O | 1-2 | hr | 2.27-2.85% | 11.14-11.67% |
| 30 eq. NaOH, 0.1M KCl in | 1 | hr | 1.97% | 5.79% |
| H₂O H₂O | | | | |
| 40 eq. K₂CO₃ in H₂O | 5-24 | hr | 1.60-2.91% | 1.60-2.91% |
| 40 eq. K₂CO₃ in H₂O, 15 | 4 | hr | 2.03% | 3.84% |
| eq. NaOH in H₂O | | | | |
| 40 eq. KOH in H₂O | 1 | hr | 2.77% | 9.82% |
| 40 eq. KOH, 0.1M KCl in | 1 | hr | 2.70% | 10.87% |
| H₂O | | | | |
| 40 eq. NaOH in H₂O | 1 | hr | 1.00 | 14.36% |
| 40 eq. NaOH in H₂O, 0.1M | 1 | hr | 2.79% | 14.49% |
| KCl in H₂O | | | | |
| 40 eq. NaOH in H₂O, 0.1M | 1 | hr | 3.54% | 22.57% |
| NaCl in H₂O | | | | |
| 40 eq. NaOH in H₂O, 0.1M | 1 | hr | 2.56% | 17.93% |
| LiCl in H₂O | | | | |
| 40 eq. Proton Sponge in | N/A | | N/A | N/A |
| ACN | | | | |
| 40 eq. Proton Sponge in | N/A | | N/A | N/A |
| DMF | | | | |
| 40 eq. Proton Sponge in | N/A | | N/A | N/A |
| DMSO | | | | |
| 80 eq. K₂CO₃ in H₂O | 5-24 | hr | 1.82-2.13% | 1.82-2.13% |
| 80 eq. K₂CO₃ in H₂O, 10 | 4 | hr | 1.97% | 3.16% |
| eq. NaOH in H₂O | | | | |
| 80 eq. K₂CO₃ in H₂O, 15 | 3 | hr | 1.94% | 3.57% |

eq. NaOH in H₂O

TABLE 20
Additions Deprotection Conditions

| | | | PMO | |
|---|---|---|---|---|
| | Tfa Time | | Regeneration | |
| | of | | at time of | PMO |
| | Completion | | Completion | Regeneration |
| Condition | (hr) | | (hr) | at 24 h |
| 22.5% NH₃ in H₂O | 5-24 | hr | 2.0% | 2.0% |
| 20 eq. KOH | 5-24 | hr | 1.9% | 1.9% |
| 18 eq. NaOH | 5-24 | hr | 1.7% | 1.7% |
| 40 eq. K₂CO₃ | 5-24 | hr | 1.6-2.9% | 1.6-2.9% |
| 80 eq. K₂CO₃ | 5-24 | hr | 2.1% | 2.1% |
| 1M K₂CO₃ | 5-24 | hr | 1.8-2.6% | 1.8-2.6% |
| 1.5M K₂CO₃ | 5-24 | hr | 2.0-2.3% | 2.0-2.3% |
| 2M K₂CO₃ | 5-24 | hr | 1.9-2.2% | 1.9-2.2% |
| 1M K₂CO₃ + 20 eq. NaOH | 1 | hr | 1.8% | 3.8-4.2% |
| (3.60 mM EEV-PMO) | | | | |
| 125 eq. K₂CO₃ + 18 eq. | 2 | hr | 1.9% | 3.1% |
| NaOH (4.64 mM EEV-PMO) | | | | |
| 125 eq. K₂CO₃ + 15 eq | 5 | hr | 2.5% | 3.2% |
| NaOH (4.64 mM EEV-PMO) | | | | |
| 80 eq. K₂CO₃ + 15 eq | 4-5 | hr | 2.2-2.5% | 3.6% |
| NaOH (4.64 mM EEV-PMO) | | | | |

### Linear-Cyclic Peptide Coupling Reaction Screen

A screen was performed using various conditions for the linear-cyclic coupling. The experiments were carried out at 0.6 mmol/g loaded material and at 0.4 mmol/g.

TABLE 21
Summary of conjugation reaction conditions with 0.6 mmol/g loaded linear peptide fragment
Conjugation Completion by Percent Purity from UPLC Integration (0.6 mmol/g loading)

| | | | | Starting | Desired |
|---|---|---|---|---|---|
| | | | | Material | Product |
| | | | Volume | (20.9 | (21.50 |
| Day 1 (20 h) | Day 2 (40 h) | Day 3 (60 h) | (mL) | min) | min) |
| 6 eq cyclic, no HOAt | — | — | 10 | 85.12 | 2.99 |
| 6 eq cyclic, 0.6 eq | — | — | 10 | 80.16 | 4.08 |
| DMAP | | | | | |
| 3 eq recycled cyclic | +3 eq HOAt, | — | 10 | 35.16 | 50.20 |
| (weight) | 3.3 eq DIC | | | | |
| 6 eq cyclic, 6.0 eq | — | — | 10 | 15.99 | 64.83 |
| Oxyma | | | | | |
| 3 eq cyclic | — | — | 10 | 33.25 | 66.48 |
| 3 eq recycled cyclic | +3 eq HOAt, | — | 10 | 17.48 | 71.58 |
| (purity) | 3.3 eq DIC | | | | |
| 3 eq cyclic | — | — | 5 | 19.70 | 75.04 |
| 3 eq cyclic | +3.3 eq DIC | — | 10 | 14.68 | 83.14 |
| 6 eq cyclic | +3 eq HOAt, | — | 10 | 9.02 | 88.62 |
| | 3.3 eq DIC | | | | |
| 3 eq cyclic | +1 eq cyclic, 1 eq | — | 10 | 9.34 | 88.84 |
| | HOAt, 1.1 eq | | | | |
| | DIC | | | | |
| 6 eq cyclic | — | — | 10 | 7.70 | 90.25 |
| 6 eq cyclic | — | — | 10 | 7.70 | 90.25 |
| 3 eq cyclic (weight) | +3 eq cyclic, | — | 10 | 2.20 | 96.40 |
| | 3 eq HOAt, | | | | |
| | 3.3 eq DIC | | | | |
| 3 eq cyclic | +3 eq HOAt, | +3 eq cyclic | 10 | 0.86 | 99.02 |
| | 3.3 eq DIC | | | | |
| 3 eq cyclic | +3 eq HOAt, | +3 eq cyclic, 3 eq | 10 | 0.41 | 99.14 |
| | 3.3 eq DIC | HOAt, 3.3 eq DIC | | | |

TABLE 22
Summary of conjugation reaction conditions with 0.4 mmol/g loaded linear peptide fragment
Conjugation Completion by Percent Purity from UPLC Integration (0.4 mmol/g loading)

| | | | | Starting | Desired |
|---|---|---|---|---|---|
| | | | | Material | Product |
| | | | Volume | (20.9 | (21.50 |
| Day 1 (20 h) | Day 2 (40 h) | Day 3 (60 h) | (mL) | min) | min) |
| 3 eq cyclic (weight) | — | — | 10 | 21.20 | 76.30 |
| 3 eq cyclic (purity) | — | — | 10 | 26.40 | 71.00 |
| 3 eq cyclic | +3 eq HOAt, | +3 eq cyclic, 3 eq | 10 | 0.29 | 99.71 |
| | 3.3 eq DIC | HOAt, 3.3 eq DIC | | | |
| 3 eq cyclic | +3 eq HOAt, | +3 eq recycled | 10 | 0.00 | 100.00 |
| | 3.3 eq DIC | cyclic, 3 eq HOAt, | | | |
| | | 3.3 eq DIC | | | |
| 3 eq cyclic | +3 eq HOAt, | +3 eq HOAt, 3.3 eq | 10 | 0.00 | 100.00 |
| | 3.3 eq DIC | DIC | | | |
| 3 eq cyclic, 6 eq HOAt, | Continued from | — | 10 | 0.00 | 100.00 |
| 6.6 eq DIC | Day 1 | | | | |

TABLE 23
Analysis of 2-day conjugation with
lower equivalents of cyclic peptide by weight.
Percent Purity by UPLC Integration

| Equivalents | | Starting | |
|---|---|---|---|
| by Weight | Hours | Material | Desired Product |
| 3.0 eq | 20 | 1.35 | 94.02 |
| — | 40 | 0.00 | 100.00 |
| 2.0 eq | 20 | 9.01 | 90.99 |
| — | 40 | 0.00 | 100.00 |
| 1.0 eq | 20 | 37.74 | 62.26 |
| — | 40 | 26.61 | 73.39 |
| 0.5 eq | 20 | 64.40 | 35.60 |
| — | 40 | 54.73 | 45.27 |

TABLE 24
Analysis of 2-day conjugation with
lower equivalents of cyclic peptide by purity
Percent Purity by UPLC Integration

| Equivalents | | Starting | |
|---|---|---|---|
| by Purity | Hours | Material | Desired Product |
| 3.0 eq | 20 | 0.00 | 100.00 |
| — | 40 | 0.00 | 100.00 |
| 2.0 eq | 20 | 11.33 | 88.67 |
| — | 40 | 0.00 | 100.00 |
| 1.0 eq | 20 | 22.70 | 77.30 |
| — | 40 | 17.72 | 82.28 |
| 0.5 eq | 20 | 55.59 | 44.41 |
| — | 40 | 45.42 | 54.48 |

### Mechanistic Investigation of EEV-PMO Conjugation and Deprotection Reactions

EEV-PMO conjugation/deprotection reactions were assessed via time course comparing Gen 2 and Gen 1 protocols. Conjugation/deprotection reactions with PMO of different purities and deprotection of purified, protected EEV-PMO were also studied.

TABLE 25
Product formation of EEV-PMO based on UPLC integration data
Conjugation Reaction Time Course of Product Formation
with PMO of Varying Purity

| Conjugation | 96.0% | 77.43% | 72.72% | 68.97% | 59.15% | 42.39% |
|---|---|---|---|---|---|---|
| Timepoint (min) | PMO | PMO | PMO | PMO | PMO | PMO |
| 0 | 0 | 0 | 0 | 0 | 0 | 0 |
| 30 | 93.29 | 79.31 | 83.94 | 70.76 | 58.32 | 40.91 |
| 60 | 90.7 | 87.18 | 86.24 | 74.15 | 58.65 | 43.09 |
| 90 | 90.9 | 83.5 | 81.25 | 75.12 | 58.87 | 44.97 |
| 120 | 90.65 | 82.79 | 82.06 | 73.82 | 59.29 | 44.06 |
| 150 | 90.82 | 84.18 | 82.22 | 73.9 | 59.75 | 44.43 |
| 180 | 90.52 | 83.62 | 82.89 | 75.34 | 59.2 | 45.68 |

TABLE 26
PMO consumption during conjugation based on UPLC
integration data (over same time course as Table 25)
Conjugation Reaction Time Course of PMO
Consumption with PMO of Varying Purity

| Conjugation | 96.0% | 77.43% | 72.72% | 68.97% | 59.15% | 42.39% |
|---|---|---|---|---|---|---|
| Timepoint (min) | PMO | PMO | PMO | PMO | PMO | PMO |
| 0 | 96.49 | 91.82 | 83.17 | 80.02 | 90.1 | 98.79 |
| 30 | 2.21 | 10.8 | 2.75 | 5.95 | 16.21 | 22.85 |
| 60 | 1.83 | 6.74 | 2.68 | 4.69 | 12.64 | 22.76 |
| 90 | 1.79 | 5.02 | 3.7 | 4.65 | 10.86 | 22.53 |
| 120 | 1.75 | 9 | 3.3 | 5.95 | 13.08 | 22.54 |
| 150 | 1.77 | 9.36 | 3.59 | 4.86 | 13.77 | 22.38 |
| 180 | 2.05 | 5.47 | 3.77 | 4.79 | 13.76 | 22.64 |

TABLE 27
Deprotection Reaction Time Course of Product Formation
with PMO of Varying Purity

| Deprotection | | | | | | |
|---|---|---|---|---|---|---|
| Timepoint | 96.0% | 77.43% | 72.72% | 68.97% | 59.15% | 42.39% |
| (min) | PMO | PMO | PMO | PMO | PMO | PMO |
| 0 | 80.88 | 81.71 | 71.57 | 64.42 | 62.68 | 23.01 |
| 30 | 85.34 | 82.49 | 85.89 | 77.71 | 64.73 | 52.13 |
| 60 | 85.02 | 79.5 | 86.58 | 79.5 | 62 | 51.48 |
| 90 | 82.64 | 77.07 | 84.78 | 80.4 | 60.15 | 53.16 |
| 120 | 83.1 | 80.5 | 79.86 | 76.59 | 62.06 | 51.44 |
| 150 | 80.85 | 80.13 | 80.55 | 75.45 | 64.05 | 50.96 |
| 180 | 79.24 | 76.67 | 75.04 | 72.42 | 63.07 | 53.03 |
| 240 | 79.16 | 75.09 | 69.94 | 68.7 | 62.2 | 50.55 |
| 300 | 77.73 | 73.8 | 72.39 | 69.04 | 60.22 | 50.27 |
| 1440 | 69.76 | 65.25 | 59.78 | 57.15 | 55.38 | 45.64 |

TABLE 28
Deprotection Reaction Time Course of PMO Regeneration
with PMO of Varying Purity

| Deprotection | | | | | | |
|---|---|---|---|---|---|---|
| Timepoint | 96.0% | 77.43% | 72.72% | 68.97% | 59.15% | 42.39% |
| (min) | PMO | PMO | PMO | PMO | PMO | PMO |
| 0 | 2.49 | 1.23 | 3.21 | 5.95 | 9.66 | 28.43 |
| 30 | 3.51 | 5.81 | 3.35 | 6.78 | 11.26 | 26 |
| 60 | 3.8 | 6.43 | 3.4 | 7.13 | 17.94 | 26.14 |
| 90 | 3.75 | 10.45 | 4.75 | 7.79 | 10.27 | 24.34 |
| 120 | 4.41 | 7.85 | 5.4 | 8.16 | 13.29 | 26.47 |
| 150 | 4.95 | 6.28 | 4.65 | 8.84 | 15.37 | 26.95 |
| 180 | 5.95 | 11.37 | 5.07 | 9.6 | 17.04 | 24.73 |
| 240 | 6.18 | 8.2 | 5.73 | 9.29 | 16.02 | 28.15 |
| 300 | 6.02 | 8.85 | 5.29 | 9.47 | 17.09 | 28.36 |
| 1440 | 19.16 | 25.64 | 27.66 | 31.83 | 30.4 | 56.77 |

TABLE 29
Conjugation Reaction Time Course of Product
Formation with Gen 1 and Gen 2 Conditions

| Conjugation | Process Chem | Process Chem | CMC | CMC |
|---|---|---|---|---|
| Timepoint (min) | (Gen 2) | (Gen 2) | (Gen 1) | (Gen 1) |
| 0 | 0 | 0 | 0 | 0 |
| 30 | 93.29 | 85.31 | 92.16 | 91.15 |
| 60 | 90.7 | 86.37 | 92.22 | 93.45 |
| 90 | 90.9 | 87.47 | 90.28 | 92.67 |
| 120 | 90.65 | 88.92 | 88.44 | 91.59 |
| 150 | 90.82 | 90.01 | 89.29 | 90.8 |
| 180 | 90.52 | 89.91 | 89.55 | 91.96 |

TABLE 30
Conjugation Reaction Time Course of PMO Consumption
with Gen 1 and Gen 2 Conditions

| | Process | Process | | |
|---|---|---|---|---|
| Conjugation | Chem | Chem | CMC | CMC |
| Timepoint (min) | (Gen 2) | (Gen 2) | (Gen 1) | (Gen 1) |
| 0 | 96.49 | 96.38 | 92.58 | 95.81 |
| 30 | 2.21 | 2.43 | 4.8 | 2.29 |
| 60 | 1.83 | 2.92 | 2.23 | 0.9 |
| 90 | 1.79 | 0.84 | 2.59 | 0.89 |
| 120 | 1.75 | 0.98 | 2.95 | 2.35 |
| 150 | 1.77 | 2.62 | 2.06 | 2.79 |
| 180 | 2.05 | 2.14 | 2.89 | 2.43 |

TABLE 31
Deprotection Reaction Time Course of Product
Formation with Gen 1 and Gen 2 Conditions

| | Process | Process Chem | | |
|---|---|---|---|---|
| Deprotection | Chem | (Gen 2) | CMC | Purified |
| Timepoint | (Gen 2) | Conj/CMC | (Gen 1) | Product |
| (min) | Conj/Depro | (Gen 1) Depro | Conj/Depro | Deprotection |
| 0 | 58.68 | 2.18 | 1.45 | 61.05 |
| 30 | 82.78 | 22.89 | 9.11 | 92.01 |
| 60 | 90 | 28.57 | 16.42 | 94.01 |
| 90 | 91.16 | 44.46 | 30.16 | 92.52 |
| 120 | 86.88 | 47.87 | 32.3 | 92.4 |
| 150 | 84.8 | 61.11 | 44.51 | 89.79 |
| 180 | 86.15 | 63.8 | 50.4 | 86.72 |
| 240 | 80.08 | 69.71 | 55.57 | 89.02 |
| 300 | 81.67 | 75.35 | 59.8 | 82.46 |
| 1440 | 72.86 | 89.81 | 89.47 | 57.92 |

TABLE 32
Deprotection Reaction Time Course of PMO Regeneration
with Gen 1 and Gen 2 Conditions

| | Process | Process Chem | | |
|---|---|---|---|---|
| Deprotection | Chem | (Gen 2) | CMC | Purified |
| Timepoint | (Gen 2) | Conj/CMC | (Gen 1) | Product |
| (min) | Conj/Depro | (Gen 1) Depro | Conj/Depro | Deprotection |
| 0 | 1.86 | 3.58 | 2.21 | 0 |
| 30 | 3.04 | 3.92 | 2.26 | 1.68 |
| 60 | 2.1 | 3.65 | 2.6 | 0.65 |
| 90 | 3.07 | 3.58 | 2.44 | 1.38 |
| 120 | 3.54 | 3.56 | 2.41 | 1.12 |
| 150 | 3.59 | 3.69 | 2.4 | 1.89 |
| 180 | 3.51 | 3.76 | 2.38 | 5.08 |
| 240 | 4.43 | 3.55 | 2.33 | 8.86 |
| 300 | 4.3 | 3.83 | 2.32 | 8.35 |
| 1440 | 18.87 | 3.78 | 2.75 | 10.36 |

Experiments were performed to investigate Gen 2 conjugation/deprotection protocols (40 eq NaOH), Gen 1 conjugation/deprotection protocols (conjugation stoichiometry is slightly varied, and deprotection uses 12.5 mM NaOH, 1M KCl), deprotection of purified, protected EEV-PMO with Gen 2 methods, and Gen 2 conjugation/deprotection with PMOs of various purities (4 levels of purity). In all of these experiments, both conjugation and deprotection reactions were monitored and compared using purity by UPLC integration and LCMS mass analysis.

1-45. (canceled)

46. A method of making an endosomal escape vehicle (EEV) comprising a cyclic cell penetrating peptide (cCPP), and a linear peptide comprising a linker and an exocyclic peptide (EP), wherein:
(a) the cCPP comprises Formula (I): 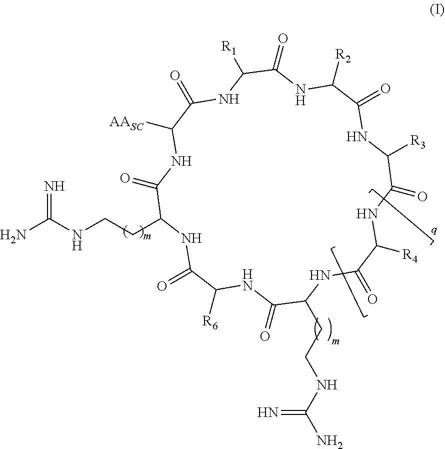
or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3;
(b) the linker comprises: 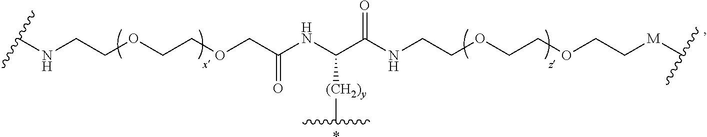
wherein:
x′ is an integer from 1-23; y is an integer from 1-5;
z′ is 11;
* is the point of attachment to the AA_{SC} of the cCPP; and
M is a bonding group; and
(c) the EP comprises from 4-8 amino acids, wherein the EP comprises at least one lysine residue and/or at least one arginine residue;
the method comprising:
(i) making a cyclic cell penetrating peptide (cCPP) of formula (VIII): 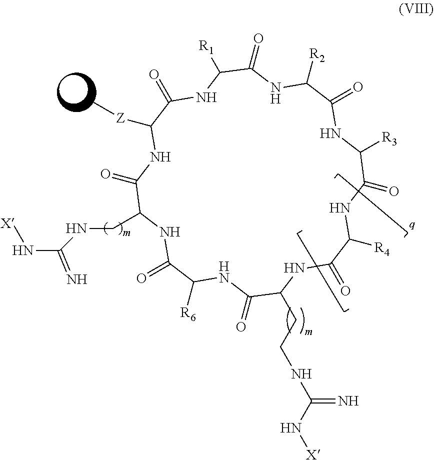
or a protonated form thereof,
wherein:
X′ is a protecting group and 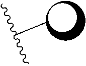
is a solid support and the method of making the cCPP of formula (VIII) comprises:
reacting a compound of formula (IV) 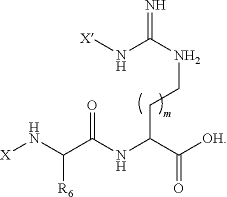
with a compound of formula (V) 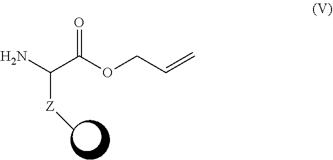
to form a compound of formula (VI) 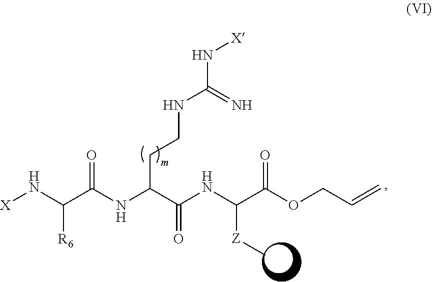
wherein Z is a radical of an amino acid side chain and X is a protecting group;
coupling the compound of formula (VI) with Fmoc protected amino acids (Fmoc-AA) by solid phase peptide synthesis to form a compound of formula (VII) 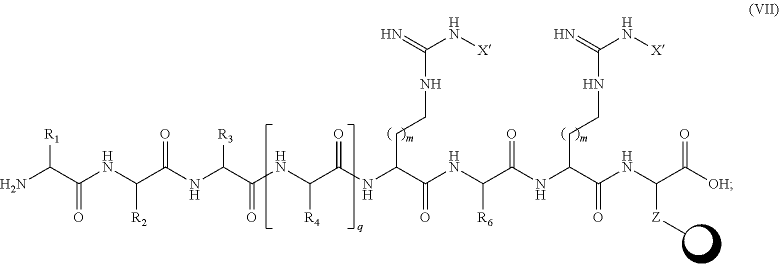
and
treating the compound of formula (VII) with PyOxim/Oxyma and a base to obtain a compound of formula (VIII): 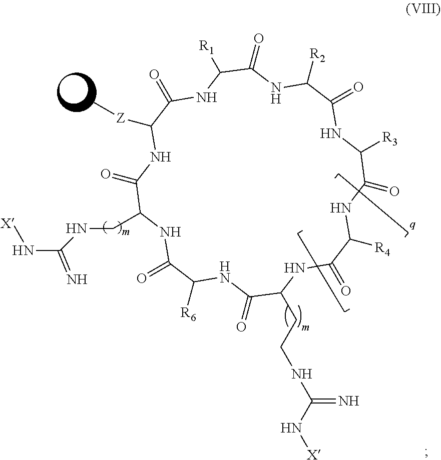
(ii) making the linear peptide, the method comprising:
coupling Fmoc-PEG₁₂-CH₂CH₂COOH onto a Wang resin to obtain: 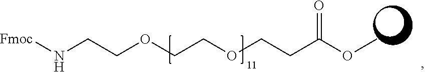
wherein 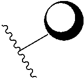
represents the resin;
removing the Fmoc protecting group to give a reaction product; and
coupling the reaction product with Fmoc protected amino acids (Fmoc-AA) by solid phase peptide synthesis to obtain the linear peptide; and
(iii) coupling the cCPP to the linear peptide.

47. The method of claim 46, wherein M is selected from: 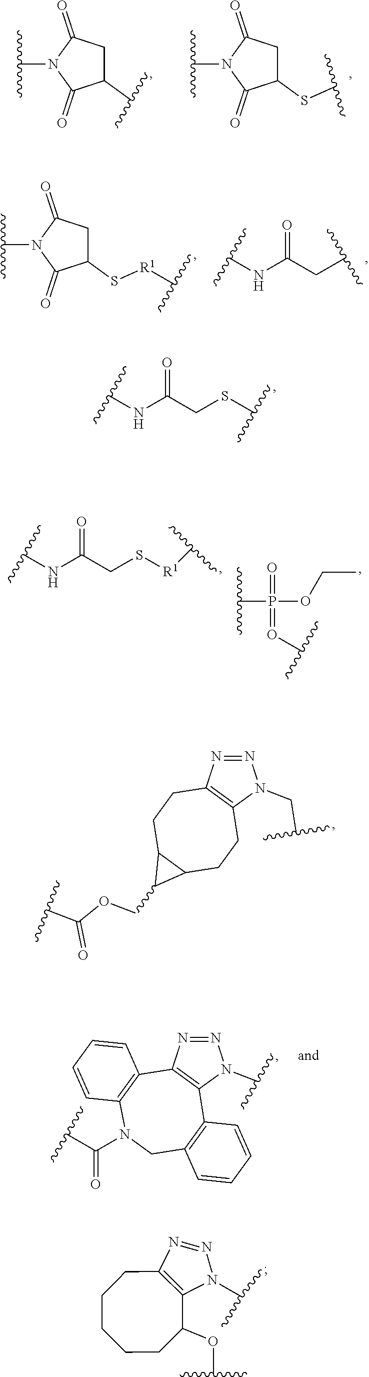
wherein: R¹ is alkylene, cycloalkyl, or 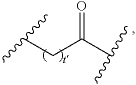
wherein t′ is 0 to 10 wherein each R is independently an alkyl, alkenyl, alkynyl, carbocyclyl, or heterocyclyl, wherein R¹ is 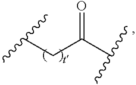
and t′ is 2.

48. The method of claim 46, wherein M is —COOH.

49. The method of claim 46, wherein the compound of formula (IV) is 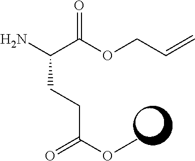
and the compound of formula (VI) is

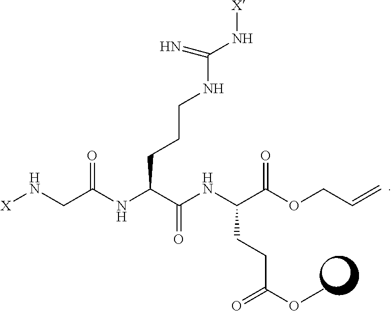

50. The method of claim 46, wherein the method of making the cCPP comprises:
(a) loading a Fmoc-Glu-OAll onto a 2-chlorotrityl chloride (CTC) resin to give a first Fmoc protected product: 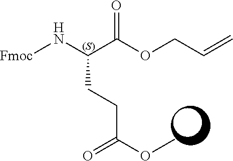
wherein 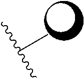
represents the resin;
(b) removing the Fmoc protecting group to obtain a first deprotected product; and
(c) coupling the first deprotected product with Fmoc-Gly-Arg(Pbf)-OH to give a second Fmoc protected product:

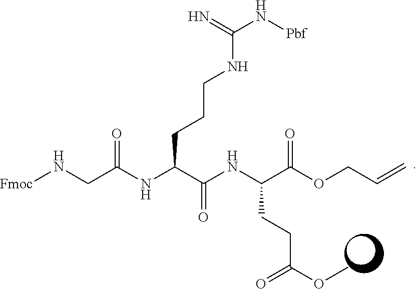

51. The method of claim 50, further comprising deprotecting the second Fmoc protected product: 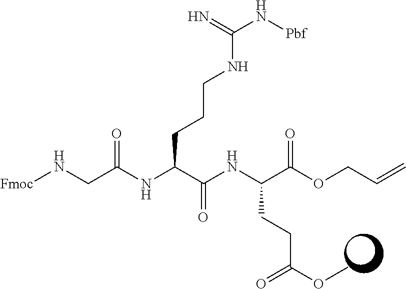
to obtain a second deprotected product and
coupling the second deprotected product with Fmoc protected amino acids (Fmoc-AA) sequentially to obtain:

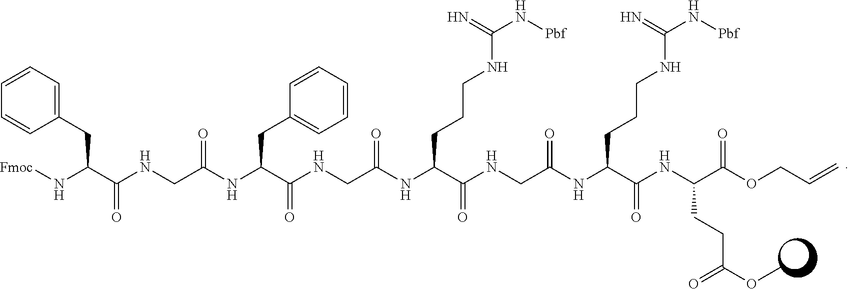

52. The method of claim 51, further comprising:
removing the allyl protecting group of the compound of formula: 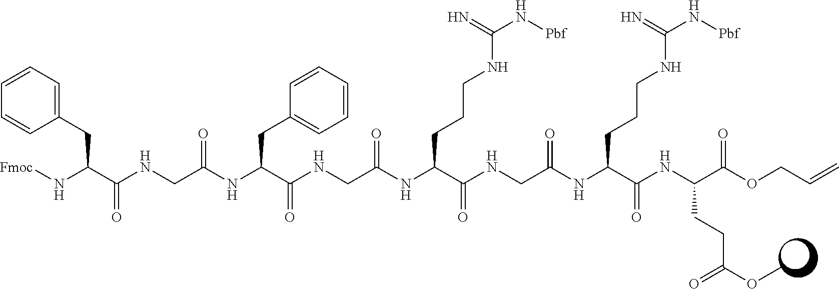
to give: 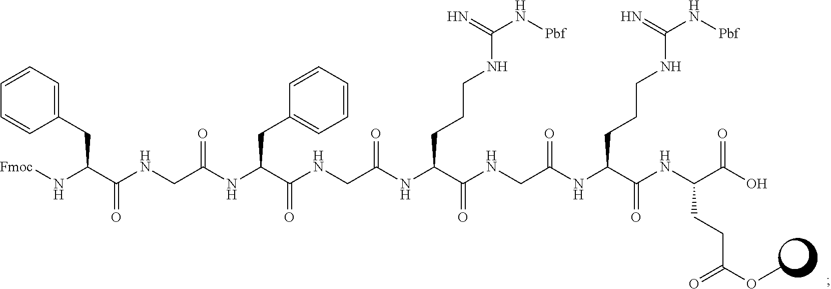
removing the Fmoc to give: 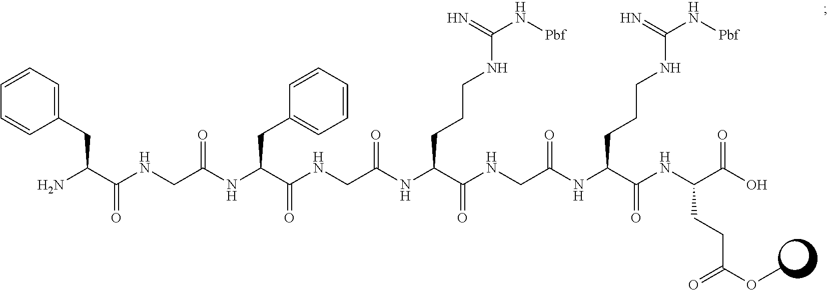
cyclizing the compound of the formula: 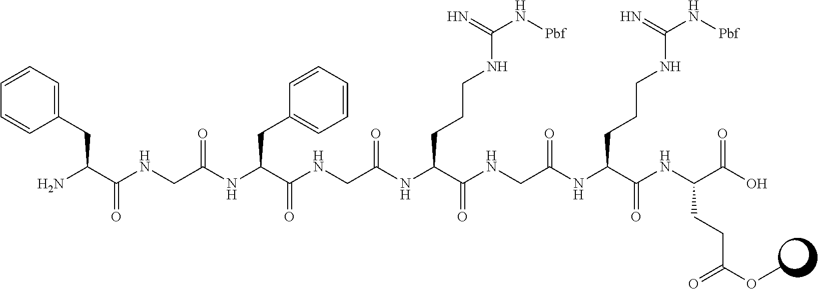
to give: 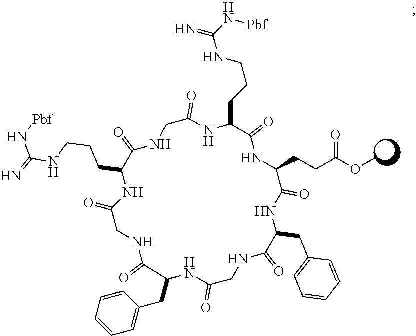
and
cleaving the resin from the compound of the compound of the formula: 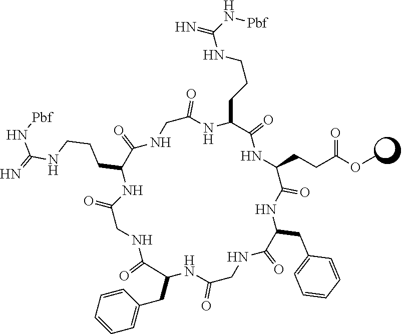
to give:

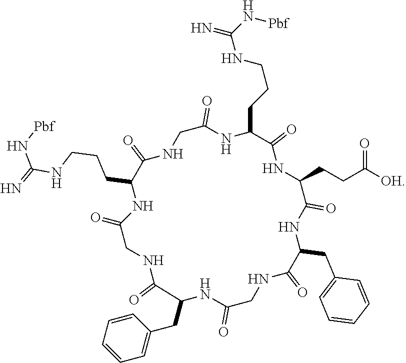

53. The method of claim 46, wherein (ii) making the linear peptide comprises deprotecting each sequentially coupled Fmoc protected amino acid.

54. The method of claim 46, wherein R₄ is H or a side chain of tyrosine, phenylalanine or tryptophan.

55. The method of claim 46, wherein two of R₁, R₂, R₃, and R₄ are phenylalanine side chains.

56. The method of claim 46, wherein two of R₁, R₂, R₃, and R₄ are H.

57. The method of claim 46, wherein the cCPP comprises Formula (I-1) or (I-2): 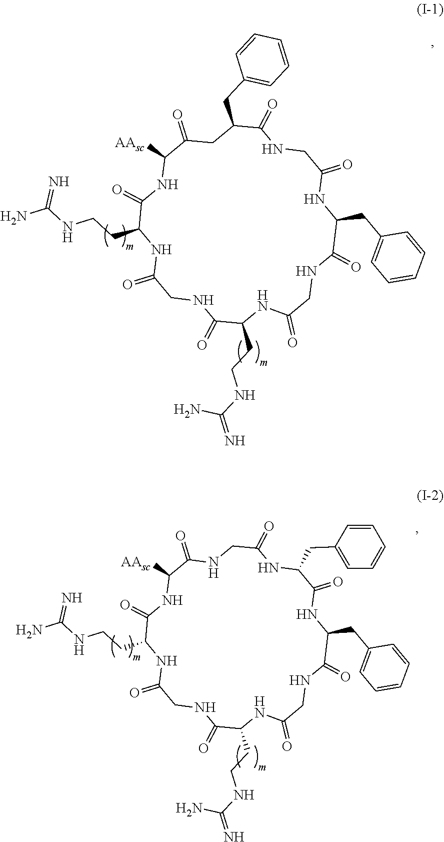
or a protonated form thereof.

58. The method of claim 46, wherein AA_{SC} is a side chain of an asparagine side chain, aspartate side chain, glutamine side chain, glutamate acid side chain, homoglutamine reside, or homoglutamate side chain.

59. The method of claim 46, wherein the EP comprises 1 or 2 arginine residues; and/or 2, 3, or 4 lysine residues.

60. The method of claim 46, wherein the amino group on the side chain of each lysine side chain of the EP is substituted with a trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (Dde) group.

61. The method of claim 46, wherein the exocyclic peptide comprises one of the following sequences:
PKKKRKV; KR; RR, KKK; KGK; KBK; KBR; KRK; KRR; RKK; RRR; KKKK; KKRK; KRKK; KRRK; RKKR; RRRR; KGKK; KKGK; KKKKK; KKKRK; KBKBK; KKKRKV; PGKKRKV; PKGKRKV; PKKGRKV; PKKKGKV; PKKKRGV; or PKKKRKG.

62. The method of claim 46, wherein the exocyclic peptide comprises PKKKRKV.

63. The method of claim 46, wherein the EEV is a compound of the formula (B): 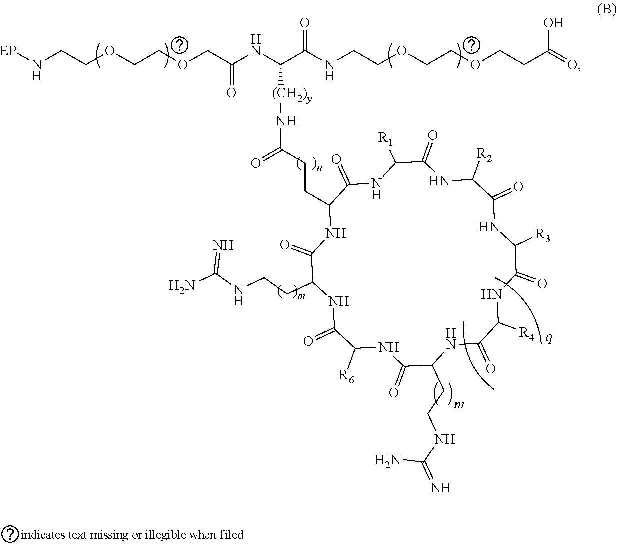
or protonated form thereof, wherein y is an integer from 1-5 and n is an integer from 0-2.

64. The method of claim 46, wherein the linear peptide comprises:

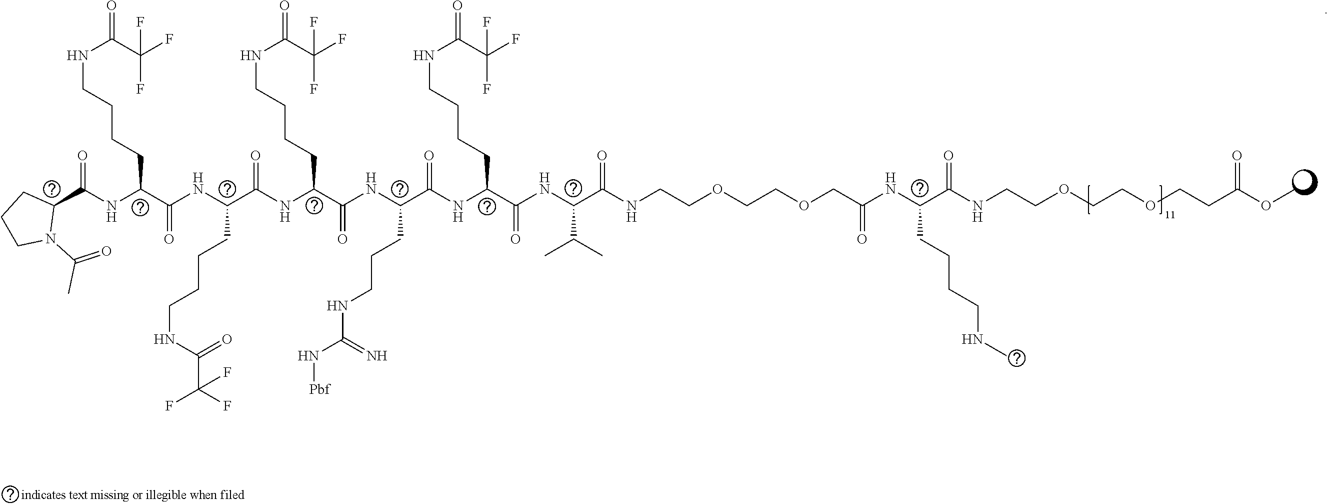

65. The method of claim 46, wherein (iii) coupling the cCPP to the linear peptide is performed in the presence of 3H-[1,2,3]Triazolo[4,5-b]pyridin-3-ol (HOAt) and N,N′-Diisopropylcarbodiimide (DIC); and an additional 3 equivalents of HOAt and an additional 3.3 equivalents of DIC.

66. The method of claim 46, wherein (iii) coupling the cCPP to the linear peptide is performed in the presence of 3 equivalents of 3H-[1,2,3]Triazolo[4,5-b]pyridin-3-ol (HOAt) and 3.3 equivalents of N,N′-Diisopropylcarbodiimide (DIC); and
an additional 3 equivalents of HOAt and an additional 3.3 equivalents of DIC.

67. The method of claim 46, wherein (iii) coupling the cCPP to the linear peptide comprises:
coupling a cyclic peptide of the formula: 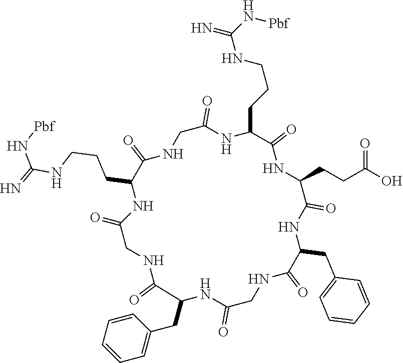
onto a linear peptide of the formula: 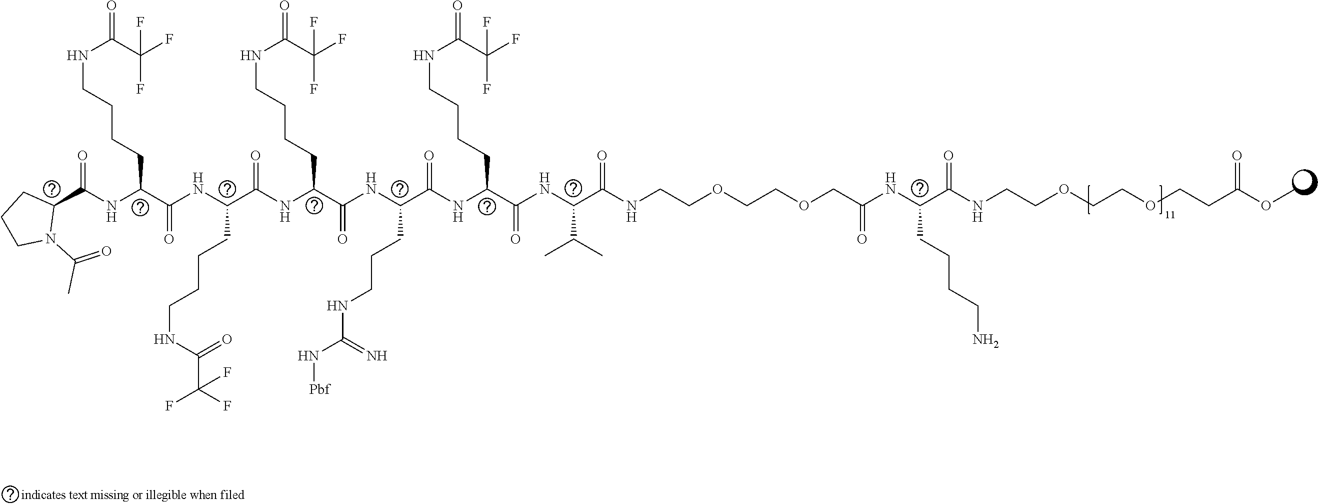
to give: 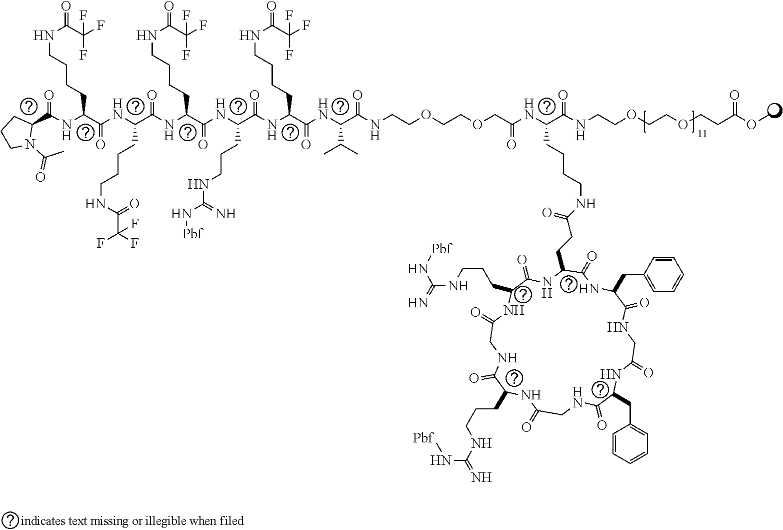
wherein:
the coupling is performed in the presence of 3 equivalents of 3H-[1,2,3]Triazolo[4,5-b]pyridin-3-ol (HOAt) and 3.3 equivalents of N,N′-Diisopropylcarbodiimide (DIC); and
an additional 3 equivalents of HOAt and an additional 3.3 equivalents of DIC.

68. The method of claim 67, further comprising cleaving the resin from the compound of the formula: 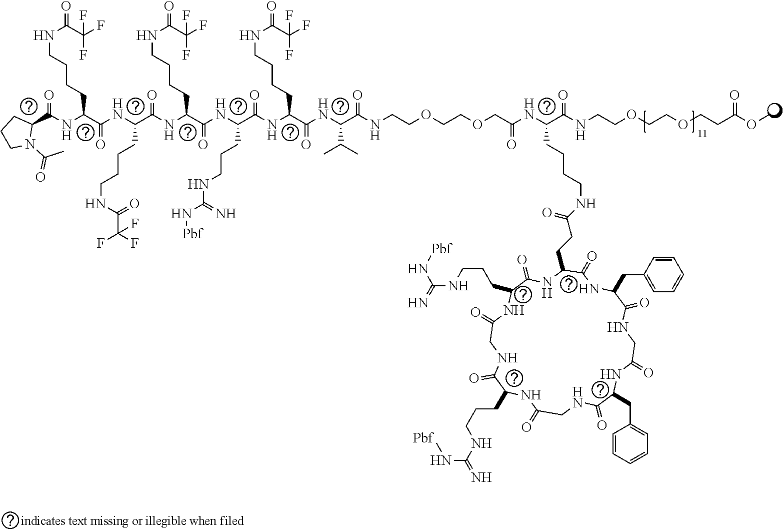
to give:

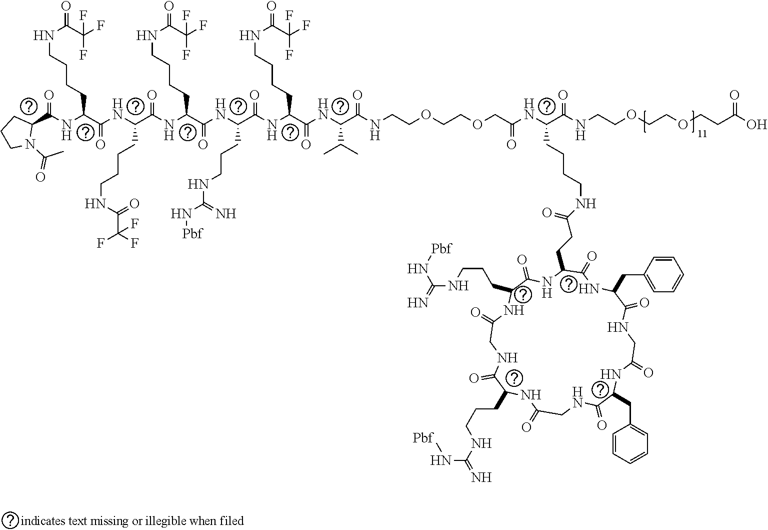

69. The method of claim 46, further comprising making a phosphorodiamidate morpholino oligomer (PMO) and conjugating the PMO to the EEV.

70. The method of claim 69, further comprising removing any protecting groups present on the conjugated product to give a deprotected conjugated product.

71. The method of claim 70, wherein the deprotected conjugated product is a compound of the Formula (C): 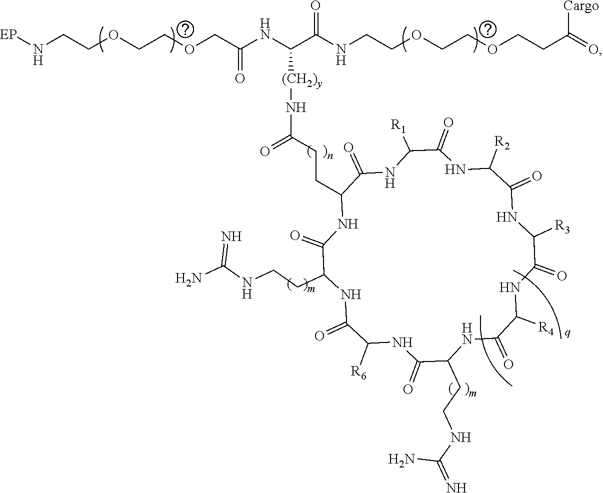
or a protonated form thereof, wherein y is an integer from 1-5, n is an integer from 0-2 and cargo is a PMO.

72. The method of claim 70, wherein the deprotected conjugated product is a compound of Formula (C-1): 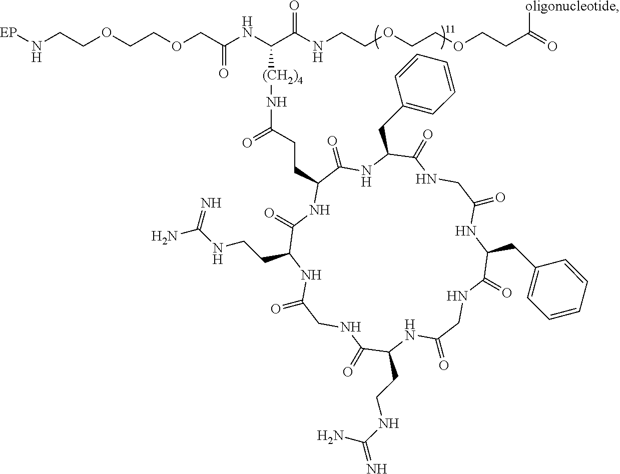
or a protonated form thereof, wherein oligonucleotide is a PMO.
